(19) United States
(12) Patent Application Publication
(10) Pub. No.: US 2025/0026841 A1
(43) Pub. Date: Jan. 23, 2025

(54) BISPECIFIC ANTIBODIES TARGETING CD137 AND USES THEREOF FOR ANTI-CANCER IMMUNOTHERAPY

(71) Applicant: AP Biosciences, Inc., Taipei City (TW)

(72) Inventors: Jeng-Horng Her, San Jose, CA (US); Po-Lin Huang, Taipei City (TW); Hsin-Ta Hsieh, Taipei City (TW); Ching-Hsuan Hsu, Taipei City (TW); Jhong-Jhe You, Taipei City (TW)

(21) Appl. No.: 18/708,551

(22) PCT Filed: Nov. 18, 2022
(86) PCT No.: PCT/US2022/050472

§ 371 Date: May 8, 2024

Related U.S. Application Data
(60) Provisional application No. 63/392,474, filed on Jul. 26, 2022.
(60) Provisional application No. 63/281,347, filed on Nov. 19, 2021.

(51) Int. Cl. C07K 16/28; A61K 47/68; C07K 16/32

(52) U.S. Cl. CPC C07K 16/2878; A61K 47/6801; C07K 16/2896; C07K 16/32; C07K 2317/31; C07K 2317/622; C07K 2317/732; C07K 2317/92

(57) ABSTRACT

Provided herein are antibodies or antigen binding fragments thereof having a binding specificity for p95HER2 or for CD73, bispecific antibodies comprising a first antigen binding region that binds to p95HER2 or CD73 and a second antigen binding region that binds to an immune checkpoint molecule or an immune stimulatory molecule, and antibody-drug conjugates thereof. Also provided herein are pharmaceutical compositions comprising the antibodies, antigen binding fragments thereof, bispecific antibodies or antibody drug conjugates thereof, and methods of use thereof. The methods of use include method of treating cancer.

### CROSS-REFERENCE TO RELATED APPLICATIONS

This application claims benefit of priority under 35 U.S.C. § 119 (c) of U.S. Provisional Applications Nos. 63/281,347, filed Nov. 19, 2021, and 63/392,474, filed Jul. 26, 2022. The disclosure of the prior applications is considered part of and are herein incorporated by reference in the disclosure of this application in their entirety.

### INCORPORATION OF SEQUENCE LISTING

The material in the accompanying sequence listing is hereby incorporated by reference into this application. The accompanying sequence listing XML file, name AP1110_2WO.xml, was created on Nov. 1, 2022, and is 206 kb.

### BACKGROUND OF THE INVENTION

### Field of the Invention

The present invention relates generally to antibodies and antigen binding fragments thereof and more specifically to antibodies and antigen binding fragments having cytotoxicity against cancer cells and enhancing T-cell function.

### Background Information

Cancer immunotherapy restores or boosts the natural defense against tumor by the human immune system. Such therapy usually targets specific biological molecules on cancer cells' surface such as tumor-associated antigens (TAAs). Anti-tumor activities are possibly achieved by directing the host immune system toward TAAs, which consequently establishes or induces adaptive immune responses against cancer cells. Over the past few decades, cancer treatment using monoclonal antibodies (mAbs) has achieved great success and many of them have been approved for cancer treatment or clinical trials.

CD137 (4-1BB/TNFRSF9) was identified in 1989 as an inducible gene that was expressed on antigen-primed T cells but not on resting ones. In addition, it is known to be expressed in dendritic (DCs), natural killer cells (NKs), activated CD4+ and CD8+T lymphocytes, cosinophils, natural killer T cells (NKTs), and mast cells, but myeloid-derived suppressor cells (MDSCs) were investigated to not express this molecule on their surfaces. The anti-CD137 antibodies show great potential in anti-cancer activities due to its ability to activate cytotoxic T cells and to increase the production of interferon gamma (IFN-γ).

Antibody-based strategies for cancer treatment have dramatically advanced in the past 20 years. Since rituximab was approved as the first monoclonal antibody (mAb) for the treatment of cancer in 1997, several mAbs have become standard of care for the treatment of both solid tumors and hematologic malignancies. Most of the approved mAbs (e.g., rituximab, trastuzumab, and cetuximab) target tumor-associated antigens on the surface of cancer cells and inhibit cell growth. Although several effective antibodies have emerged, long-term, durable responses remain elusive, and resistance and relapse remain major problems. Immunomodulatory antibodies have revolutionized cancer immunotherapy and helped garner the breakthrough distinction.

In addition to checkpoint blockade agents such as ipilimumab and pembrolizumab, agents targeting the tumor necrosis factor (TNF) superfamily of costimulatory receptors have entered development. CD137 is one of the TNF receptor family targets that have advanced into clinical trials. CD137 regulates many immune cells, including CD4+ and CD8+ T cells, regulatory T cells (Treg), dendritic cells (DC), and natural killer (NK) cells. Recent studies indicate that the addition of anti-CD137 mAbs can augment the antitumor efficacy of immunomodulatory antibodies. However, there is still an unmet need for more specific and more effective antigen binding molecules that target CD137.

### SUMMARY OF THE INVENTION

The present innovation is based on the discovery of anti-p95HER2 antibodies that show a superior activity to bind p95HER2-expressing cancer cells and of anti-CD73 antibodies that show a superior activity to neutralize the enzymatic activity of CD73 in the cell membrane-bound or soluble form. The present invention is further based on the discovery that bispecific antibodies targeting p95HER2 and CD137 possess dual functions, antibody antibody-dependent cell-mediated cytotoxicity (ADCC) and p95HER2-dependent T-cell activation, against p95HER-positive cancer cells, and that bispecific antibodies targeting CD73 and CD137 possess extraordinary activity to boost T-cell effector function and inhibit tumor growth in vivo better than mono-treatment or combination treatment with each antibody.

In one embodiment, the invention provides four antibodies or antigen binding fragments thereof: anti-p95HER2 antibody clone R4-3 (p95HER2 #R4-3), anti-p95HER2 antibody clone R4-5 (p95HER2 #R4-5), anti-p95HER2 antibody clone R4-11 (p95HER2 #R4-11), anti-p95HER2 antibody clone R4-15 (p95HER2 #R4-15), anti-p95HER2 antibody clone R4-38 (p95HER2 #R4-38). In one aspect, the anti-p95HER2 antibodies provided herein include a heavy chain variable (VH) region including an amino acid sequence with at least 80% sequence identity to a sequence selected from SEQ ID NO:1, SEQ ID NO:9, SEQ ID NO:17, SEQ ID NO:25 and SEQ ID NO:33; and a light chain variable (VL) region including an amino acid sequence with at least 80% sequence identity to a sequence selected from SEQ ID NO:2, SEQ ID NO: 10, SEQ ID NO:18, SEQ ID NO:26 and SEQ ID NO:34.

In one aspect, the antibody or antigen binding fragment has a VH region that includes an amino acid sequence having at least 80% identity to SEQ ID NO: 1 and a VL region that includes an amino acid sequence having at least 80% identity to SEQ ID NO:2 includes (a) VH CDR-H1, CDR-H2, and CDR-H3, wherein CDR-H1 includes an amino acid sequence having at least 80% identity to SEQ ID NO:3, wherein CDR-H2 includes an amino acid sequence having at least 80% identity to SEQ ID NO:4, and wherein CDR-H3 includes an amino acid sequence having at least 80% identity to SEQ ID NO:5; and (b) VL CDR-L1, CDR-L2, and CDR-L3, wherein CDR-L1 includes an amino acid sequence having at least 80% identity to SEQ ID NO:6, wherein CDR-L2 includes an amino acid sequence having at least 80% identity to SEQ ID NO:7, and wherein CDR-L3 includes an amino acid sequence having at least 80% identity to SEQ ID NO:8.

In another aspect, the antibody or antigen binding fragment thereof has a VH region that includes an amino acid sequence having at least 80% identity to SEQ ID NO:9 and a VL region that includes an amino acid sequence having at least 80% identity to SEQ ID NO:10 includes (a) VH CDR-H1, CDR-H2, and CDR-H3, wherein CDR-H1 includes an amino acid sequence having at least 80% identity to SEQ ID NO:11, wherein CDR-H2 includes an amino acid sequence having at least 80% identity to SEQ ID NO:12, and wherein CDR-H3 includes an amino acid sequence having at least 80% identity to SEQ ID NO:13; and (b) VL CDR-L1, CDR-L2, and CDR-L3, wherein CDR-L1 includes an amino acid sequence having at least 80% identity to SEQ ID NO:14, wherein CDR-L2 includes an amino acid sequence having at least 80% identity to SEQ ID NO:15, and wherein CDR-L3 includes an amino acid sequence having at least 80% identity to SEQ ID NO:16.

In one aspect, the antibody or antigen binding fragment thereof has a VH region that includes an amino acid sequence having at least 80% identity to SEQ ID NO:17 and a VL region that includes an amino acid sequence having at least 80% identity to SEQ ID NO:18 includes (a) VH CDR-H1, CDR-H2, and CDR-H3, wherein CDR-H1 includes an amino acid sequence having at least 80% identity to SEQ ID NO:19, wherein CDR-H2 includes an amino acid sequence having at least 80% identity to SEQ ID NO:20, and wherein CDR-H3 includes an amino acid sequence having at least 80% identity to SEQ ID NO:21; and (b) VL CDR-L1, CDR-L2, and CDR-L3, wherein CDR-L1 includes an amino acid sequence having at least 80% identity to SEQ ID NO:22, wherein CDR-L2 includes an amino acid sequence having at least 80% identity to SEQ ID NO:23, and wherein CDR-L3 includes an amino acid sequence having at least 80% identity to SEQ ID NO:24.

In another aspect, the antibody or antigen binding fragment thereof has a VH region that includes an amino acid sequence having at least 80% identity to SEQ ID NO:25 and a VL region that includes an amino acid sequence having at least 80% identity to SEQ ID NO:26 includes (a) VH CDR-H1, CDR-H2, and CDR-H3, wherein CDR-H1 includes an amino acid sequence having at least 80% identity to SEQ ID NO:27, wherein CDR-H2 includes an amino acid sequence having at least 80% identity to SEQ ID NO:28, and wherein CDR-H3 includes an amino acid sequence having at least 80% identity to SEQ ID NO:29; and (b) VL CDR-L1, CDR-L2, and CDR-L3, wherein CDR-L1 includes an amino acid sequence having at least 80% identity to SEQ ID NO:30, wherein CDR-L2 includes an amino acid sequence having at least 80% identity to SEQ ID NO:31, and wherein CDR-L3 includes an amino acid sequence having at least 80% identity to SEQ ID NO:32.

In yet another aspect, the antibody or antigen binding fragment thereof has a VH region that includes an amino acid sequence having at least 80% identity to SEQ ID NO:33 and a VL region that includes an amino acid sequence having at least 80% identity to SEQ ID NO: 34 includes (a) VH CDR-H1, CDR-H2, and CDR-H3, wherein CDR-H1 includes an amino acid sequence having at least 80% identity to SEQ ID NO:35, wherein CDR-H2 includes an amino acid sequence having at least 80% identity to SEQ ID NO:36, and wherein CDR-H3 includes an amino acid sequence having at least 80% identity to SEQ ID NO:37; and (b) VL CDR-L1, CDR-L2, and CDR-L3, wherein CDR-L1 includes an amino acid sequence having at least 80% identity to SEQ ID NO:38, wherein CDR-L2 includes an amino acid sequence having at least 80% identity to SEQ ID NO:39, and wherein CDR-L3 includes an amino acid sequence having at least 80% identity to SEQ ID NO:40.

In one aspect, the antibody includes an Fc domain. In some aspects, the Fc domain is an IgG, IgE, IgM, IgD, IgA, or IgY domain. In various aspects, the IgG domain is an IgG1, IgG2, IgG3, or IgG4 domain. In other aspects, the IgG1 domain includes an amino acid sequence of SEQ ID NO:41. In various aspects, the IgG1 domain includes point mutations compared to a wild-type IgG1 domain that modify or enhance antibody-dependent cellular cytotoxicity (ADCC) and/or complement-dependent cytotoxicity (CDC). In many aspects, point mutations include S241D/A332L/1334E mutations.

In another embodiment, the invention provides a bispecific antibody including a first antigen binding region and a second antigen binding region, wherein the first antigen binding region binds to p95HER2.

In one aspect, the bispecific antibody includes a V_{H} region including complementarity-determining region (CDR)-H1, CDR-H2, and CDR-H3, wherein CDR-H1 is set forth in SEQ ID NO:19, CDR-H2 is set forth in SEQ ID NO:20, and CDR-H3 is set forth in SEQ ID NO:21 or sequences having 90% identity to SEQ ID NO: 19, 20 or 21 and the antigen binding specificity thereof; and a V_{L} region including CDR-L1, CDR-L2, and CDR-L3, wherein CDR-L1 is set forth in SEQ ID NO:22, CDR-L2 is set forth in SEQ ID NO:23, and CDR-L3 is set forth in SEQ ID NO:24 or sequences having 90% identity to SEQ ID NO: 19, 20 or 21 and the antigen binding specificity thereof. In another aspect, the VH region has an amino acid sequence having at least 80% identity to SEQ ID NO:17 and an antigen binding specificity of SEQ ID NOs:19-21 and the VL region has an amino acid sequence having at least 80% identity to SEQ ID NO:18 and an antigen binding specificity of SEQ ID NOs: 22-24. In one aspect, the second antigen binding region binds to an immune checkpoint molecule, or an immune stimulatory molecule. In another aspect, the antibody includes a heavy chain sequence having the amino acid sequence of SEQ ID NO:46 or 47 or a sequence having 90% identity thereto and the binding specificity thereof. In one aspect, the bispecific antibody further includes a light chain having the amino acid sequence of SEQ ID NO:18. In another aspect, the bispecific antibody includes an Fc domain. In some aspects, the Fc domain is an IgG domain, an IgE domain, an IgM domain, and IgD domain, an IgA domain, or an IgY domain. In various aspects, the Fc domain is an IgG domain. In many aspects, the IgG domain is an IgG1 domain, an IgG2 domain, an IgG3 domain, or an IgG4 domain. In one aspect, a scFv is linked to the C-terminus of the Fc domain. In another aspect, the bispecific antibody includes a linker between the Fab domain and the scFv domain. In some aspects, the Fab fragment is linked to the N-terminus of the Fc domain. In various aspects, the Fab includes a p95HER2 binding site, and the scFv includes a CD137 binding site.

In one embodiment, the invention provides an antibody-drug conjugate including: a therapeutic agent, and any one of the antibodies or antigen binding fragments thereof described herein, or any one of the bispecific antibodies described herein.

In one aspect, the therapeutic agent is covalently linked to the antibody, antigen binding fragment or bispecific antibody via a linker.

In another embodiment, the invention provides a pharmaceutical composition including any one of the antibodies or antigen binding fragments thereof described herein, any one of the bispecific antibodies described herein or any one of the antibody-drug conjugates described herein, and at least one pharmaceutical acceptable carrier.

In one aspect, the pharmaceutically acceptable carrier is conjugated to the C-terminus of the C-terminus of the amino acid sequence of the antibody, antigen binding fragment, bispecific antibody, or antibody-drug conjugate.

In one embodiment, the invention provides a method of treating cancer including administering to a subject in need thereof an effective amount of any one of the antibodies or antigen binding fragments thereof described herein, any one of the bispecific antibodies described herein, or any one of the antibody-drug conjugates described herein, thereby treating cancer. In one aspect, the cancer is selected from prostate cancer, lung cancer, Non-Small Cell Lung Cancer (NSCLC), melanoma, lymphoma, breast cancer, head and neck cancer, renal cell carcinoma (RCC), ovarian cancer, kidney cancer, urinary bladder cancer, uterine cancer, cervical cancer, ovarian cancer, liver cancer, stomach cancer, colon cancer, rectal cancer, oral cavity cancer, pharynx cancer, pancreatic cancer, thyroid cancer, skin cancer, brain cancer, bone cancer, hematopoietic cancer, or leukemia.

In another embodiment, the invention provides an isolated amino acid sequence as set forth in SEQ ID NOs: 1-42 or 46-47.

In one embodiment, the invention provides an isolated nucleic acid sequence encoding any one of the antibodies or antigen-binding fragments thereof, or the bispecific antibodies described herein. In another embodiment, the invention provides an isolated nucleic acid encoding any one of SEQ ID NOs: 1-42 or 46-47.

In a further embodiment, the invention provides four antibodies or antigen binding fragments thereof: anti-CD73 antibody clone 3-4 (CD73 #3-4), anti-CD73 antibody clone 3-16 (CD73 #3-16), anti-CD73 antibody clone 3-1-28 (CD73 #3-1-28), and anti-CD73 antibody clone 3-1-37 (CD73 #3-1-37); and three affinity-maturated antibodies derived from anti-CD73 clone 3-1-28: anti-CD73 #3-1-28 (S51A Q54E), anti-CD73 #3-1-28 (S107D S109H), anti-CD73 #3-1-28 (S107D S109T A114S).

In one aspect, the anti-CD73 antibodies provided herein include a heavy chain variable (VH) region including an amino acid sequence with at least 80% sequence identity to a sequence selected from SEQ ID NO:49, SEQ ID NO:57, SEQ ID NO:65, and SEQ ID NO:73; and a light chain variable (VL) region including an amino acid sequence with at least 80% sequence identity to a sequence selected from SEQ ID NO:50, SEQ ID NO:58, SEQ ID NO:66 and SEQ ID NO:74, SEQ ID NO:81, SEQ ID NO:83 and SEQ ID NO:85.

In one aspect, the antibody or antigen binding fragment has a VH region that includes an amino acid sequence having at least 80% identity to SEQ ID NO:49 and a VL region that includes an amino acid sequence having at least 80% identity to SEQ ID NO:50 includes (a) VH CDR-H1, CDR-H2, and CDR-H3, wherein CDR-H1 includes an amino acid sequence having at least 80% identity to SEQ ID NO:51, wherein CDR-H2 includes an amino acid sequence having at least 80% identity to SEQ ID NO:52, and wherein CDR-H3 includes an amino acid sequence having at least 80% identity to SEQ ID NO:53; and (b) VL CDR-L1, CDR-L2, and CDR-L3, wherein CDR-L1 includes an amino acid sequence having at least 80% identity to SEQ ID NO:54, wherein CDR-L2 includes an amino acid sequence having at least 80% identity to SEQ ID NO:55, and wherein CDR-L3 includes an amino acid sequence having at least 80% identity to SEQ ID NO:56.

In another aspect, the antibody or antigen binding fragment thereof has a VH region that includes an amino acid sequence having at least 80% identity to SEQ ID NO:57 and a VL region that includes an amino acid sequence having at least 80% identity to SEQ ID NO:58 includes (a) VH CDR-H1, CDR-H2, and CDR-H3, wherein CDR-H1 includes an amino acid sequence having at least 80% identity to SEQ ID NO:59, wherein CDR-H2 includes an amino acid sequence having at least 80% identity to SEQ ID NO:60, and wherein CDR-H3 includes an amino acid sequence having at least 80% identity to SEQ ID NO:61; and (b) VL CDR-L1, CDR-L2, and CDR-L3, wherein CDR-L1 includes an amino acid sequence having at least 80% identity to SEQ ID NO:62, wherein CDR-L2 includes an amino acid sequence having at least 80% identity to SEQ ID NO:63, and wherein CDR-L3 includes an amino acid sequence having at least 80% identity to SEQ ID NO:64.

In one aspect, the antibody or antigen binding fragment thereof has a VH region that includes an amino acid sequence having at least 80% identity to SEQ ID NO:65 and a VL region that includes an amino acid sequence having at least 80% identity to SEQ ID NO:66 includes (a) VH CDR-H1, CDR-H2, and CDR-H3, wherein CDR-H1 includes an amino acid sequence having at least 80% identity to SEQ ID NO:67, wherein CDR-H2 includes an amino acid sequence having at least 80% identity to SEQ ID NO:68, and wherein CDR-H3 includes an amino acid sequence having at least 80% identity to SEQ ID NO:69; and (b) VL CDR-L1, CDR-L2, and CDR-L3, wherein CDR-L1 includes an amino acid sequence having at least 80% identity to SEQ ID NO:70, wherein CDR-L2 includes an amino acid sequence having at least 80% identity to SEQ ID NO:71, and wherein CDR-L3 includes an amino acid sequence having at least 80% identity to SEQ ID NO:72.

In another aspect, the antibody or antigen binding fragment thereof has a VH region that includes an amino acid sequence having at least 80% identity to SEQ ID NO:73 and a VL region that includes an amino acid sequence having at least 80% identity to SEQ ID NO: 74 includes (a) VH CDR-H1, CDR-H2, and CDR-H3, wherein CDR-H1 includes an amino acid sequence having at least 80% identity to SEQ ID NO:75, wherein CDR-H2 includes an amino acid sequence having at least 80% identity to SEQ ID NO:76, and wherein CDR-H3 includes an amino acid sequence having at least 80% identity to SEQ ID NO:77; and (b) VL CDR-L1, CDR-L2, and CDR-L3, wherein CDR-L1 includes an amino acid sequence having at least 80% identity to SEQ ID NO:78, wherein CDR-L2 includes an amino acid sequence having at least 80% identity to SEQ ID NO:79, and wherein CDR-L3 includes an amino acid sequence having at least 80% identity to SEQ ID NO:80.

In an additional embodiment, the present invention provides three affinity-maturated antibodies derived from anti-CD73 clone 3-1-28: anti-CD73 #3-1-28 (S51A Q54E), anti-CD73 #3-1-28 (S107D S109H), anti-CD73 #3-1-28 (S107D S109T A114S).

In one aspect, the anti-CD73 #3-1-28 (S51A Q54E) antibody or antigen binding fragment thereof having a VL region that includes an amino acid sequence having at least 80% identity to SEQ ID NO:81 includes VL CDR-L2 region that includes an amino acid sequence having at least 80% identity to SEQ ID NO:82. In another aspect, the anti-CD73 #3-1-28 (S107D S109H) antibody or antigen binding fragment thereof having a VH region that includes an amino acid sequence having at least 80% identity to SEQ ID NO:83 includes VH CDR-H3 region that includes an amino acid sequence having at least 80% identity to SEQ ID NO:84. In yet another aspect, the anti-CD73 #3-1-28 (S107D S109T A114S) antibody or antigen binding fragment thereof having a VH region that includes an amino acid sequence having at least 80% identity to SEQ ID NO:85 includes VH CDR-H3 region that includes an amino acid sequence having at least 80% identity to SEQ ID NO:86.

In one aspect, the antibody or antigen binding fragment thereof has a VH region that includes an amino acid sequence having at least 80% identity to SEQ ID NO:65 and a VL region that includes an amino acid sequence having at least 80% identity to SEQ ID NO:81 includes (a) VH CDR-H1, CDR-H2, and CDR-H3, wherein CDR-H1 includes an amino acid sequence having at least 80% identity to SEQ ID NO:67, wherein CDR-H2 includes an amino acid sequence having at least 80% identity to SEQ ID NO:68, and wherein CDR-H3 includes an amino acid sequence having at least 80% identity to SEQ ID NO:69; and (b) VL CDR-L1, CDR-L2, and CDR-L3, wherein CDR-L1 includes an amino acid sequence having at least 80% identity to SEQ ID NO:70, wherein CDR-L2 includes an amino acid sequence having at least 80% identity to SEQ ID NO:82, and wherein CDR-L3 includes an amino acid sequence having at least 80% identity to SEQ ID NO:72.

In another aspect, the antibody or antigen binding fragment thereof has a VH region that includes an amino acid sequence having at least 80% identity to SEQ ID NO:65 and a VL region that includes an amino acid sequence having at least 80% identity to SEQ ID NO:83 includes (a) VH CDR-H1, CDR-H2, and CDR-H3, wherein CDR-H1 includes an amino acid sequence having at least 80% identity to SEQ ID NO:67, wherein CDR-H2 includes an amino acid sequence having at least 80% identity to SEQ ID NO:68, and wherein CDR-H3 includes an amino acid sequence having at least 80% identity to SEQ ID NO:69; and (b) VL CDR-L1, CDR-L2, and CDR-L3, wherein CDR-L1 includes an amino acid sequence having at least 80% identity to SEQ ID NO:70, wherein CDR-L2 includes an amino acid sequence having at least 80% identity to SEQ ID NO:84, and wherein CDR-L3 includes an amino acid sequence having at least 80% identity to SEQ ID NO:72.

In yet another aspect, the antibody or antigen binding fragment thereof has a VH region that includes an amino acid sequence having at least 80% identity to SEQ ID NO:65 and a VL region that includes an amino acid sequence having at least 80% identity to SEQ ID NO: 85 includes (a) VH CDR-H1, CDR-H2, and CDR-H3, wherein CDR-H1 includes an amino acid sequence having at least 80% identity to SEQ ID NO:67, wherein CDR-H2 includes an amino acid sequence having at least 80% identity to SEQ ID NO:68, and wherein CDR-H3 includes an amino acid sequence having at least 80% identity to SEQ ID NO:69; and (b) VL CDR-L1, CDR-L2, and CDR-L3, wherein CDR-L1 includes an amino acid sequence having at least 80% identity to SEQ ID NO:70, wherein CDR-L2 includes an amino acid sequence having at least 80% identity to SEQ ID NO:86, and wherein CDR-L3 includes an amino acid sequence having at least 80% identity to SEQ ID NO:72.

In one aspect, antibodies or antigen binding fragments thereof provided herein include an Fc domain. In some aspects, the Fc domain is an IgG, IgE, IgM, IgD, IgA, or IgY domain. In various aspects, the IgG domain is an IgG1, IgG2, IgG3, or IgG4 domain. In other aspects, the IgG1 domain includes an amino acid sequence of SEQ ID NO:87. In various aspects, IgG1 includes point mutations compared to wild-type IgG1 that modify or reduce antibody-dependent cellular cytotoxicity (ADCC) and/or complement-dependent cytotoxicity (CDC). In many aspects, point mutations include K297A and K322A mutations.

In another embodiment, the invention provides a bispecific antibody including a first antigen binding region and a second antigen binding region, wherein the first antigen binding region binds to CD73.

In one aspect, the bispecific antibody includes a VH region including complementarity-determining region (CDR)-H1, CDR-H2, and CDR-H3, wherein CDR-H1 is set forth in SEQ ID NO:67, CDR-H2 is set forth in SEQ ID NO:68, and CDR-H3 is set forth in SEQ ID NO:69 or sequences having 90% identity to SEQ ID NO:67, 68 or 69 and the antigen binding specificity thereof; and a VL region including CDR-L1, CDR-L2, and CDR-L3, wherein CDR-L1 is set forth in SEQ ID NO:70, CDR-L2 is set forth in SEQ ID NO:71, and CDR-L3 is set forth in SEQ ID NO:72 or sequences having 90% identity to SEQ ID NO:70, 71 or 72 and the antigen binding specificity thereof. In another aspect, the bispecific antibody includes a VH region including complementarity-determining region (CDR)-H1, CDR-H2, and CDR-H3, wherein CDR-H1 is set forth in SEQ ID NO:67, CDR-H2 is set forth in SEQ ID NO: 68, and CDR-H3 is set forth in SEQ ID NO:69 or sequences having 90% identity to SEQ ID NO: 67, 68 or 69 and the antigen binding specificity thereof; and a VL region including CDR-L1, CDR-L2, and CDR-L3, wherein CDR-L1 is set forth in SEQ ID NO:70, CDR-L2 is set forth in SEQ ID NO:82, and CDR-L3 is set forth in SEQ ID NO:72 or sequences having 90% identity to SEQ ID NO:70, 82 or 72 and the antigen binding specificity thereof. In one aspect, the bispecific antibody includes a VH region including complementarity-determining region (CDR)-H1, CDR-H2, and CDR-H3, wherein CDR-H1 is set forth in SEQ ID NO:67, CDR-H2 is set forth in SEQ ID NO:68, and CDR-H3 is set forth in SEQ ID NO:69 or sequences having 90% identity to SEQ ID NO:67, 68 or 69 and the antigen binding specificity thereof; and a VL region including CDR-L1, CDR-L2, and CDR-L3, wherein CDR-L1 is set forth in SEQ ID NO:70, CDR-L2 is set forth in SEQ ID NO:84, and CDR-L3 is set forth in SEQ ID NO: 72 or sequences having 90% identity to SEQ ID NO:70, 84 or 72 and the antigen binding specificity thereof. In another aspect, the bispecific antibody includes a VH region including complementarity-determining region (CDR)-H1, CDR-H2, and CDR-H3, wherein CDR-H1 is set forth in SEQ ID NO:67, CDR-H2 is set forth in SEQ ID NO:68, and CDR-H3 is set forth in SEQ ID NO:69 or sequences having 90% identity to SEQ ID NO:67, 68 or 69 and the antigen binding specificity thereof; and a VL region including CDR-L1, CDR-L2, and CDR-L3, wherein CDR-L1 is set forth in SEQ ID NO:70, CDR-L2 is set forth in SEQ ID NO:86, and CDR-L3 is set forth in SEQ ID NO:72 or sequences having 90% identity to SEQ ID NO:70, 86 or 72 and the antigen binding specificity thereof. In one aspect, the VH region has an amino acid sequence having at least 80% identity to SEQ ID NO:65 and an antigen binding specificity of SEQ ID NOs: 67-69 and the VL region has an amino acid sequence having at least 80% identity to SEQ ID NO:66 and an antigen binding specificity of SEQ ID NOs: 70-72. In another aspect, the VH region has an amino acid sequence having at least 80% identity to SEQ ID NO: 65 and an antigen binding specificity of SEQ ID NOs: 67-69 and the VL region has an amino acid sequence having at least 80% identity to SEQ ID NO:81 and an antigen binding specificity of SEQ ID NOs: 70, 82 and 72. In one aspect, the VH region has an amino acid sequence having at least 80% identity to SEQ ID NO:65 and an antigen binding specificity of SEQ ID NOs: 67-69 and the VL region has an amino acid sequence having at least 80% identity to SEQ ID NO:82 and an antigen binding specificity of SEQ ID NOs: 70, 84 and 72. In another aspect, the VH region has an amino acid sequence having at least 80% identity to SEQ ID NO: 65 and an antigen binding specificity of SEQ ID NOs: 67-69 and the VL region has an amino acid sequence having at least 80% identity to SEQ ID NO:85 and an antigen binding specificity of SEQ ID NOs: 70, 86 and 72. In one aspect the second antigen binding region binds to an immune checkpoint molecule, or an immune stimulatory molecule. In one aspect, the antibody includes a heavy chain sequence having the amino acid sequence of SEQ ID NO: 88, 89, 90 or 91 or a sequence having 90% identity thereto and the binding specificity thereof. In another aspect, the bispecific antibody further includes a light chain having the amino acid sequence of SEQ ID NO:66.

In one embodiment, the invention provides an antibody-drug conjugate including: a therapeutic agent, and any one of the antibodies or antigen binding fragments thereof described herein, or any one of the bispecific antibodies described herein.

In another embodiment, the invention provides a pharmaceutical composition including any one of the antibodies or antigen binding fragments thereof described herein, any one of the bispecific antibodies described herein, or anyone of the antibody-drug conjugates described herein, and at least one pharmaceutically acceptable carrier.

In one embodiment, the invention provides a method of treating cancer including administering to a subject in need thereof an effective amount of any one of the antibodies or antigen binding fragments thereof described herein, any one of the bispecific antibodies described herein, or any one of the antibody-drug conjugates described herein, thereby treating cancer.

In another embodiment, the invention provides an isolated amino acid sequence as set forth in SEQ ID NOs: 49-86 or 88-91.

In one embodiment, the invention provides an isolated nucleic acid sequence encoding any one of the antibodies or antigen-binding fragments thereof, or the bispecific antibodies described herein. In another embodiment, the invention provides an isolated nucleic acid encoding any one of SEQ ID NOs: 49-86 or 88-91.

### BRIEF DESCRIPTION OF THE DRAWINGS

FIGS. 1A-1C show the screening for phage clones targeted to p95HER2 by flow cytometer. The phage clones that recognize p95HER2 were enriched after four rounds of panning. Positive phage binders were selected by staining p95HER2-HEK293 cells with phage-containing supernatant. FIG. 1A illustrates phage clones with specific binding to p95HER2-HEK293 cells were determined by flow cytometer. FIG. 1B illustrates clones (as numbered 1 to 40) specifically bound to p95HER2-HEK293 cells with ΔMFI higher than 800 were selected for sequencing. Negative control (NC), stained with secondary antibody alone. Positive control (PC), stained with both primary anti-p95HER2 ref antibody and secondary antibody. FIG. 1C illustrates phage clones with distinct sequences (marked in bold with grey background) were selected for conversion to monoclonal antibodies. Clones were named as R4-X, where X is the number in FIG. 1A.

FIG. 2 shows the purity of Protein A-purified anti-p95HER2 antibody leads by SDS-PAGE. The purity and integrity of one-step Protein A-purified anti-p95HER2 antibody leads was analyzed by SDS-PAGE under non-reducing and reducing conditions.

FIGS. 3A-3B show the binding activity of purified anti-p95HER2 antibody leads by flow cytometer. FIG. 3A is a graph illustrating that P95HER2-expressing HEK293 cells were surface stained with anti-p95HER2 antibody (30, 3, 0.3 nM) on ice for 1 hr then incubated with A488-conjugated anti-human IgG secondary antibody on ice for additional 1 hr. Cells were analyzed by flow cytometer, and A488 fluorescence intensity was shown by number. FIG. 3B is a graph illustrating that P95HER2-expressing NCI-H292 stable cells were surface stained with selected anti-p95HER2 antibodies and the analyzed by flow cytometer. Binding curve and EC50 were shown.

FIGS. 4A-4B show the binding epitope mapping of anti-p95HER2 antibody leads. FIG. 4A illustrates that anti-p95HER2 #R4-3, R4-5, R4-11, R4-15 and R4-38 antibodies showed widely identical responses against peptides with the consensus motif KFPDE at moderate to high spot intensities and high signal-to-noise ratios. Even though the intensity levels did not vary remarkably, antibody R4-3 showed a slightly weaker and antibody R4-38 the strongest response of these antibodies. FIG. 4B is a heat map showing anti-p95HER2 antibodies binding to linear 5, 10 and 15 amino acid peptides with peptide-peptide overlaps. Binding intensity was shown for each peptide, corrected with its own background value.

FIG. 5 shows the symmetric format of an anti-p95HER2-CD137 bispecific antibody (bsAb).

FIGS. 6A-6B show the protein aggregation and integrity of anti-p95HER2-CD137 bsAb by SEC-HPLC and μCE-SDS-PAGE, respectively. FIG. 6A shows the analysis of protein aggregation by SEC-HPLC. FIG. 6B shows the analysis of protein integrity by μCE-SDS-PAGE under non-reducing and reducing conditions, and the data were summarized in the table below.

FIGS. 7A-7B show the binding affinity of anti-p95HER2-CD137 bsAb against p95HER2 and CD137 antigen by biolayer interferometry. FIG. 7A illustrates binding kinetics against human p95HER2 antigen. FIG. 7B illustrates binding kinetics against human CD137 antigen.

FIG. 8 shows the simultaneous binding to p95HER2 and CD137 antigens by anti-p95HER2-CD137 bsAb.

FIGS. 9A-9B show that anti-p95HER2-CD137 bsAb induces IFN-γ production of T-cells upon coculturing with p95HER2-expressing NCI-H292 cells. FIG. 9A illustrates the expression level of p95HER2 on p95HER2-expressing NCI-H292 cells. FIG. 9B illustrates IFN-γ production of T cells upon coculturing with p95HER2-expressing NCI-H292 lung cancer cells.

FIGS. 10A-10B show the antibody-dependent cell-mediated cytotoxicity of anti-p95HER2-CD137 bsAb p95HER2-expressing NCI-H292 cells. FIG. 10A illustrates the expression level of CD137 on CD137-expressing HEK293 cells (left). ADCC of anti-p95HER2-CD137 bsAbs with normal or ADCC-enhancing IgG1 format (right). FIG. 10B illustrates the expression level of p95HER2 on p95HER2-expressing NCI-H292 cells (left). Anti-p95HER2-CD137 bsAb with normal IgG1 format induced a dose-dependent ADCC as well as #R4-11 IgG1 monoclonal antibody (right).

FIGS. 11A-11C show the anti-tumor activity of anti-p95HER2-CD137 bsAb by MDA-MB-231 breast cancer model in vivo. FIG. 11A shows p95HER2 expression by MDA=MB-231 cells. FIG. 11B illustrates tumor volume. FIG. 11C illustrates tumor mass.

FIG. 12 shows the pharmacokinetic profiles of anti-p95HER2-CD137 bsAb in vivo.

FIGS. 13A-13C shows the screening for phage clones targeted to CD73 by ELISA. FIG. 13A shows ELISA results of phage clones isolated from third-round MDA-MB-231 cell-based panning. FIG. 13B shows ELISA results of phage clones isolated from third-round CD73-overexpressing Expi293 cell-based panning in the third round. FIG. 13C shows a list of clone name numbered in FIGS. 13A and 13B.

FIG. 14 shows the purity of Protein A-purified anti-CD73 antibody leads by SDS-PAGE.

FIG. 15 shows the binding of anti-CD73 antibody to CD73-expressing MDA-MB-231 breast cancer cells.

FIG. 16 shows the blockade of CD73 enzymatic activity by anti-CD73 antibody.

FIGS. 17A-17C show the screening of affinity-maturated clones derived from anti-CD73 clone 3-1-28 antibody. FIG. 17A illustrates CD73 binding activities of phage clones. FIG. 17B illustrates binding activities of antibodies converted from phage clones numbered in FIG. 17A. FIG. 17C illustrates binding kinetics of antibodies selected in FIG. 17B.

FIGS. 18A-18B show the blockade of membrane-bound and soluble CD73 enzymatic activity by affinity-maturated anti-CD73 #3-1-28 antibodies. FIG. 18A shows membrane-bound CD73 expressed by MDA-MB-231 cells. FIG. 18B shows soluble CD73 secreted by MDA-MB-231 cells.

FIG. 19 shows the symmetric format of an anti-CD73-CD137 bispecific antibody (bsAb).

FIGS. 20A-20B show the protein aggregation and integrity of anti-CD73-CD137 bsAb by SEC-HPLC and μCE-SDS-PAGE, respectively. FIG. 20A illustrates the analysis of protein aggregation by SEC-HPLC. FIG. 20B illustrates Analysis of protein integrity by μCE-SDS-PAGE under non-reducing and reducing conditions.

FIGS. 21A-21B show the binding affinity of anti-CD73-CD137 bsAb against CD73 and CD137 antigen, respectively measured by biolayer interferometry. FIG. 21A shows binding kinetics against human CD73 antigen. FIG. 21B shows binding kinetics against human CD137 antigen.

FIG. 22 shows the simultaneous binding of CD73 and CD137 antigens by anti-CD73-CD137 bsAb.

FIG. 23 shows the blockade of CD73 enzymatic activity by anti-CD73-CD137 bsAb. Anti-CD73-CD137 bsAb showed a comparable efficacy to inhibit CD73 enzymatic activity to parental anti-CD73 antibody.

FIG. 24 shows the rescue of AMP-suppressed T-cell activation by anti-CD73-CD137 bsAb.

FIGS. 25A-25C show that anti-CD73-CD137 bsAb induces IFN-γ production of T-cells upon coculturing with CD73-expressing cancer cells. FIG. 25A illustrates the expression level of CD73 on MDA-MB-231 and NCI-H292 cells. FIG. 25B illustrates IFN-γ production of T-cells upon coculturing with CD73-expressing MDA-MB-231, breast cancer cells. FIG. 25C illustrates IFN-γ production of T-cells upon coculturing with CD73-expressing NCI-H292, lung cancer cells.

FIGS. 26A-26E show the anti-tumor activity of anti-CD73-CD137 bsAb by MDA-MB-231 breast cancer model in vivo. FIG. 26A illustrates tumor volumes. FIG. 26B illustrates tumor mass. FIG. 26C illustrates percent of alive huCD45+ cells. FIG. 26D illustrates percent of CD8+ T cells among CD3+ cells. FIG. 26E illustrates percent of CD8+ T cells among CD4+ cells.

FIG. 27 shows the pharmacokinetic profiles of anti-CD73-CD137 bsAb in vivo.

### DETAILED DESCRIPTION OF THE INVENTION

The present innovation is based on the discovery of anti-p95HER2 antibodies that show a superior activity to bind p95HER2-expressing cancer cells and of anti-CD73 antibodies that show a superior activity to neutralize the enzymatic activity of CD73 in the cell membrane-bound or soluble form. The present invention is further based on the discovery that bispecific antibodies targeting p95HER2 and CD137 possess dual functions, antibody antibody-dependent cell-mediated cytotoxicity (ADCC) and p95HER2-dependent T-cell activation, against p95HER-positive cancer cells, and that bispecific antibodies targeting CD73 and CD137 possess extraordinary activity to boost T-cell effector function and inhibit tumor growth in vivo better than mono-treatment or combination treatment with each antibody.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

p95HER2 Targeting Peptides

The receptor tyrosine kinase HER2 (ErbB2) is one of four members (HER1-4) of epidermal growth factor receptor family and overexpressed in different tumors, including 20˜30% of breast and gastric cancers. HER1-4 share a similar structure: an extracellular ligand-binding domain, a short hydrophobic transmembrane region and a cytoplasmic tyrosine kinase domain. Activation HER family through hetero/homodimerization of receptors, induced by ligand binding or receptor overexpression and result in cell proliferation, differentiation, adhesion, migration and survival.

A recombinant humanized monoclonal antibody, Trastuzumab, binds to the extracellular domain of HER2. Trastuzumab improves the relapse-free survival and overall survival in HER2-positive early breast cancer patients. Moreover, combination therapy of Trastuzumab and chemotherapy improves the survival and provides substantial clinical benefits in the HER2-positive advanced breast cancer patients. However, only 20-30% of the HER2-positive breast cancer patients respond to Trastuzumab when given as a monotherapy. The patients that do not response to Trastuzumab is caused by several potential mechanisms which lead to primary or acquired resistance, including activation the signaling of insulin-like growth factor receptor (IGF-1R), inactivation or loss the gene of phosphatase and tensin homolog deleted on chromosome 10 (PTEN) and accumulation of p95HER2.

p95HER2 is the truncated forms of HER2 receptor which lack the extracellular trastuzumab-binding domain and about 30˜40% of HER2-positive tumors express p95HER2. In some tumors, p95HER2 are the predominant HER2 forms. p95HER2 was generated by alternative initiation of translation from two methionine residues (611 and 687) or by proteolytic shedding of full-length HER2. The biologic role of p95HER2 has not been fully evaluated and the possible relationship between p95HER2 and full-length HER2 are unknown. p95HER2 possesses tyrosine kinase activity, which is essential for xenograft tumor growth in nude mice. Due to its exclusive expression in cancerous tissues, p95HER2 would be a good target for cancer therapy. A bispecific antibody that engages T cells and p95HER2-expressing cancer cells was developed and showed an effective antitumor activity in orthotopic MCF-7 breast cancer and PDX models. In addition, they also developed p95HER2 CAR T cells with remarkable activities against p95HER2-expressing primary tumor and metastasis in the lung or brain in vivo.

Bispecific antibodies (bsAbs), Blinatumomab (Blincyto®) and Catumaxomab (Removab®), that engage TAA of tumor cells and CD3 of T cells have been demonstrated anticancer efficacy and approved for the treatment of Philadelphia chromosome-negative B-acute lymphoblastic leukemia (B-ALL) and malignant ascites with ovarian cancer. However, the poor efficacy in solid tumors and systemic cytokine release of BiTE limit their usages. Compared to anti-CD3, anti-CD137 agonistic antibody may provide the better therapeutic window and safety profile as the proven efficacy against solid tumors in clinical trials and the crosslinking-dependent agonistic activity. Take advantage of cancer-specific p95HER2 expression and our invented target-dependent T-cell activation platform of crosslinking-dependent anti-CD137 single-chain antibody, it may generate a drug with potent efficacy against p95HER2-positive solid tumors meanwhile with a satisfied safety profile.

Provided here are novel anti-p95HER2 monoclonal antibodies that were generated and screened for the most potent binding affinity. Also provided herein are novel bispecific antibodies that simultaneously targeting p95HER2 and CD137, to induce a superior T-cell activation in the presence of p95HER2.

### Anti-p95HER2 Antibodies and Antigen Binding Fragments Thereof

In one embodiment, the invention provides antibodies and antigen binding fragments thereof that bind p95HER2. In one aspect, the invention provides four antibodies or antigen binding fragments thereof: anti-p95HER2 antibody clone R4-3 (p95HER2 #R4-3), anti-p95HER2 antibody clone R4-5 (p95HER2 #R4-5), anti-p95HER2 antibody clone R4-11 (p95HER2 #R4-11), anti-p95HER2 antibody clone R4-15 (p95HER2 #R4-15), anti-p95HER2 antibody clone R4-38 (p95HER2 #R4-38). In one aspect, the anti-p95HER2 antibodies provided herein include a heavy chain variable (VH) region including an amino acid sequence with at least 80% sequence identity to a sequence selected from SEQ ID NO:1, SEQ ID NO:9, SEQ ID NO:17, SEQ ID NO:25 and SEQ ID NO:33; and a light chain variable (VL) region including an amino acid sequence with at least 80% sequence identity to a sequence selected from SEQ ID NO:2, SEQ ID NO: 10, SEQ ID NO: 18, SEQ ID NO:26 and SEQ ID NO:34.

As used herein, the term “antibody” refers to an immunoglobulin molecule that has the ability to specifically bind to an antigen. The term “antibody” includes, but is not limited to, monoclonal antibodies, polyclonal antibodies, human antibodies, humanized antibodies, chimeric antibodies, bispecific antibodies, and anti-idiotypic antibodies, unless context clearly indicates otherwise. In one aspect, antibodies provided herein include monoclonal antibodies. Antibodies provided herein include any isotype and class (e.g., IgG, IgE, IgM, IgD, IgA and IgY), or subclass (e.g., IgG1, IgG2, IgG3, IgG4, IgA1 and IgA2). As used herein, “antigen binding fragment” means a fragment or portion of an immunoglobulin molecule or antibody that has the ability to specifically bind to the same antigen as the immunoglobulin molecule or antibody. Exemplary antigen binding fragments include scFv, Fab, or F(ab)2 fragments. As used herein, “antigen binding region” means the part of an antibody or immunoglobulin molecule that binds to antigens or proteins by contacting the antigen or protein, for example. An antigen binding region generally includes heavy chain variable (VH) regions and light chain variable (VL) regions. An antigen binding region generally includes one or more antigen binding sites or paratopes.

Antibodies provided herein have a VH region including an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to a sequence of SEQ ID NO: 1; SEQ ID NO:9; SEQ ID NO:17; SEQ ID NO:25 or SEQ ID NO:33. Antibodies provided herein also include an VL region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to a sequence of SEQ ID NO:2; SEQ ID NO:10; SEQ ID NO:18; SEQ ID NO:26 SEQ ID NO:34.

In general, “sequence identity” or “sequence homology,” which can be used interchangeably, refer to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby or the amino acid sequence of a polypeptide and comparing these sequences to a second nucleotide or amino acid sequence. As used herein, the term “percent (%) sequence identity” or “percent (%) identity,” also including “homology,” refers to the percentage of amino acid residues or nucleotides in a sequence that are identical with the amino acid residues or nucleotides in a reference sequence after aligning the sequences and introducing gaps, if necessary, to achieve the maximum percent sequence identity, and not considering any conservative substitutions as part of the sequence identity. Thus, two or more sequences (polynucleotide or amino acid) can be compared by determining their “percent identity,” also referred to as “percent homology.” The percent identity to a reference sequence (e.g., nucleic acid or amino acid sequences), which may be a sequence within a longer molecule (e.g., polynucleotide or polypeptide), may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin and Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values in between. Percent identities between a reference sequence and a claimed sequence can be at least 80%, at least 85%, at least 90%, at least 95%, at least 98%, at least 99%, at least 99.5%, or at least 99.9%. In general, an exact match indicates 100% identity over the length of the reference sequence. Additional programs and methods for comparing sequences and/or assessing sequence identity include the Needleman-Wunsch algorithm (see, e.g., the EMBOSS Needle aligner available at www.ebi.ac.uk/Tools/psa/emboss_needle/, optionally with default settings), the Smith-Waterman algorithm (see, e.g., the EMBOSS Water aligner available at www.ebi.ac.uk/Tools/psa/emboss_water/, optionally with default settings), the similarity search method of Pearson and Lipman, 1988, Proc. Natl. Acad. Sci. USA 85, 2444, or computer programs which use these algorithms (GAP, BESTFIT, FASTA, BLAST P, BLAST N and TFASTA in Wisconsin Genetics Software Package, Genetics Computer Group, 575 Science Drive, Madison, Wis.). In some aspects, reference to percent sequence identity refers to sequence identity as measured using BLAST (Basic Local Alignment Search Tool). In other aspects, ClustalW is used for multiple sequence alignment. Optimal alignment may be assessed using any suitable parameters of a chosen algorithm, including default parameters.

In one aspect, the antibody or antigen binding fragment thereof has a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:1 and a VL region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:2. SEQ ID NO:1 provides an amino acid sequence that includes the variable region of anti-p95HER2 antibody clone #4-3 heavy chain. SEQ ID NO: 2 provides an amino acid sequence that includes the variable region of anti-p95HER2 clone #4-3 light chain.

The antigen binding region of an antibody or antigen binding fragment thereof generally includes complementarity determining regions (CDRs). “Complementarity determining region (CDR)” refers to hypervariable regions of VH and VL. CDRs include the target protein or antigen binding site of an antibody that confers specificity for protein or antigen binding. VH and VL generally include three sequentially numbered CDRs. As used herein, CDR-H1, CDR-H2, and CDR-H3 refer to three consecutively arranged CDRs of the heavy chain variable region (VH), as numbered from the N-terminus of the heavy chain polypeptide. As used herein, CDR-L1, CDR-L2, and CDR-L3 refer to three consecutively arranged CDRs of the light chain variable region (VL), as numbered from the N-terminus of the light chain polypeptide.

In one aspect, the antigen binding region of the antibody or antigen binding fragment thereof having a VH region that includes an amino acid sequence having at least about 80% identity to SEQ ID NO: 1 and a VL region that includes an amino acid sequence having at least about 80% identity to SEQ ID NO:2 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:3, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:4, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:5. In another aspect, the antigen binding region of the antibody or antigen binding fragment thereof having a VH region that includes an amino acid sequence having at least about 80% identity to SEQ ID NO: 1 and a VL region that includes an amino acid sequence having at least about 80% identity to SEQ ID NO:2 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:6, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO: 7, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:8.

In some aspects, the antibody or antigen binding fragment thereof includes a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:9 and a VL region that includes an amino acid sequence having at least about 80%, identity at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO: 10. SEQ ID NO: 9 provides an amino acid sequence that includes the variable region of anti-p95HER2 antibody clone #4-5 heavy chain. SEQ ID NO: 10 provides an amino acid sequence that includes the variable region of anti-p95HER2 clone #4-5 light chain.

In one aspect, the antigen binding region of the antibody or antigen binding fragment thereof that includes a VH region including an amino acid sequence having at least about 80% identity to SEQ ID NO:9 and a VL region including an amino acid sequence having at least about 80% identity to SEQ ID NO:10 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:11, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:12, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:13. In another aspect, the antigen binding region of the antibody or antigen binding fragment thereof that includes a VH region including an amino acid sequence having at least about 80% identity to SEQ ID NO:9 and a VL region that includes an amino acid sequence having at least about 80%, identity to SEQ ID NO:10 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO: 14, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:15, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:16.

In some aspects, the antibody or antigen binding fragment thereof has a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:17 and a VL region that includes an amino acid sequence having at least about 80%, identity at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:18. SEQ ID NO:17 provides an amino acid sequence that includes the variable region of anti-p95HER2 antibody clone #4-11 heavy chain. SEQ ID NO: 18 provides an amino acid sequence that includes the variable region of anti-p95HER2 antibody clone #4-11 light chain.

In one aspect, the antigen binding region of the antibody or antigen binding fragment thereof that includes a VH region including an amino acid sequence having at least about 80% identity to SEQ ID NO: 17 and a VL region including an amino acid sequence having at least about 80% identity to SEQ ID NO:18 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:19, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:20, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:21. In another aspect, the antigen binding region of the antibody or antigen binding fragment thereof that includes a VH region including an amino acid sequence having at least about 80% identity to SEQ ID NO:17 and a VL region including an amino acid sequence having at least about 80%, identity to SEQ ID NO: 18 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:22, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:23, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:24.

In some aspects, the antibody or antigen binding fragment thereof has a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:25 and a VL region that includes an amino acid sequence having at least about 80%, identity at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:26. SEQ ID NO:25 provides an amino acid sequence that includes the variable region of anti-p95HER2 antibody clone #4-15 heavy chain. SEQ ID NO: 26 provides an amino acid sequence that includes the variable region of anti-p95HER2 antibody clone #4-15 light chain.

In one aspect, the antigen binding region of the antibody or antigen binding fragment thereof that includes a VH region including an amino acid sequence having at least about 80% identity to SEQ ID NO:25 and a VL region including an amino acid sequence having at least about 80% identity to SEQ ID NO:26 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:27, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:28, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:29. In another aspect, the antigen binding region of the antibody or antigen binding fragment thereof that includes a VH region including an amino acid sequence having at least about 80% identity to SEQ ID NO:25 and a VL region including an amino acid sequence having at least about 80%, identity to SEQ ID NO:26 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:30, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:31, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:32.

In some aspects, the antibody or antigen binding fragment thereof has a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:33 and a VL region that includes an amino acid sequence having at least about 80%, identity at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:34. SEQ ID NO:33 provides an amino acid sequence that includes the variable region of anti-p95HER2 antibody clone #4-38 heavy chain. SEQ ID NO: 34 provides an amino acid sequence that includes the variable region of anti-p95HER2 antibody clone #4-38 light chain.

In one aspect, the antigen binding region of the antibody or antigen binding fragment thereof that includes a VH region including an amino acid sequence having at least about 80% identity to SEQ ID NO:33 and a VL region including an amino acid sequence having at least about 80% identity to SEQ ID NO:34 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:35, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:36, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:37. In another aspect, the antigen binding region of the antibody or antigen binding fragment thereof that includes a VH region including an amino acid sequence having at least about 80% identity to SEQ ID NO:33 and a VL region including an amino acid sequence having at least about 80%, identity to SEQ ID NO:34 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:38, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:39, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:40.

Antibodies or antigen binding fragments thereof provided herein further include an Fc domain. As used herein, the term Fc domain refers to an antibody region that includes at least a hinge region, a CH2 domain, and a CH3 domain, unless context clearly indicates otherwise. The terms Fc domain and Fc region may be used interchangeably, unless context clearly indicates otherwise. In certain aspects, the Fc domain is an IgG domain, an IgE domain, an IgM domain, and IgD domain, an IgA domain, or an IgY domain. Fc domains of any sequence and from any species can be used, including human, ape, monkey, mouse, rabbit, goat, sheep, guinea pig, horse, and others. In certain aspects, Fc domains are engineered, i.e., non-naturally occurring or recombinant Fc domains generated using techniques of molecular biology, for example. In some aspects, the IgG domain is an IgG1 domain, an IgG2 domain, an IgG3 domain, or an IgG4 domain.

### Anti-p95HER2 Bispecific Antibodies

Bispecific molecules, such as bispecific antibodies (bsAbs), provide a means for simultaneously targeting multiple epitopes on the same or different molecular targets with a single therapeutic agent. Without being limited by theory, bispecific molecules as cancer therapeutics have the potential to confer novel or more potent activities, lower the cost of goods, and facilitate the development of new therapeutic regimens as compared to a mixture of two monoclonal antibodies (mAbs), for example.

Provided herein, in some embodiments, are bispecific antibodies that include a first antigen binding region and a second antigen binding region. Generally, the first antigen binding region and the second antigen binding region specifically bind to different antigens or targets. In some aspects, the first antigen binding region and the second antigen binding region bind to different epitopes in the same antigen or target.

In an embodiment, the invention provides bispecific antibodies including a first antigen binding region and a second antigen binding region, wherein the first antigen binding region binds to p95HER2. The first antigen binding region includes a VH region that includes an amino acid sequence having at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, and any number or range in between, to a sequence selected from SEQ ID NO:17 and a VL region that includes an amino acid sequence having at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, and any number or range in between, to about 100 to 120 amino acids of an N-terminal sequence of a sequence selected from SEQ ID NO:18; In some aspects, the second antigen binding region of bispecific antibodies provided herein binds to an immune checkpoint molecule, an immune stimulatory molecule, or a tumor antigen.

In one aspect, the bispecific antibody includes a V_{H} region including complementarity-determining region (CDR)-H1, CDR-H2, and CDR-H3, wherein CDR-H1 is set forth in SEQ ID NO:19, CDR-H2 is set forth in SEQ ID NO:20, and CDR-H3 is set forth in SEQ ID NO:21 or sequences having 90% identity to SEQ ID NO: 19, 20 or 21 and the antigen binding specificity thereof; and a V_{L} region including CDR-L1, CDR-L2, and CDR-L3, wherein CDR-L1 is set forth in SEQ ID NO:22, CDR-L2 is set forth in SEQ ID NO:23, and CDR-L3 is set forth in SEQ ID NO:24 or sequences having 90% identity to SEQ ID NO: 19, 20 or 21 and the antigen binding specificity thereof. In another aspect, the VH region has an amino acid sequence having at least 80% identity to SEQ ID NO:17 and an antigen binding specificity of SEQ ID NOs: 19-21 and the VL region has an amino acid sequence having at least 80% identity to SEQ ID NO: 18 and an antigen binding specificity of SEQ ID NOs: 22-24.

Any combination of first and second antigen binding regions can be included in bispecific antibodies provided herein, including first and second antigen binding regions that bind to any immune checkpoint molecule, any immune stimulatory molecule, or any tumor antigen, for example. Accordingly, in some aspects, the second antigen binding region of bispecific antibodies provided herein binds to any immune checkpoint molecule, any immune stimulatory molecule, or any tumor antigen. In some aspects, the first and second antigen binding regions bind to the same molecule. For example, the first and second antigen binding regions may bind to the same or to a different epitope of the same molecule. In other aspects, the first and second antigen binding regions bind to different molecules.

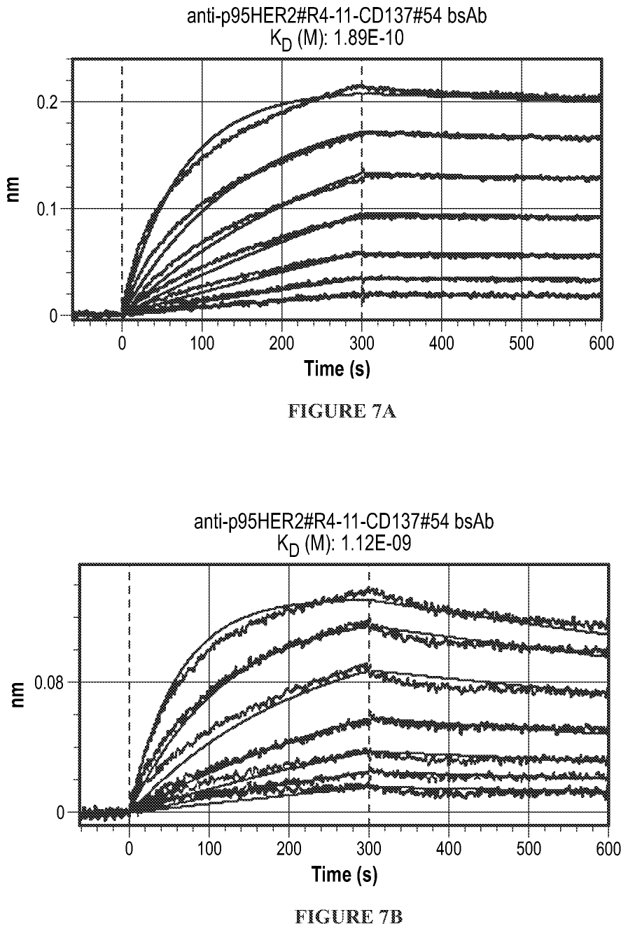

In one aspect, bispecific antibodies having a first antigen binding region that binds to p95HER2 and a second antigen binding region that binds to CD137 bind to p95HER2 and CD137 simultaneously (FIG. 8). Without being limited by theory, restricting anti-p95HER2 binding activity to tumor sites that express p95HER2 by designing bispecific antibodies that are able to bind to both p95HER2 and CD137 may reduce the risk of hepatotoxicity and its associated fatality seen in clinical trials with anti-CD137 antibodies such as Urelumab. In addition, it is believed that simultaneous binding to p95HER2 and CD137 may enhance T-cell activation as a result of cross-linking (see also Example 8 below).

In some aspects, first antigen binding regions and second antigen binding regions include an scFv, an F(ab)2, an Fab, or any combination thereof. In one aspect, the first antigen binding region includes an scFv and the second antigen binding region includes an Fab. In another aspect, an scFv included in bispecific antibodies provided herein binds to an immune checkpoint molecule, an immune stimulatory molecule, or a tumor antigen. In yet another aspect, an scFv included in bispecific antibodies provided herein binds to a CD137. In some aspects, an Fab included in bispecific antibodies provided herein binds to an immune checkpoint molecule, an immune stimulatory molecule, or a tumor antigen. In one aspect, an Fab included in bispecific antibodies provided herein binds to p95HER2. In another aspect, the scFv of bispecific antibodies provided herein includes an amino acid sequence of SEQ ID NO: 48.

In some aspects, bispecific antibodies provided herein further include a linker between the VH region and the VL region of the scFv. Any linker can be used. For example, linkers can include any amino acid sequence. Linkers can be of any length, such as one amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, or more amino acids. Linkers can also include multiples of an amino acid sequence. Any number of multiples of an amino acid sequence can be included in a linker. Exemplary linker sequences are provided in SEQ ID NO:44, SEQ ID NO: 45 and SEQ ID NO:160.

In some aspects, bispecific antibodies provided herein include a heavy chain sequence of SEQ ID NO:46. In another aspect, bispecific antibodies provided herein further include a light chain sequence of SEQ ID NO:18. Heavy chain sequences such as SEQ ID NO: 46, and others, can include one or more G linkers (SEQ ID NO:44), one or more G4S linkers (SEQ ID NO:45 or SEQ ID NO:160), or any multiple of a G linker or G4S linker, although any other suitable linker can be used.

### CD73 Targeting Peptides

Immunoregulation of the adaptive immune system has been an attractive field in cancer immunotherapy because of fewer side effects and long-term protection from cancer relapse. Despite the success of anti-PD-1/PD-L1 therapies in advanced cancer, a considerable proportion of patients remain unresponsive to these treatments. This represents that other immunosuppressive mechanisms coexist in the tumor microenvironment, and the adenosinergic pathway has emerged as a key player in it.

Tumor hypoxia, chronic inflammation, and metabolic changes give rise to extracellular ATP accumulation that promote anti-tumor activity through inductions of inflammasome assembly and proinflammatory cytokines secretion. However, owing to the presence of hypoxia, inflammatory cytokines, and TGF-β, higher levels of ectonucleotidases CD39 and CD73 usually observed in solid tumors compared to the corresponding non-malignant tissues. Extracellular accumulated ATP was sequentially hydrolyzed by tumor associated CD39 and CD73 to adenosine, then mediates it regulatory functions upon binding to adenosine receptors expressed by tumor cells, stromal cells, and infiltrating lymphocytes in tumors.

In tumor cells, adenosine signaling via A2A and A2B receptors promotes proliferation, metastasis, and secretion of vascular endothelial growth factor (VEGF). In cancer-associated fibroblasts (CAFs), adenosine signaling via A2BR induces the expressions of pro-tumor fibroblast growth factor 2 (FGF2) and CXCL12 that create a pro-tumor microenvironment. In the tumor-infiltrating immune cells, A2A signaling inhibits effector T cell activation through dampening proximal TCR signaling and concurrently inducing co-inhibitory molecules PD-1, LAG3, and TIM3 expression. In addition, A2A signaling promotes differentiation, proliferation, and CTLA-4 expression of murine Treg cells. In NK cells, A2A signaling blocks maturation and cytotoxicity. In professional antigen-presenting cells, macrophage and dendritic cells, A2A and A2B signaling downregulate the expressions of antigen presentation and costimulatory molecules, while promotes M2 macrophage differentiation.

High levels of CD73 were found in the malignant tissues of various cancer types, including pancreatic, gastrointestinal, renal and lung cancers, and corresponded to a poor prognosis. High levels of soluble CD73 were also observed in the circulation of patients with metastatic melanoma. Given the pro-tumor functions of adenosine and relevance of CD73 in cancer prognosis, CD73 has become an attractive therapeutic target for cancers. In the preclinical studies, antitumor activities of small molecule inhibitors and antibodies target CD73 were attributed not only to the inhibition of tumor cell growth and migration, but the involvement of adaptive immunity as well. In addition, combine anti-CD73 antibody enhanced antitumor activities of antibodies against other immune regulatory molecules, including CTLA-4, PD-1, and CD137. The synergized antitumor activity in the combination with anti-PD-1 antibody was attribute to the activation of A2A signaling that enhance PD-1 expression on tumor-specific CD8+T. Intriguingly, tumor resistance to anti-CD137 antibody therapy was referred to the upregulation of CD73 on tumor-infiltrating CD8+ T cells, therefore a more effective antitumor activity was elicited by the combination therapy of anti-CD137 and CD73 antibody.

In clinical studies, several anti-CD73 antibodies including, MEDI9447 (oleclumab), CPI-006, BMS-986179, and NZV930, are undergoing Phase I/II in the monotherapy or combinational therapies. The partial response was observed in 4.8% (1/21) patients with colon rectal cancer, and in 10% (2/20) patients with pancreatic cancer by treating with oleclumab and durvalumab (anti-PD-L1 antibody). The partial response was observed in 13.5% (7/52) patients with head and neck, pancreatic, prostate, anal, and renal cancer by the combination of BMS-986179 and nivolumab (anti-PD-1 antibody).

Provided herein are novel anti-CD73 monoclonal antibodies that were generated and screened for the most potent binding affinity and enzymatic blockade activity. Also provided herein are a novel bispecific antibody that simultaneously targeting CD73 and CD137 or PD-L1, to induce a superior T-cell activation upon coculturing with CD73+ tumor cells and antitumor activity in vivo.

### Anti-CD73 Antibodies and Antigen Binding Fragments Thereof

In one embodiment, the invention provides antibodies and antigen binding fragments thereof that bind CD73. In one aspect, the invention provides four antibodies or antigen binding fragments thereof: anti-CD73 antibody clone 3-4 (CD73 #3-4), anti-CD73 antibody clone 3-16 (CD73 #3-16), anti-CD73 antibody clone 3-1-28 (CD73 #3-1-28), and anti-CD73 antibody clone 3-1-37 (CD73 #3-1-37). In another aspect, the invention provides three affinity-maturated antibodies derived from anti-CD73 clone 3-1-28: anti-CD73 #3-1-28 (S51A Q54E), anti-CD73 #3-1-28 (S107D S109H), anti-CD73 #3-1-28 (S107D S109T A114S).

In one aspect, the anti-CD73 antibodies provided herein include a heavy chain variable (VH) region including an amino acid sequence with at least 80% sequence identity to a sequence selected from SEQ ID NO:49, SEQ ID NO:57, SEQ ID NO:65 and SEQ ID NO:73; and a light chain variable (VL) region including an amino acid sequence with at least 80% sequence identity to a sequence selected from SEQ ID NO:50, SEQ ID NO:58, SEQ ID NO:66, SEQ ID NO:74, SEQ ID NO:81, SEQ ID NO:83 and SEQ ID NO:85.

Antibodies provided herein have a VH region including an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to a sequence of SEQ ID NO: 49, SEQ ID NO:57, SEQ ID NO:65, and SEQ ID NO:73. Antibodies provided herein also include an VL region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to a sequence of SEQ ID NO:50, SEQ ID NO:58, SEQ ID NO:66 and SEQ ID NO:74, SEQ ID NO:81, SEQ ID NO:83 and SEQ ID NO:85.

In one aspect, the antibody or antigen binding fragment thereof has a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:49 and a VL region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:50. SEQ ID NO:49 provides an amino acid sequence that includes the variable region of anti-CD73 antibody clone #3-4 heavy chain. SEQ ID NO: 50 provides an amino acid sequence that includes the variable region of anti-CD73 clone #3-4 light chain.

In one aspect, the antigen binding region of the antibody or antigen binding fragment thereof having a VH region that includes an amino acid sequence having at least about 80% identity to SEQ ID NO:49 and a VL region that includes an amino acid sequence having at least about 80% identity to SEQ ID NO:50 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:51, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:52, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:53. In another aspect, the antigen binding region of the antibody or antigen binding fragment thereof having a VH region that includes an amino acid sequence having at least about 80% identity to SEQ ID NO:49 and a VL region that includes an amino acid sequence having at least about 80% identity to SEQ ID NO:50 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:54, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:55, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:56.

In some aspects, the antibody or antigen binding fragment thereof includes a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:57 and a VL region that includes an amino acid sequence having at least about 80%, identity at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:58. SEQ ID NO: 57 provides an amino acid sequence that includes the variable region of anti-CD73 antibody clone #3-16 heavy chain. SEQ ID NO:58 provides an amino acid sequence that includes the variable region of anti-CD73 clone #3-16 light chain.

In one aspect, the antigen binding region of the antibody or antigen binding fragment thereof that includes a VH region including an amino acid sequence having at least about 80% identity to SEQ ID NO:57 and a VL region including an amino acid sequence having at least about 80% identity to SEQ ID NO:58 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:59, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:60, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:61. In another aspect, the antigen binding region of the antibody or antigen binding fragment thereof that includes a VH region including an amino acid sequence having at least about 80% identity to SEQ ID NO:57 and a VL region that includes an amino acid sequence having at least about 80%, identity to SEQ ID NO:58 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:62, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:63, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:64.

In some aspects, the antibody or antigen binding fragment thereof has a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:65 and a VL region that includes an amino acid sequence having at least about 80%, identity at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:66. SEQ ID NO:65 provides an amino acid sequence that includes the variable region of anti-CD73 antibody clone #3-1-28 heavy chain. SEQ ID NO:66 provides an amino acid sequence that includes the variable region of anti-CD73 clone #3-1-28 light chain.

In one aspect, the antigen binding region of the antibody or antigen binding fragment thereof that includes a VH region including an amino acid sequence having at least about 80% identity to SEQ ID NO:65 and a VL region including an amino acid sequence having at least about 80% identity to SEQ ID NO:66 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:67, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:68, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:69. In another aspect, the antigen binding region of the antibody or antigen binding fragment thereof that includes a VH region including an amino acid sequence having at least about 80% identity to SEQ ID NO:65 and a VL region including an amino acid sequence having at least about 80%, identity to SEQ ID NO:66 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO: 70, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:71, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:72.

In some aspects, the antibody or antigen binding fragment thereof has a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:73 and a VL region that includes an amino acid sequence having at least about 80%, identity at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:74. SEQ ID NO:73 provides an amino acid sequence that includes the variable region of anti-CD73 antibody clone #3-1-37 heavy chain. SEQ ID NO:74 provides an amino acid sequence that includes the variable region of anti-CD73 clone #3-1-37 light chain.

In one aspect, the antigen binding region of the antibody or antigen binding fragment thereof that includes a VH region including an amino acid sequence having at least about 80% identity to SEQ ID NO:73 and a VL region including an amino acid sequence having at least about 80% identity to SEQ ID NO:74 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:75, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:76, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:77. In another aspect, the antigen binding region of the antibody or antigen binding fragment thereof that includes a VH region including an amino acid sequence having at least about 80% identity to SEQ ID NO:73 and a VL region including an amino acid sequence having at least about 80%, identity to SEQ ID NO:74 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:78, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:79, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:80.

In some aspects, the antibody or antigen binding fragment thereof has a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:65 and a VL region that includes an amino acid sequence having at least about 80%, identity at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:81. SEQ ID NO:65 provides an amino acid sequence that includes the variable region of anti-CD73 antibody clone #3-1-28 heavy chain. SEQ ID NO:81 provides an amino acid sequence that includes the variable region of anti-CD73 clone #3-1-28 (S51A Q54E) light chain.

In one aspect, the antigen binding region of the antibody or antigen binding fragment thereof that includes a VH region including an amino acid sequence having at least about 80% identity to SEQ ID NO:65 and a VL region including an amino acid sequence having at least about 80% identity to SEQ ID NO:81 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:67, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:68, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:69. In another aspect, the antigen binding region of the antibody or antigen binding fragment thereof that includes a VH region including an amino acid sequence having at least about 80% identity to SEQ ID NO:65 and a VL region including an amino acid sequence having at least about 80%, identity to SEQ ID NO:81 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:70, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:82, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:72.

In some aspects, the antibody or antigen binding fragment thereof has a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:65 and a VL region that includes an amino acid sequence having at least about 80%, identity at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:83. SEQ ID NO:65 provides an amino acid sequence that includes the variable region of anti-CD73 antibody clone #3-1-28 heavy chain. SEQ ID NO:83 provides an amino acid sequence that includes the variable region of anti-CD73 clone #3-1-28 (S107D S109H) light chain.

In one aspect, the antigen binding region of the antibody or antigen binding fragment thereof that includes a VH region including an amino acid sequence having at least about 80% identity to SEQ ID NO:65 and a VL region including an amino acid sequence having at least about 80% identity to SEQ ID NO:83 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:67, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:68, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:69. In another aspect, the antigen binding region of the antibody or antigen binding fragment thereof that includes a VH region including an amino acid sequence having at least about 80% identity to SEQ ID NO:65 and a VL region including an amino acid sequence having at least about 80%, identity to SEQ ID NO:83 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:70, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:84, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:72.

In some aspects, the antibody or antigen binding fragment thereof has a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:65 and a VL region that includes an amino acid sequence having at least about 80%, identity at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:85. SEQ ID NO:65 provides an amino acid sequence that includes the variable region of anti-CD73 antibody clone #3-1-28 heavy chain. SEQ ID NO:85 provides an amino acid sequence that includes the variable region of anti-CD73 clone #3-1-28 (S107D S109T A114S) light chain.

In one aspect, the antigen binding region of the antibody or antigen binding fragment thereof that includes a VH region including an amino acid sequence having at least about 80% identity to SEQ ID NO:65 and a VL region including an amino acid sequence having at least about 80% identity to SEQ ID NO:85 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:67, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:68, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:69. In another aspect, the antigen binding region of the antibody or antigen binding fragment thereof that includes a VH region including an amino acid sequence having at least about 80% identity to SEQ ID NO:65 and a VL region including an amino acid sequence having at least about 80%, identity to SEQ ID NO:85 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:70, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:86, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:72.

Antibodies or antigen binding fragments thereof provided herein further include an Fc domain. In certain aspects, the Fc domain is an IgG domain, an IgE domain, an IgM domain, and IgD domain, an IgA domain, or an IgY domain. In some aspects, the IgG domain is an IgG1 domain, an IgG2 domain, an IgG3 domain, or an IgG4 domain.

### Anti-CD73 Bispecific Antibodies

Provided herein are bi-functional proteins, including bispecific antibodies. As used herein, the term “bi-functional protein” refers to a protein that has at least two functions. A non-limiting example of a bi-functional protein includes a bispecific antibody that is capable of binding to two antigens. Bispecific antibodies provided herein can include isolated, functional scFv fragments that bind to CD137 and that are fused to the C-terminus of the Fc domain of an anti-CD73 antibody, for example. In some aspects, a C-terminally positioned scFv that binds CD137 in fusion constructs provided herein is fused to an Fc domain of an antibody that binds to other immunoregulatory molecules, such as CD40 or CTLA-4, for example. In other aspects, a C-terminally positioned scFv can bind to an immunoregulatory molecule, such as CD40 or CTLA-4, for example.

In one embodiment, the invention provides a bispecific antibody including a first antigen binding region and a second antigen binding region, wherein the first antigen binding region binds to CD73. The first antigen binding region includes a VH region that includes an amino acid sequence having at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, and any number or range in between, to a sequence selected from SEQ ID NO:65 and a VL region that includes an amino acid sequence having at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, and any number or range in between, to about 100 to 120 amino acids of an N-terminal sequence of a sequence selected from SEQ ID NO:66; In some aspects, the second antigen binding region of bispecific antibodies provided herein binds to an immune checkpoint molecule, an immune stimulatory molecule, or a tumor antigen.

In one aspect, the bispecific antibody includes a V_{H} region including complementarity-determining region (CDR)-H1, CDR-H2, and CDR-H3, wherein CDR-H1 is set forth in SEQ ID NO:67, CDR-H2 is set forth in SEQ ID NO:68, and CDR-H3 is set forth in SEQ ID NO:69 or sequences having 90% identity to SEQ ID NO:67, 68 or 69 and the antigen binding specificity thereof; and a V_{L} region including CDR-L1, CDR-L2, and CDR-L3, wherein CDR-L1 is set forth in SEQ ID NO:70, CDR-L2 is set forth in SEQ ID NO:71, and CDR-L3 is set forth in SEQ ID NO:72 or sequences having 90% identity to SEQ ID NO:70, 71 or 72 and the antigen binding specificity thereof. In another aspect, the VH region has an amino acid sequence having at least 80% identity to SEQ ID NO:65 and an antigen binding specificity of SEQ ID NOs: 67-69 and the VL region has an amino acid sequence having at least 80% identity to SEQ ID NO:66 and an antigen binding specificity of SEQ ID NOs: 70-72.

The first antigen binding region includes a VH region that includes an amino acid sequence having at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, and any number or range in between, to a sequence selected from SEQ ID NO:65 and a VL region that includes an amino acid sequence having at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, and any number or range in between, to about 100 to 120 amino acids of an N-terminal sequence of a sequence selected from SEQ ID NO:83; In some aspects, the second antigen binding region of bispecific antibodies provided herein binds to an immune checkpoint molecule, an immune stimulatory molecule, or a tumor antigen.

In one aspect, the bispecific antibody includes a V_{H} region including complementarity-determining region (CDR)-H1, CDR-H2, and CDR-H3, wherein CDR-H1 is set forth in SEQ ID NO:67, CDR-H2 is set forth in SEQ ID NO:68, and CDR-H3 is set forth in SEQ ID NO:69 or sequences having 90% identity to SEQ ID NO:67, 68 or 69 and the antigen binding specificity thereof; and a V_{L} region including CDR-L1, CDR-L2, and CDR-L3, wherein CDR-L1 is set forth in SEQ ID NO:70, CDR-L2 is set forth in SEQ ID NO:84, and CDR-L3 is set forth in SEQ ID NO:72 or sequences having 90% identity to SEQ ID NO:70, 84 or 72 and the antigen binding specificity thereof. In another aspect, the VH region has an amino acid sequence having at least 80% identity to SEQ ID NO:65 and an antigen binding specificity of SEQ ID NOs: 67-69 and the VL region has an amino acid sequence having at least 80% identity to SEQ ID NO:83 and an antigen binding specificity of SEQ ID NOs: 70, 84 and 72.

The first antigen binding region includes a VH region that includes an amino acid sequence having at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, and any number or range in between, to a sequence selected from SEQ ID NO:65 and a VL region that includes an amino acid sequence having at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, and any number or range in between, to about 100 to 120 amino acids of an N-terminal sequence of a sequence selected from SEQ ID NO:85; In some aspects, the second antigen binding region of bispecific antibodies provided herein binds to an immune checkpoint molecule, an immune stimulatory molecule, or a tumor antigen.

In one aspect, the bispecific antibody includes a V_{H} region including complementarity-determining region (CDR)-H1, CDR-H2, and CDR-H3, wherein CDR-H1 is set forth in SEQ ID NO:67, CDR-H2 is set forth in SEQ ID NO:68, and CDR-H3 is set forth in SEQ ID NO:69 or sequences having 90% identity to SEQ ID NO:67, 68 or 69 and the antigen binding specificity thereof; and a V_{L} region including CDR-L1, CDR-L2, and CDR-L3, wherein CDR-L1 is set forth in SEQ ID NO:70, CDR-L2 is set forth in SEQ ID NO:85, and CDR-L3 is set forth in SEQ ID NO:72 or sequences having 90% identity to SEQ ID NO:70, 86 or 72 and the antigen binding specificity thereof. In another aspect, the VH region has an amino acid sequence having at least 80% identity to SEQ ID NO:65 and an antigen binding specificity of SEQ ID NOs: 67-69 and the VL region has an amino acid sequence having at least 80% identity to SEQ ID NO:85 and an antigen binding specificity of SEQ ID NOs: 70, 86 and 72.

In some aspects, the second antigen binding region of bispecific antibodies provided herein binds to an immune checkpoint molecule, an immune stimulatory molecule, or a tumor antigen.

Any combination of first and second antigen binding regions can be included in bispecific antibodies provided herein, including first and second antigen binding regions that bind to any immune checkpoint molecule, any immune stimulatory molecule, or any tumor antigen, for example. Accordingly, in some aspects, the second antigen binding region of bispecific antibodies provided herein binds to any immune checkpoint molecule, any immune stimulatory molecule, or any tumor antigen. In some aspects, the first and second antigen binding regions bind to the same molecule. For example, the first and second antigen binding regions may bind to the same or to a different epitope of the same molecule. In other aspects, the first and second antigen binding regions bind to different molecules.

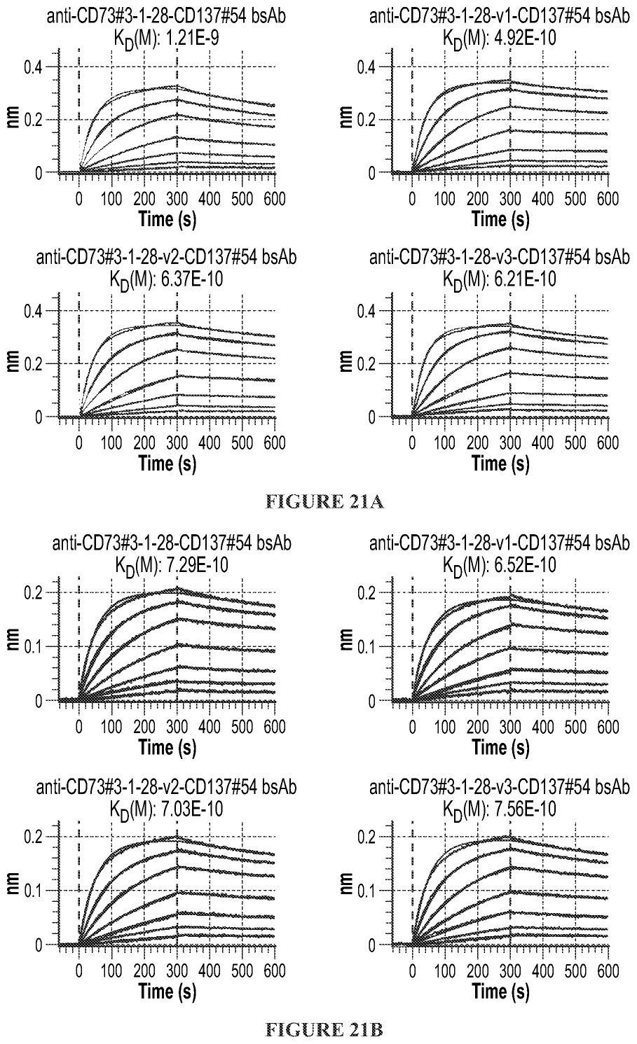

In one aspect, bispecific antibodies having a first antigen binding region that binds to CD73 and a second antigen binding region that binds to CD137 bind to CD73 and CD137 simultaneously (FIG. 22). Without being limited by theory, restricting anti-CD73 binding activity to tumor sites that express CD73 by designing bispecific antibodies that are able to bind to both CD73 and CD137 may reduce the risk of hepatotoxicity and its associated fatality seen in clinical trials with anti-CD137 antibodies such as Urelumab. In addition, it is believed that simultaneous binding to CD73 and CD137 may enhance T-cell activation as a result of cross-linking (see also Example 21 below).

In some aspects, first antigen binding regions and second antigen binding regions include an scFv, an F(ab)2, an Fab, or any combination thereof. In one aspect, the first antigen binding region includes an scFv and the second antigen binding region includes an Fab. In another aspect, an scFv included in bispecific antibodies provided herein binds to an immune checkpoint molecule, an immune stimulatory molecule, or a tumor antigen. In yet another aspect, an scFv included in bispecific antibodies provided herein binds to a CD137. In some aspects, an Fab included in bispecific antibodies provided herein binds to an immune checkpoint molecule, an immune stimulatory molecule, or a tumor antigen. In one aspect, an Fab included in bispecific antibodies provided herein binds to CD73. In one aspect, the antibody includes a heavy chain sequence having the amino acid sequence of SEQ ID NO:88, 89, 90 or 91 or a sequence having 90% identity thereto and the binding specificity thereof. In another aspect, the bispecific antibody further includes a light chain having the amino acid sequence of SEQ ID NO:66. In another aspect, the scFv of bispecific antibodies provided herein includes an amino acid sequence of SEQ ID NO:91.

In some aspects, bispecific antibodies provided herein further include a linker between the VH region and the VL region of the scFv. Any linker can be used. For example, linkers can include any amino acid sequence. Linkers can be of any length, such as one amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, or more amino acids. Linkers can also include multiples of an amino acid sequence. Any number of multiples of an amino acid sequence can be included in a linker. Exemplary linker sequences are provided in SEQ ID NO:44, SEQ ID NO: 45 and 160.

In some aspects, bispecific antibodies provided herein include a heavy chain sequence of SEQ ID NO:88, SEQ ID NO:89, or SEQ ID NO:91. In another aspect, bispecific antibodies provided herein further include a light chain sequence of SEQ ID NO:66 or SEQ ID NO: 81. Heavy chain sequences such as SEQ ID NO:88, SEQ ID NO: 89, SEQ ID NO: 91, and others, can include one or more G linkers (SEQ ID NO:44), one or more G4S linkers (SEQ ID NO: 45 and SEQ ID NO: 160), or any multiple of a G linker or G4S linker, although any other suitable linker can be used.

### Antibody-Drug Conjugates

In one embodiment, the invention provides an antibody-drug conjugate including: a therapeutic agent, and any one of the antibodies or antigen binding fragments thereof described herein, or any one of the bispecific antibodies described herein. For example, the antibody, antigen binding fragment thereof or the bispecific antibody include anti-p95HER2 antibody, antigen binding fragment thereof, bispecific antibody targeting p95HER2 and a second target, anti-CD73 antibody, antigen binding fragment thereof, and bispecific antibody targeting CD73 and a second target, such as those described herein.

The term “antibody drug conjugate” can be used interchangeably with the term “immunoconjugate” or “conjugate” as used herein and refers to a compound or a derivative thereof, such as a drug (i.e., an anti-cancer drug) that is linked to a cell binding agent (i.e., an antibody or fragment thereof, or a bispecific antibody) and is defined by a generic formula: C-L-A, wherein C=cytotoxin, L=linker, A=cell binding agent or antibody. Immunoconjugates can also be defined by the generic formula in reverse order: A-L-C.

In one aspect, the therapeutic agent is covalently linked to the antibody, antigen binding fragment or bispecific antibody via a linker.

By “therapeutic agent” it is meant any drug, compound or small molecule, useful for the treatment of cancer, that can be linked to the binding agent described herein. In various aspects, the therapeutic agent is a chemotherapeutic agent. Chemotherapeutic and antineoplastic agents are well known cytotoxic agents, and include but are not limited to: (i) anti-microtubules agents comprising vinca alkaloids (vinblastine, vincristine, vinflunine, vindesine, and vinorelbine), taxanes (cabazitaxel, docetaxel, larotaxel, ortataxel, paclitaxel, and tesetaxel), epothilones (ixabepilone), and podophyllotoxin (etoposide and teniposide); (ii) antimetabolite agents comprising anti-folates (aminopterin, methotrexate, pemetrexed, pralatrexate, and raltitrexed), and deoxynucleoside analogues (azacitidine, capecitabine, carmofur, cladribine, clofarabine, cytarabine, decitabine, doxifluridine, floxuridine, fludarabinc, fluorouracil, gemcitabine, hydroxycarbamide, mercaptopurine, nelarabine, pentostatin, tegafur, and thioguanine); (iii) topoisomerase inhibitors comprising Topoisomerase I inhibitors (belotecan, camptothecin, cositecan, gimatecan, exatecan, irinotecan, lurtotecan, silatecan, topotecan, and rubitecan) and Topoisomerase II inhibitors (aclarubicin, amrubicin, daunorubicin, doxorubicin, epirubicin, ctoposide, idarubicinm, merbarone, mitoxantrone, novobiocin, pirarubicin, teniposide, valrubicin, and zorubicin); (iv) alkylating agents comprising nitrogen mustards (bendamustine, busulfan, chlorambucil, cyclophosphamide, estramustine phosphate, ifosamide, mechlorethamine, melphalan, prednimustinc, trofosfamide, and uramustinc), nitrosoureas (carmustine (BCNU), fotemustine, lomustine (CCNU), N-Nitroso-N-methylurca (MNU), nimustine, ranimustine semustine (MeCCNU), and streptozotocin), platinum-based (cisplatin, carboplatin, dicycloplatin, nedaplatin, oxaliplatin and satraplatin), aziridines (carboquonc, thiotepa, mytomycin, diaziquone (AZQ), triaziquone and tricthylenemelamine), alkyl sulfonates (busulfan, mannosulfan, and treosulfan), non-classical alkylating agents (hydrazines, procarbazine, triazenes, hexamethylmelamine, altretamine, mitobronitol, and pipobroman), tetrazines (dacarbazine, mitozolomide and temozolomide); (v) anthracyclines agents comprising doxorubicin and daunorubicin. Derivatives of these compounds include epirubicin and idarubicin; pirarubicin, aclarubicin, and mitoxantronc, bleomycins, mitomycin C, mitoxantrone, and actinomycin; (vi) enzyme inhibitors agents comprising FI inhibitor (Tipifarnib), CDK inhibitors (Abemaciclib, Alvocidib, Palbociclib, Ribociclib, and Scliciclib), Prl inhibitor (Bortezomib, Carfilzomib, and Ixazomib), PhI inhibitor (Anagrelide), IMPDI inhibitor (Tiazofurin), LI inhibitor (Masoprocol), PARP inhibitor (Niraparib, Olaparib, Rucaparib), HDAC inhibitor (Belinostat, Panobinostat, Romidepsin, Vorinostat), and PIKI inhibitor (Idelalisib); (vii) receptor antagonist agent comprising ERA receptor antagonist (Atrasentan), Retinoid X receptor antagonist (Bexarotene), Sex steroid receptor antagonist (Testolactone); (viii) ungrouped agent comprising Amsacrine, Trabectedin, Retinoids (Alitretinoin Tretinoin) Arsenic trioxide, Asparagine depleters (Asparaginase/Pegaspargase), Celecoxib, Demecolcine Elesclomol, Elsamitrucin, Etoglucid, Lonidamine, Lucanthonc, Mitoguazone, Mitotane, Oblimersen, Omacetaxine mepesuccinate, and Eribulin.

### Pharmaceutical Compositions

In one embodiment, the invention provides a pharmaceutical composition including any one of the antibodies or antigen binding fragments thereof described herein, any one of the bispecific antibodies described herein or any one of the antibody-drug conjugates described herein, and at least one pharmaceutical acceptable carrier.

For example, the pharmaceutical composition includes an anti-p95HER2 antibody, an antigen binding fragment thereof, a bispecific antibody targeting p95HER2 and a second target, an antibody-drug conjugate thereof, an anti-CD73 antibody, an antigen binding fragment thereof, a bispecific antibody targeting CD73 and a second target, or an antibody-drug conjugate thereof, such as those described herein.

As used herein, “pharmaceutical composition” refers to a formulation comprising an active ingredient, and optionally one or more pharmaceutically acceptable carriers, diluents or excipients. The term “active ingredient” can interchangeably refer to an “effective ingredient” and is meant to refer to any agent that is capable of inducing a sought-after effect upon administration. In one embodiment, the active ingredient includes a biologically active molecule. As used herein, the phrase “biologically active molecule” refers to a molecule that has a biological effect in a cell. In certain embodiments the active molecule an antibody, an antibody fragment, a bispecific antibody or an antibody-drug conjugate, such as those described herein or a morpholino.

By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

In some aspects, the pharmaceutically acceptable carrier is conjugated to the C-terminus of one or more polypeptides of the antibody or antigen binding fragment. Any suitable means of conjugating the pharmaceutically acceptable carrier can be used, including covalent conjugation and use of linkers, for example.

### Methods of Use

In one embodiment, the invention provides a method of treating cancer including administering to a subject in need thereof an effective amount of any one of the antibodies or antigen binding fragments thereof described herein, any one of the bispecific antibodies described herein, or any one of the antibody-drug conjugates described herein, thereby treating cancer.

As used herein, the terms “treat,” “treatment,” “therapy,” “therapeutic,” and the like refer to obtaining a desired pharmacologic and/or physiologic effect, including, but not limited to, alleviating, delaying or slowing the progression, reducing the effects or symptoms, preventing onset, inhibiting, ameliorating the onset of a diseases or disorder, obtaining a beneficial or desired result with respect to a disease, disorder, or medical condition, such as a therapeutic benefit and/or a prophylactic benefit. “Treatment,” as used herein, includes any treatment of a disease in a mammal, particularly in a human, and includes: (a) preventing the disease from occurring in a subject, including a subject which is predisposed to the disease or at risk of acquiring the disease but has not yet been diagnosed as having it; (b) inhibiting the disease, i.e., arresting its development; and/or (c) relieving the disease, i.e., causing regression of the disease. A therapeutic benefit includes eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In some aspects, for prophylactic benefit, treatment or compositions for treatment are administered to a subject at risk of developing a particular disease, or to a subject reporting one or more of the physiological symptoms of a disease, even though a diagnosis of this disease may not have been made. The methods of the present disclosure may be used with any mammal or other animal. In some aspects, treatment results in a decrease or cessation of symptoms. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the term “subject” refers to any individual or patient on which the methods disclosed herein are performed. The term “subject” can be used interchangeably with the term “individual” or “patient.” The subject can be a human, although the subject may be an animal, as will be appreciated by those in the art. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

As used herein, the term “effective amount” or “therapeutically effective amount” refers to that amount of an antibody, an antigen binding fragment thereof, or other composition described herein that is sufficient to induce the intended effect, including but not limited to disease treatment, as defined herein. The therapeutically effective amount may vary depending upon the intended treatment application (e.g., in vivo), or the patient and disease condition being treated, e.g., the weight and age of the patient, the severity of the disease condition, the manner of administration and the like, which can readily be determined by one of ordinary skill in the art. The term also applies to a dose that will induce a particular response in a target cell. The specific dose will vary depending on the particular antibody, an antigen binding fragment thereof, or other composition chosen, the dosing regimen to be followed, whether it is administered in combination with other compounds, timing of administration, the tissue to which it is administered, and the physical delivery system in which it is carried.

In some aspects, antibodies of the invention or antigen binding fragments thereof are used as a monotherapy or combined with other therapeutic agents such as radiotherapy, cytotoxic chemotherapy, immunotherapy including immune checkpoint inhibitors, and other immunoregulatory agents, such as vaccines, interleukins, cytokines, chemokines, and biologics as a combination therapy. Exemplary interleukins for immune therapy include IL-1, IL-2, IL-3, IL-4, IL-5, IL-6, IL-7, IL-8, IL-10, IL-12, IL-15, IL-18, IL-21, and IL-23. Exemplary cytokines for immune therapy include interferons, TNF-α, TGF-β, G-CSF, and GM-CSF. Exemplary chemokines for immune therapy include CCL3, CCL26, and CXCL7. Exemplary biologics include CAR T-cell therapy, tumor-infiltrating lymphocyte (TIL) therapy, and monoclonal antibodies, such as alemtuzumab, trastuzumab, ibritumomab tiuxetan, brentuximab vedotin, ado-trastuzumab emtansine, blinatumomab, bevacizumab, and cetuximab. Antibodies also include checkpoint inhibitors, including PD-1 inhibitors, such as pembrolizumab, nivolumab, and cemiplimab, for example, PD-L1 inhibitors, such as atezolizumab, avelumab and durvalumab, for example, CTLA-4 inhibitors, such as iplimumab, for example, and other checkpoint inhibitors, such as an anti B7-H3 antibody, an anti-KIR antibody and an anti-LAG3 antibody, for example.

In some embodiments, methods of treating cancer in a subject are provided herein. Methods of treating cancer include administering to a subject an amount of any bispecific antibody or an antigen binding fragment thereof provided herein, effective for treating the cancer.

Cancer is a group of diseases involving abnormal cell growth with the potential to invade or spread to other parts of the body. In 2015, about 90.5 million people had cancer, about 14.1 million new cases occur a year and it caused about 8.8 million deaths (15.7% of deaths). The most common types of cancer in males are lung cancer, prostate cancer, colorectal cancer and stomach cancer. In females, the most common types are breast cancer, colorectal cancer, lung cancer and cervical cancer. The term “cancer” refers to a group of diseases characterized by abnormal and uncontrolled cell proliferation starting at one site (primary site) with the potential to invade and to spread to others sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof. As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, rectal, liver, kidney, lung, testicular, breast, ovarian, pancreatic, brain, and head and neck cancers, melanoma, sarcoma, multiple myeloma, leukemia, lymphoma, and the like.

In some aspects, the cancer is prostate cancer, lung cancer, non-small cell lung cancer (NSCLC), melanoma, lymphoma, breast cancer, head and neck cancer, renal cell carcinoma (RCC), ovarian cancer, kidney cancer, urinary bladder cancer, uterine cancer, cervical cancer, ovarian cancer, liver cancer, stomach cancer, colon cancer, rectal cancer, oral cavity cancer, pharynx cancer, pancreatic cancer, thyroid cancer, skin cancer, brain cancer, bone cancer, hematopoietic cancer, or leukemia.

### Amino Acid and Nucleic Acid Sequences

In some embodiments, provided herein are isolated amino acid sequences encoding any of the antibodies, antigen binding fragment thereof or bispecific antibodies described herein.

The terms “peptide”, “polypeptide” and “protein” are used interchangeably herein and refer to any chain of at least two amino acids, linked by a covalent chemical bound. As used herein polypeptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

In various aspects, the amino acid sequences are as set forth in SEQ ID NOs: 1-40 and 46-91.

Also provided herein are isolated nucleic acid sequences encoding the amino acid sequences provided herein.

As used herein, the term “nucleic acid” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, or (iv) was synthesized, for example, by chemical synthesis. A nucleic can be employed for introduction into, i.e., transfection of, cells, in particular, in the form of RNA which can be prepared by in vitro transcription from a DNA template. The RNA can moreover be modified before application by stabilizing sequences, capping, and polyadenylation.

In some embodiments, provided herein are isolated nucleic acid sequences encoding any one of the amino acid sequences of SEQ ID NOs: 1-40 and 46-91.

In some embodiments, the invention further provides the expression, purification and characterization of anti-p95HER2 and anti-CD73 antibodies, as detailed in the examples below. A signal sequence can be included in expression constructs for antibodies provided herein. Any suitable signal sequence can be used, such as a sequence of SEQ ID NO:43.

Presented below are examples discussing anti-p95HER2 and anti-CD73 antibodies, antigen binding fragment thereof and bispecific antibody thereof, contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

Antibody Generation from the OmniMab Library

To generate antibodies against p95HER2, panning was carried out with the OmniMab phagemid library built on IgG variable region sequences collected from over a hundred healthy donors' PBMCs by AP Biosciences Inc. (AP Biosciences, Inc.). Phage display was carried out using Hyperphage (M13K07ΔρIII, Progen, Heidelberg, Germany).

Panning was performed using p95HER2-overexpressing HEK293 cells in the first round, then using recombinant p95HER2 protein produced by Expi-293 cells in the following three rounds selection. Briefly, for first round cell panning, the omniMab phage library was mixed with 1-fold 1% BSA in PBS. The phage/PBS mixture were added to the tube that contented wild type Expi293 cells for subtraction. Then wild type Expi293 cells were collected with centrifugation and washed cells three times with 0.5% BSA in PBS to collect the phage that didn't recognize Expi293 cells. And mixed the collection phage with full length p95HER2 transfected Expi293 with shaking. Washed transfected Expi293 cells with 0.5% BSA in PBS 10 times and eluted the phage that binding to p95HER2 transfected Expi293 cells with TEA solution. The eluted phages were collected to infect the TG1 for phage rescue.

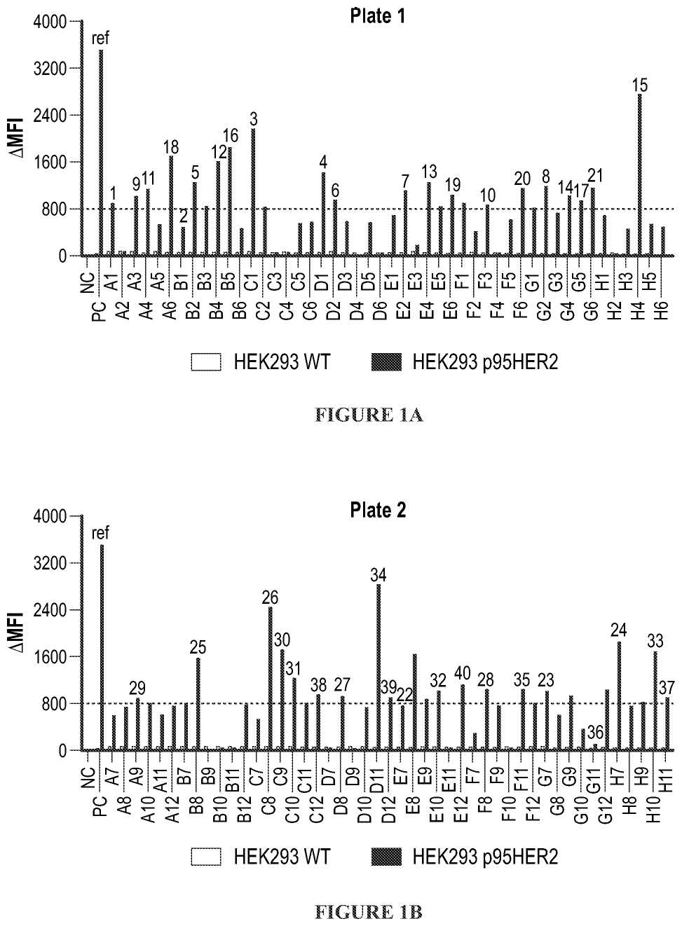

For second to fourth round solid phase panning, secretory p95HER2 ECD (ERBB2_HUMAN amino acid positions 611 to 652) with signal peptides and FLAG-taq was produced on Expi293 platform. The culture supernatant was collected with centrifugation as target antigen for panning using. Immune tubes were pre-coated with anti-FLAG mouse antibody with PBS and loaded with various FLAG-taq recombinant protein. The immune tubes and phage from previous panning rounds were mixed with 5% NFM or 1% BSA in PBS for blocking, then adding phage mixture to the preloading immune tubes containing FLAG-tag recombinant protein (except p95HER2 ECD-FLAG) for subtraction. Other immune tubes were added to the culture supernatant containing secretory p95HER2 ECD FLAG-tag. After incubation, collected and washed tubes with 1% tween-20 in PBS 3 times to collect the phage that didn't recognize the immune tubes. Then transfer the phage mixture to the immune tube containing p95HER2 ECD with FLAG-taq for antigen binding. After incubation, washed the immune tubes with 1% tween-20 in PBS 10 times and eluted the binding phage with TEA solution. The eluted phage were collected to infect the TG1 for phage rescue. And then, the single clones specific for p95HER2 were isolated and determined by FACS (FIGS. 1A-1C), and selected for sequencing to confirm the diversity of heavy chains and light chains. The reference antibody using in this study was generated from US2018118849A1 SEQ ID NOs: 20 and 21 with human IgG1 backbone and produced on ExpiCHO platform.

The unique antibody sequences were then sub-cloned and expressed for further analysis.

### Example 2

### Subcloning, Expression, and Purification of P95HER2 Specific Binding Proteins in the Form of IgGs

To quickly screen candidates, the heavy chains and light chains of positive p95HER2 binders identified by FACS were amplified, digested and subcloned into an in-house generated IgG expression vector carrying the IgG1 constant region (SEQ ID NO:41). After sequence validation, plasmids were prepared and transfected into ExpiCHO cells (Invitrogen) for antibody production. After 6 days of culture, antibody was affinity purified from culture supernatant by Protein A chromatography. Purified antibody was concentrated and dialyzed in PBS buffer. Antibody concentration was determined using NanoDrop2000 spectrophotometer. Antibody purity and integrity were determined by NuPAGE (ThermoFisher, Cat. No. NP0321BOX) under non-reducing or reducing conditions and visualized by Coomassic Brilliant Blue G-250 staining solution.

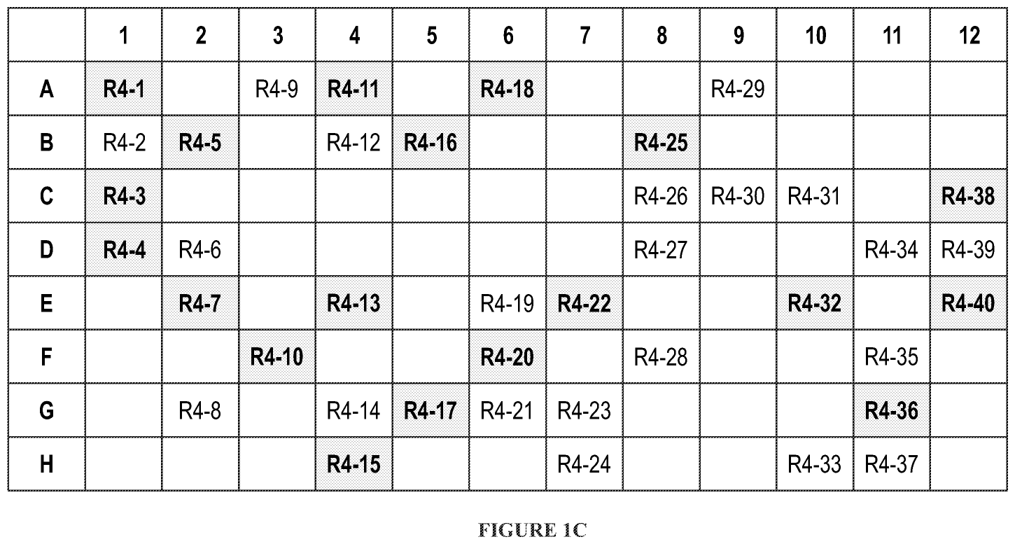

FIG. 2 shows the PAGE results of purified anti-p95HER2 antibody leads. Results indicated that proteins have a molecular weight of about 145 kDa under non-reducing conditions, and heavy chain and light chain have a molecular weight of 55 kDa and 25 kDa, respectively, under reducing conditions. Most of antibody leads showed greater than 90% purity from one-step Protein A chromatography.

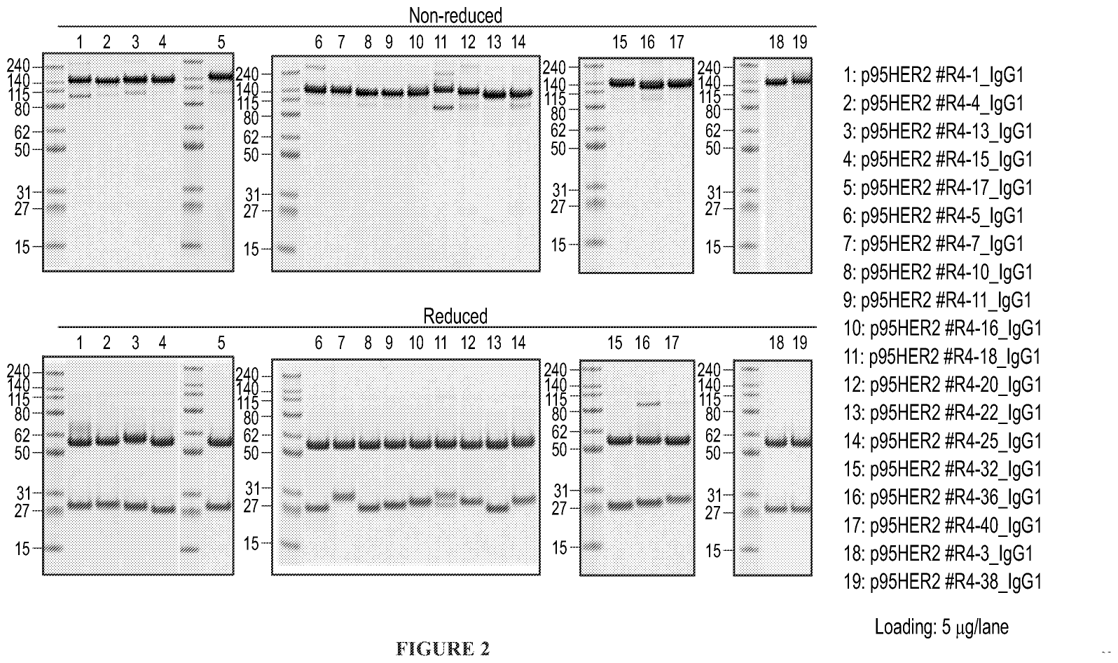

FIGS. 3A-3B show the binding activity of purified anti-p95HER2 antibody leads by flow cytometer.

### Example 3

### Binding Epitope Mappings of Anti-p95HER2 Monoclonal Antibody

The binding epitope mappings were conducted by PEPperPRINT Inc. To determine the binding epitopes, GS linker elongated full-length p95HER2 peptide sequence was converted into linear 5, 10 and 15 amino acid peptides each overlapped with 4, 9 and 14 amino acids.

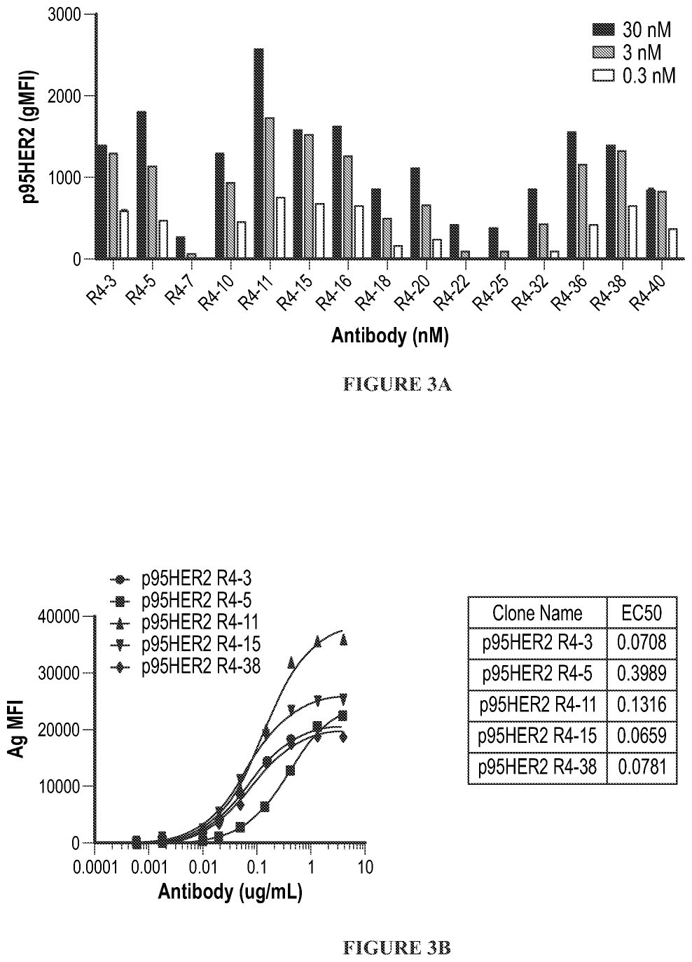

The linear peptide microarrays were incubated with the antibody leads at a concentration of 1 μg/ml followed by staining with secondary (Goat anti-human IgG (H+L) DyLight680) and control (Mouse monoclonal anti-HA (12CA5) DyLight800) antibodies as well as read-out with Odyssey Imaging System (LI-COR, Inc). Quantification of spot intensities and peptide annotation were done with PepSlide® Analyzer (SICASYS Software GmbH). The epitope for p95HER2 antibodies were shown and summarized in the FIG. 4A. The heat map of epitope mapping result showed that both anti-p95HER2 #R4-3, R4-5, R4-11, R4-15 and R4-38 could recognize motif KFPDE (SEQ ID NO:96) strongly. However, the signal from 10 and 15 amino acid peptides containing KFPDE motif disclosed that the structure of p95HER2 were not simple linear. Relatively stronger signal was generated when the motif KFPDE is located at the edge of the amino acid sequence, i.e., amino acid nearby motif KFPDE would affect the binding affinity of anti-p95HER2 antibodies (FIG. 4B).

### Example 4

### Evaluation of Protein Aggregation of Anti-p95HER2-CD137 Scfv Bispecific Antibody by Sec-HPLC

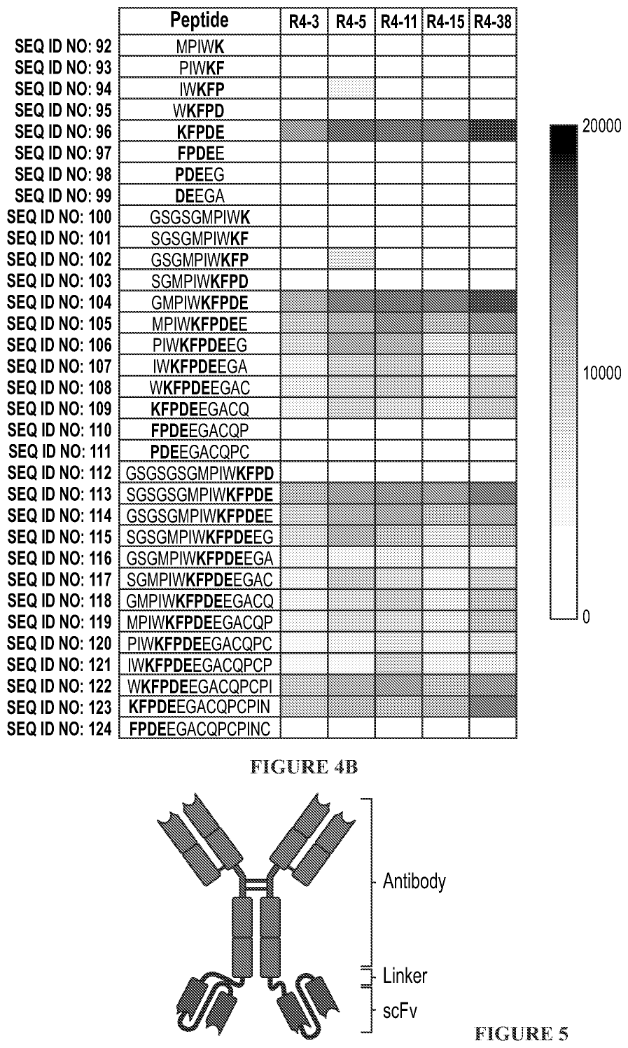

Purified and concentrated antibody (>1 mg/ml) was applied to determine variants and aggregations by SEC-HPLC analysis. SEC-HPLC was performed using a Waters ACQUITY Arc system with Waters 2489 UV/Vis detector. Samples were load into XBridge Protein BEH SEC Column (Waters, Cat #186007640) with isocratic 25 mM sodium phosphate, 200 mM NaCl, pH 6.8 as mobile phase buffer for SEC separation. The flow rate was 0.4 mL/min and the sample injection amount was 40 μg. Peaks were detected by absorbance at 280 nm. Before injection onto the SEC column, all samples were filtrated with 0.22 μm filter (Millipore, Cat #SLGP003RB) to remove any precipitated protein material. Data were analyzed by Empower 3 software. In summary, the purity of one-column Protein-A purified anti-p95HER2-CD137 antibodies are greater than 95% (FIG. 6A).

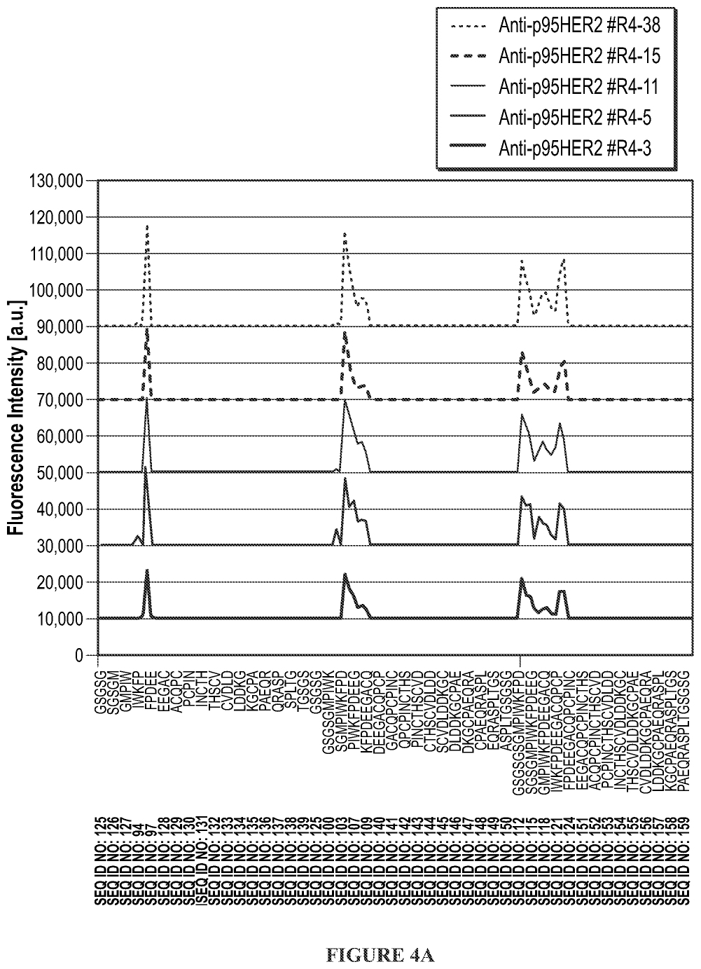

The general structure of the bispecific antibodies is presented in FIG. 5.

### Example 5

### Evaluation of Protein Integrity of Anti-p95HER2-CD137 Bispecific Antibodies by MCE-SDS-PAGE

All size variants analyses of purified antibody were performed on a LabChip GXII instrument (Perkin Elmer, Inc.). The assay was carried out with “Protein Clear HR Assay” as manufacture's protocol. Briefly, 2.5 μl of protein sample at 1 mg/mL was mixed with 18 μl of sample buffer. The sample buffer was prepared by mixing 700 μl of Protein Clear HR sample buffer with either 24.5 ul of 1M DTT (for the reducing assay) or 24.5 ul of 0.25 M NEM (for the non-reducing assay). The samples were incubated at 70° C. for 10 min. After cooling to room temperature, 35 μl of water was added to each sample before loading onto the instrument. The samples were then analyzed using the Protein Clear HR Assay script. Meanwhile, the chip was prepared as instructed by the manufacturer and was maintained at 30° C. throughout the analysis. The excitation and detection wavelengths are 630 nm and 700 nm, respectively. In summary, the purity and integrity of one-column Protein-A purified anti-p95HER2-CD137 antibodies reach about 95% (FIG. 6B).

### Example 6

### Binding Affinity of Anti-p95HER2-CD137 Bispecific Antibody by Biolayer Interferometry

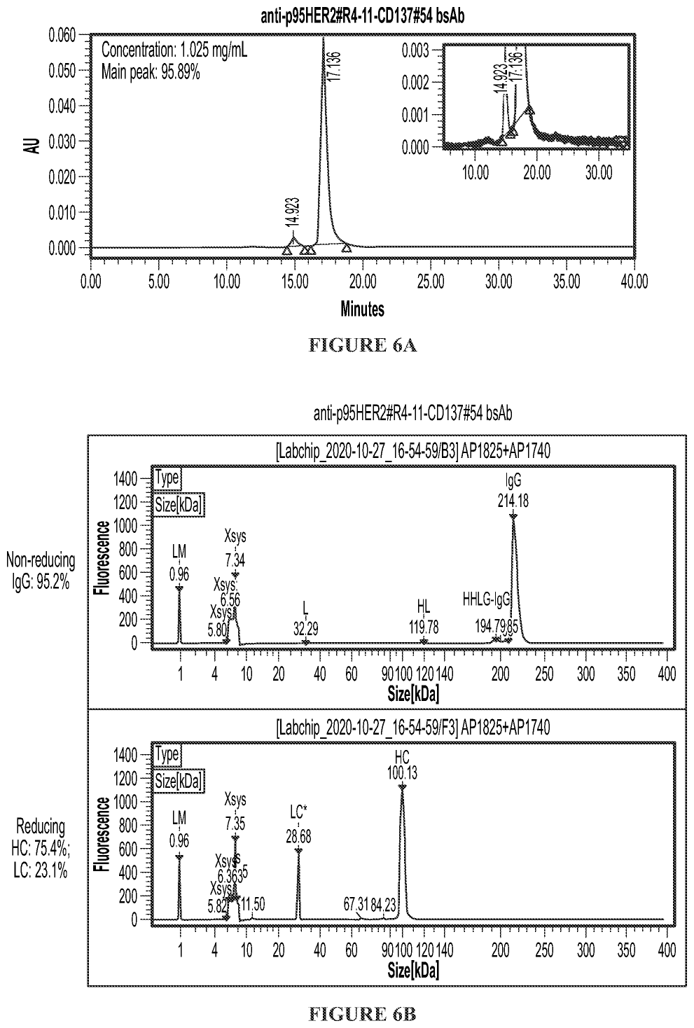

The affinity of anti-p95HER2-CD137 bsAbs were determined by biolayer interferometry analysis (ForteBIO, USA). To evaluate the binding affinity of anti-p95HER2-CD137 bsAbs against CD137, the bsAbs were loaded at 5 μg/mL on AHC (Anti-Human IgG Fc Capture, ForteBIO, Cat. No. 18-5060) for signal reach to approximate 0.5 nm. Sensors then were exposed to 7 series of concentration analytes which was CD137 (Cat #41B-H5256) extracellular domain fused with mouse Fc tag recombinant protein. To determine the affinity of anti-p95HER2-CD137 bsAbs against p95HER2, custom synthesis biotinylated Her2 p95 peptide (GenScript) was loaded at 5 ng/ml on SA (Streptavidin, ForteBIO, Cat #18-5019) biosensor for 1 min. Sensors then were exposed to 7 series of concentration analytes which was bsAbs. Measurements were performed as follows: 1 min baseline in kinetic buffer, 1 min baseline in kinetic buffer, 5 minutes association in 7 different concentration analytes and 5 minutes dissociation into kinetic buffer. The kinetic parameters were calculated and produced by Octet Data Acquisition and Analysis Software. In summary, the affinity (K_{D}) of anti-p95HER2 #R4-11-CD137 #54 bsAb against p95HER2 and CD137 antigen was 0.189 and 1.12 nM, respectively (see FIGS. 7A-7B).

### Example 7

The Simultaneous Binding to p95HER2 and CD137 by Anti-p95HER2 #R4-11-CD137 #54 bsAb

The binding activities of anti-p95HER2-CD137 bispecific antibody was determined by ForteBIO® (Menlo Park, Calif.) biosensor analysis. In this method, the custom synthesis biotinylated Her2 p95 peptide (GenScript) was loaded onto SA (Streptavidin, ForteBIO, Cat #18-5019) biosensor at 50 μg/mL for 5 minutes. Sensors then were exposed to antibody samples at 100 nM for 5 minutes follow by associating with the mouse Fc tagged human CD137 protein (AcroBiosystems, Cat #41B-H5256) at 100 nM for 5 minutes. The binding chart were produced by Octet Data Acquisition and Analysis Software. In summary, these results show that anti-p95HER2-CD137 bispecific antibodies simultaneously recognize p95HER2 and CD137, as determined by ForteBIO® biosensor analysis (FIG. 8).

### Example 8

p95HER2-Dependent T Cell Activation Induced by Anti-p95HER2-CD137 Bispecific Antibody

Human T cells were isolated using a RosetteSep™ Human T Cell Enrichment Cocktail (STEMCELL Cat. No. 15061). Purified T cells (1×10⁵ cells) were cocultured with p95HER2-expressing NCI-H292 lung cancer cells (1×10⁴ cells) in the presence of anti-CD3 (OKT3)-coated polybeads at T-cell:bead ratio 1:1. Three days later, T cell activation was measured by IFN-γ production as measured by ELISA.

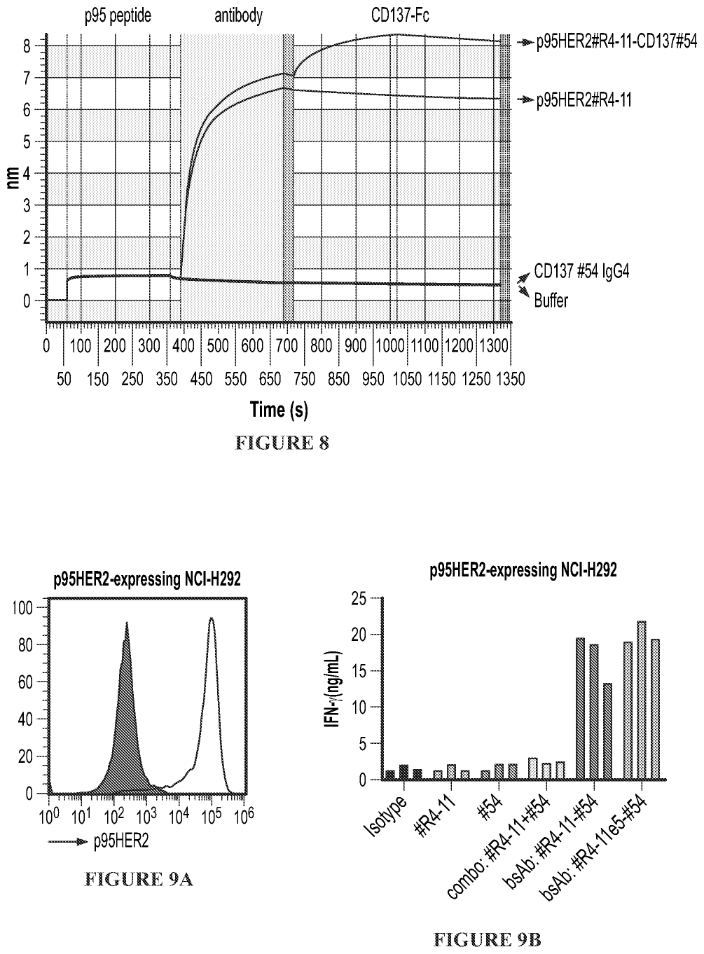

The expression level of p95HER2 on NCI-H292 lung cancer cells were checked by flow cytometry (FIG. 9A). More robust IFN-γ production was induced by anti-p95HER2-CD137 bsAb compared to the mono- or combination therapy with anti-p95HER2 and anti-CD137 #54 antibodies (FIG. 9B).

### Example 9

### Antibody-Dependent Cell-Mediated Cytotoxicity (ADCC) by Anti-p95HER2-CD137 Bispecific Antibody

For ADCC induction, CD16-NK92 effector cells were cocultured with CD137-expressing HEK293 target cells (2×10⁴ cells) at E/T ratio 1, 3 and 10 in a well of U-bottom 96-well plate. Two anti-p95HER2-CD137 bsAbs, #R4-11-#54 (with original IgG1 Fc) and #R4-11e5-#54 (with ADCC-enhancing IgG1 Fc, SEQ. NO. 42), were applied in this study. Six hours later, cytotoxicity activity was determined by CytoTox 96® Non-Radioactive Cytotoxicity Assay (Promega, G1780).

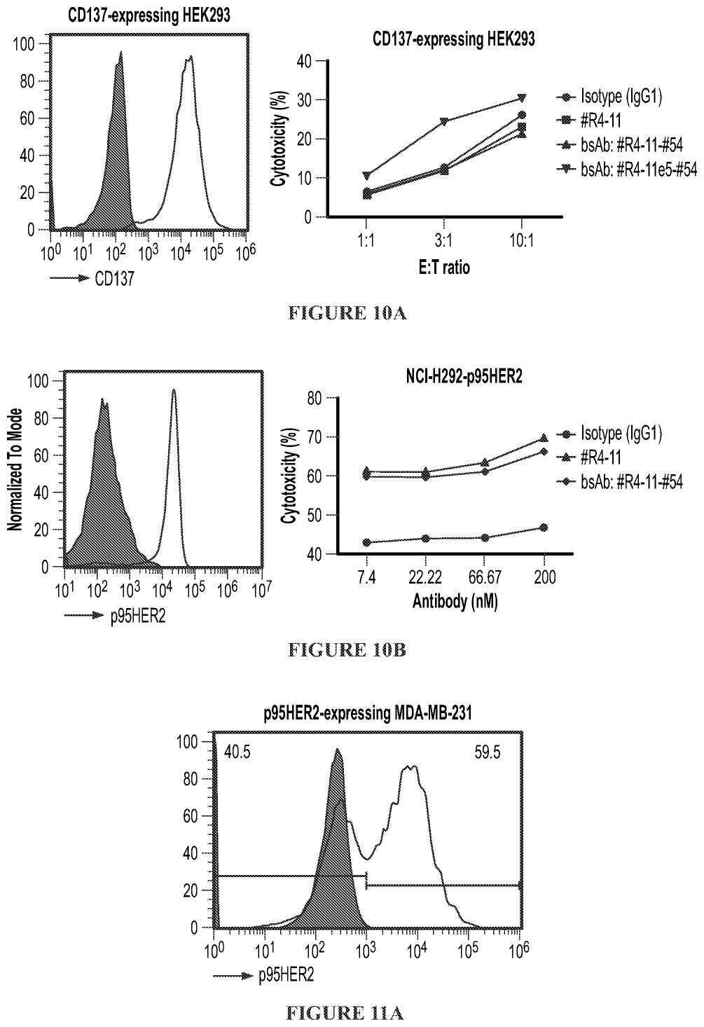

Compared to isotype control, #R4-11c5-#54, but not #R4-11 and #R4-11-#54, induced the ADCC in CD137-HEK293 cells. This was corresponding to the ADCC-enhancing mutations in the IgG1 Fc region of #R4-11e5-#54 (FIG. 10A). #R4-11-#54 induced the ADCC activity against p95HER2-NCI-H292 cells as well as its parental #R4-11 mAb (FIG. 10B).

In summary, these results indicated that #R4-11c5-#54 bsAb could be deleterious to CD137-expressing cells, like NK and T cells, which are critical effector cells in cancer. Therefore, #R4-11-#54 bsAb with normal IgG1 Fc was selected for further development.

### Example 10

### Inhibition of Tumor Growth by Anti-p95HER2-CD137 Bispecific Antibody In Vivo.

To validate the anti-tumor activity of anti-p95HER2 #4-11-CD137 #54 bsAb, which does not cross-react with mouse CD137, human tumor cells (p95HER2-expressing MDA-MB-231) were premixed with human PBMC and xenografted orthotopically to forth mammary fat pad of female SCID-beige mice to evaluate anti-cancer activity in vivo. Ten days post tumor inoculation, equal moles of mAb (M.W. 150 kDa, 7.7 mg/kg) and bsAb (M.W. 195 kDa, 10, 2, 0.4 mg/kg) were intraperitoneally injected twice per week. Tumor sizes (mm3) were measured twice per week and calculated as (length×width×width)/2. Tumor inhibition ratio was calculated by the following equations based on the tumor volume (TGIv) or tumor weight (TGIw). TGIv (%)=[1−(Vₜ−V₀ of treatment group)/(Vₜ−V₀ of control group)]×100%, which Vₜ: mean tumor volume after treatment; V₀: mean tumor volume before treatment. TGIw (%)=(mean tumor weight of control group−mean tumor weight of treatment group)/mean tumor weight of control group×100%.

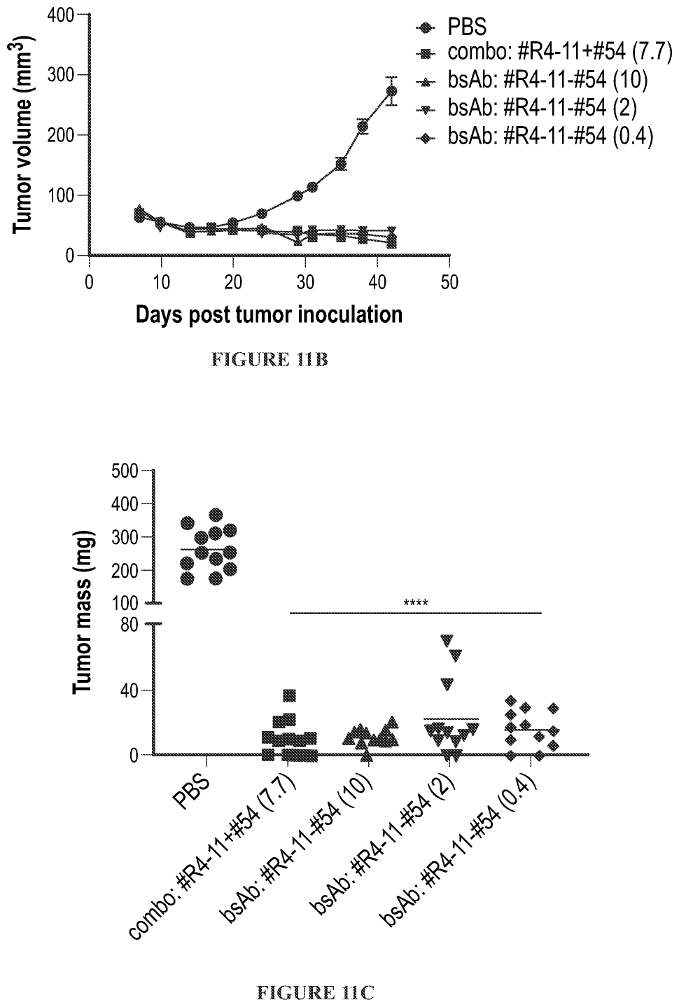

In summary, these results show significant inhibition of tumor growth upon treatment with compared to the treatment with PBS (FIGS. 11A-11C). The greater tumor inhibition was shown by TGI_{w} as 95.6%, 91.5% and 93.7%, while TGIᵥ was 125.5%, 113% and 117%, at 10, 2 and 0.4 mg/kg, respectively.

### Example 11

### Pharmacokinetic Profile of Anti-p95HER2-CD137 Bispecific Antibody in Mice

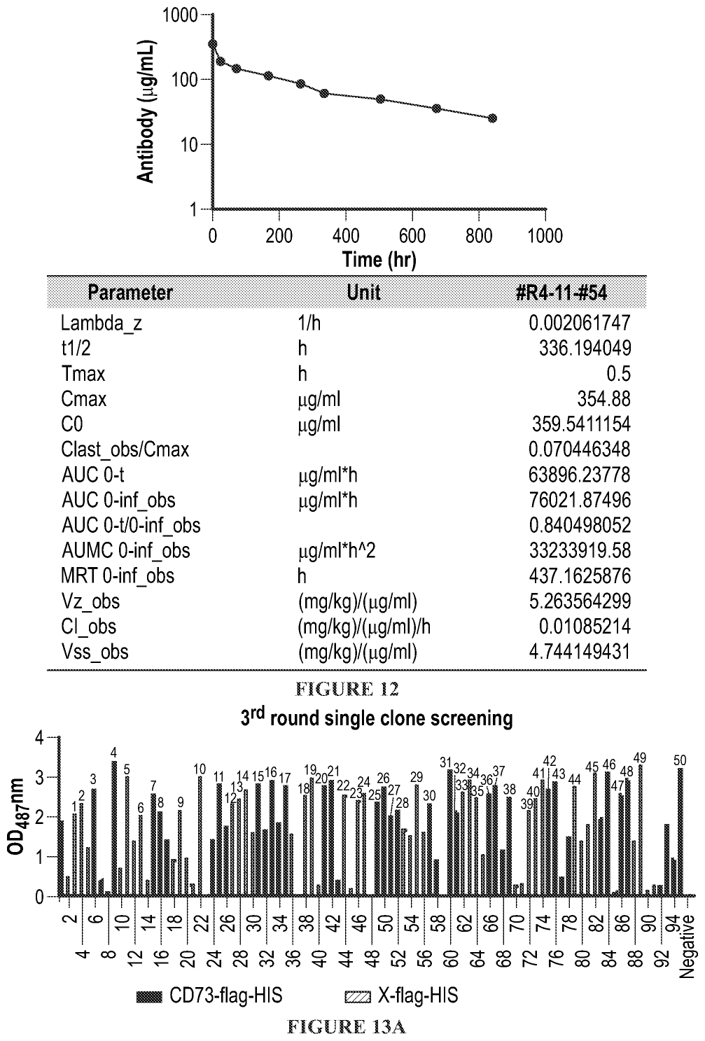

The bispecific anti-p95HER2-CD137 antibody (5 mg per kg body weight) was administered via an intravenous bolus injection to SCI-beige mice. Peripheral blood was collected at 0.5, 24, 72, 168, 264, 336, 504, 672 and 840 hours post injection. Plasma concentrations of the antibody were determined by ELISA. MaxiSorp plates (Invitrogen) were coated with CD137-Fc fusion protein (AP Biosciences, 0.5 μg/mL), followed by application of serially diluted plasma samples and anti-CD73-CD137 #54 bsAb as the standard curve. Bound antibodies were detected by CD73-Flag-His antigen (AP Biosciences) and HRP-conjugated anti-Flag antibody using TMB substrate. Plasma antibody concentrations were calculated by the interpolation method. PK parameters were calculated using PKSolver software. Anti-p95HER2-CD137 showed PK profile with tin 336 hours and AUC 63896 μg/ml*h (FIG. 12).

### Example 12

Antibody Generation from the OmniMab Library

To generate antibodies against CD73, selections with an OmniMab phagemid library were carried out. The phagemid library was built up by AP Biosciences Inc. (APBio Inc.) from a collection of peripheral blood mononuclear cells from over a hundred healthy donors.

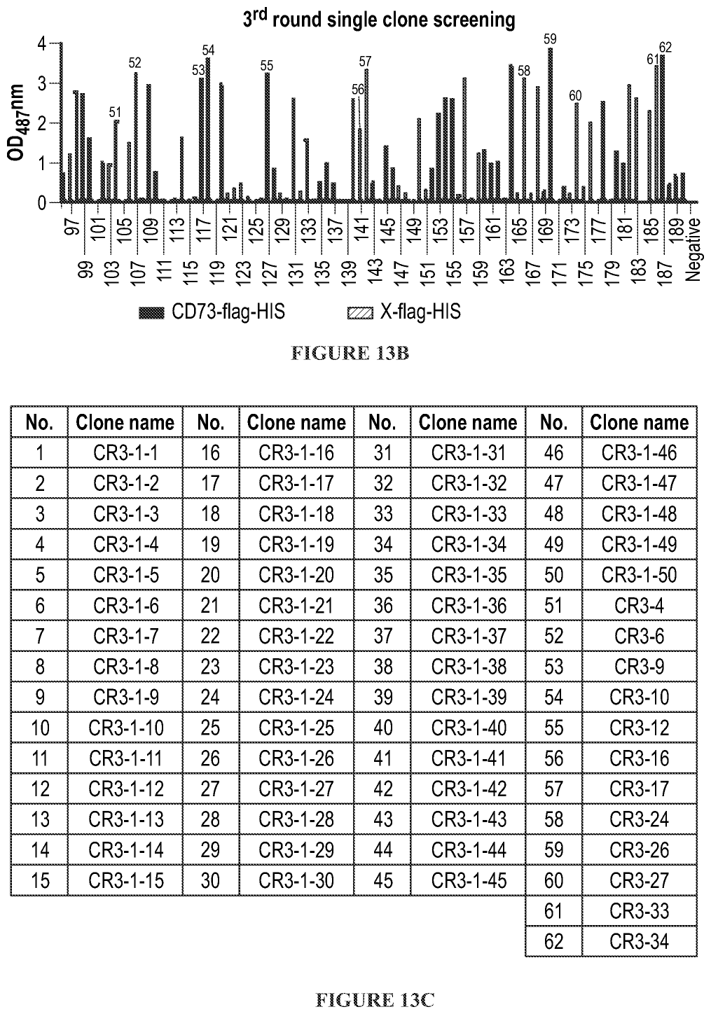

First round panning was performed using Hyperphage (M13K07ΔpIII, Progen, Heidelberg, Germany). Solution phase panning was used for CD73 specific binders' selection and isolation from the OmniMab library. Solution phase panning was performed using recombinant CD73 protein carries flag tag and polyhistidine tag at the C-terminus which produced by mammalian cell expression system used in first-round selection. Second round enrichment was also used solution phase panning. CD73-expressing Expi293 cells and breast cancer cell line MDA-MB-231 were used for third round enrichment separately. The specific CD73 binders were screened and isolated by direct ELISA and FACS after three round panning (FIGS. 13A-13C). The phage clones recognize human CD73 specifically after three rounds human CD73 specific enrichment process from OmniMab phage library. Phage-containing supernatant were applied to CD73 ELISA, and positive binders were numbered. MDA-MB-231 breast cancer cell line was stained with CD73 phages supernatant (50 μl/well) which were isolated from MDA-MB-231 cell-based panning to examine CD73 binding activity. FIG. 13B showed ranking of binders binding activity. Binders were isolated and sent for sequencing to confirm the sequence and diversity of the heavy chain.

### Example 13

### Subcloning, Expression, and Purification of CD73-Specific Binding Proteins in the Form of IgGs

To quickly screen candidates, the heavy chains and light chains of positive CD73 binders identified by ELISA and FACS were amplified, digested and subcloned into an IgG expression vector generated by APBio and carrying the engineered IgG1 constant region (SEQ ID NO: 87). After sequence validation, plasmids were prepared and transfected into ExpiCHO cells (Invitrogen) for antibody production. After 6 days of culture, antibody secreted into serum-free medium was affinity purified from culture supernatant by Protein A chromatography. Purified antibody was concentrated, followed by dialysis in PBS buffer. The final concentration of dialyzed protein was determined using a NanoDrop2000 spectrophotometer and the purity and integrity were determined by SDS-PAGE with or without reducing reagent. Purified antibodies were analyzed by NuPAGE (ThermoFisher, Cat. No. NP0321BOX) under non-reducing or reducing conditions. Once the dye front has reached the bottom of the gel, turn off the voltage and remove the gel from the glass plates. Place the gel in Coomassie Brilliant Blue G-250 staining solution in a plastic container and rock for several hours. The destain can be poured off into the designated waste container in the fume hood and replaced several times until the bands can be clearly visualized against the background of the gel.

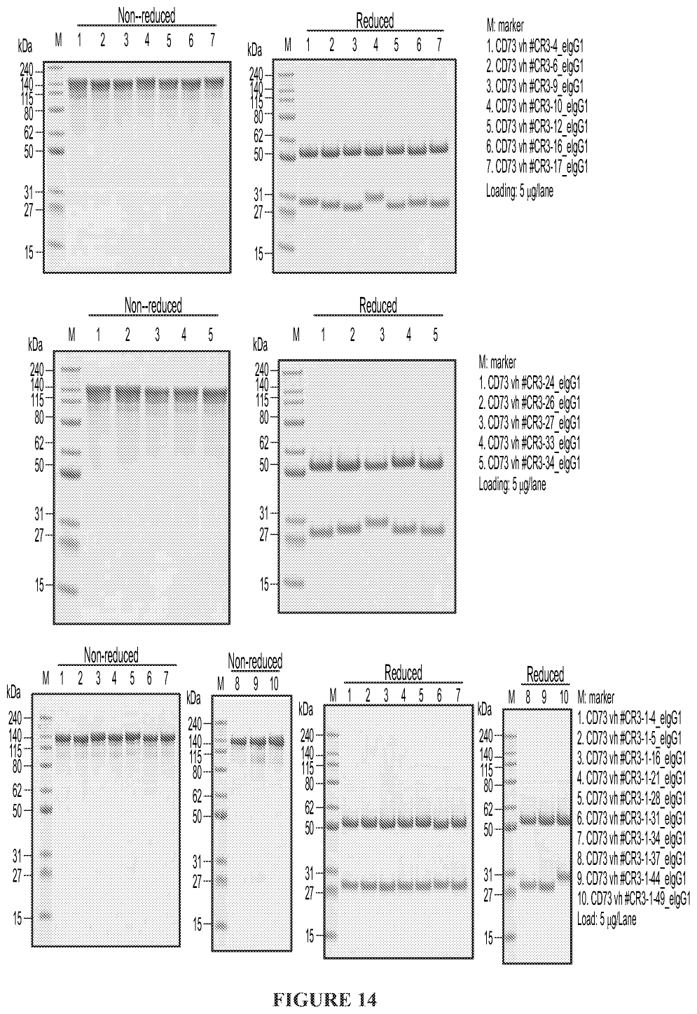

FIG. 14 shows representative PAGE gel analysis of different batches of purified anti-CD137 antibody leads. The purity and integrity of one-step Protein A-purified anti-CD73 antibody leads was analyzed by SDS-PAGE under non-reducing and reducing conditions. Results indicated that proteins have a molecular weight of about 145 kDa under non-reducing conditions, and heavy chain and light chain have a molecular weight of 55 kDa and 25 kDa, respectively, under reducing conditions. More than 90% purity could be obtained by one step of Protein A chromatography.

### Example 14

### Blockade of CD73 Enzymatic Activity by Anti-CD73 Antibody

MDA-MB-231 cells (2×10+ cells) were incubated with anti-CD73 antibody in a well of 96-well tissue-cultured plate at 4° C. for 30 minutes. AMP (12.5 μM, Sigma, Cat. No. A1752) was added and incubated at 4° C. for additional one hour. Supernatants were transferred to 96-well white plate, and residual AMP was detected by AMP-Glo assay followed manufacturer's instruction (Promega, Cat. No. V5011). The percentage of enzyme activity inhibition was calculated as [(Cells+AMP+antibody)−(Cells+AMP)]/[AMP−(Cells+AMP)]×100. To study the blockade activity of anti-CD73 antibody to soluble CD73, MDA-MB-231 cells (4.8×10⁶ cells) seeded in a well of 24-well plate for 40 hours, then cultured medium were collected and incubated with antibodies at 37° C. for 1 hour. AMP (12.5 μM, Sigma, Cat. No. A1752) was added and incubated at 37° C. for additional 2 hours, followed by the same procedures described above to detect residual AMP and calculate the percentage of enzyme inhibition.

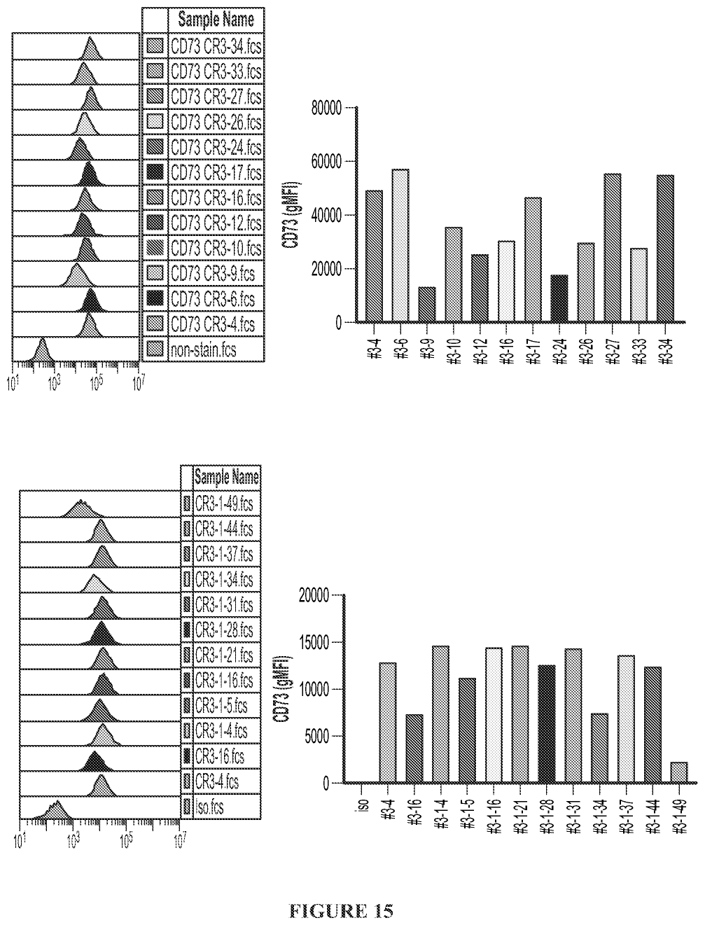

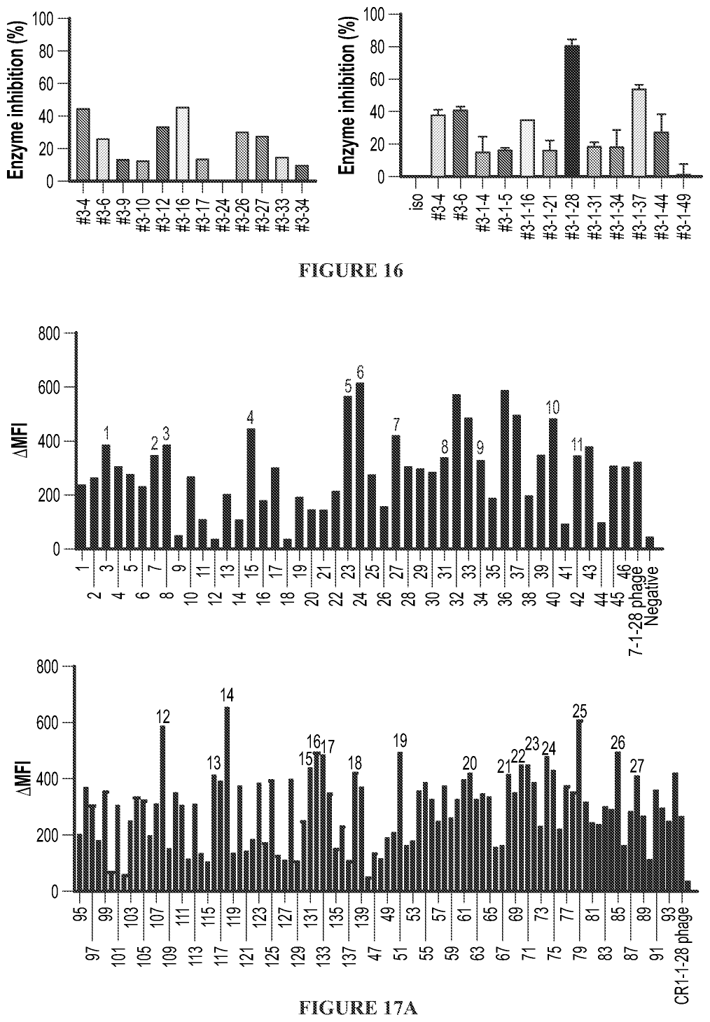

In summary, the binding affinity of anti-CD73 antibody did not necessarily correspond to their blockade of enzymatic activity. Although possess the better binding activity than clone #3-4, the weaker blockade of CD73 enzymatic activity was observed in clone #3-6, #3-27, #3-34, #3-1-14, #3-1-21, and #3-1-31. Interestingly, clone #3-1-28 showed the most potent blockade of CD73 enzymatic activity even with a comparable binding activity to clone #3-4, and #3-1-28 was selected for affinity maturation (FIG. 15 and FIG. 16). As illustrated in FIG. 15 the binding activity of anti-CD73 antibody leads was assessed by flow cytometer. MDA-MB-231 cells (1×105 cells) were surface stained with anti-CD73 antibody (4 μg/ml) for 30 minutes, then incubated with A488-conjugated anti-human IgG secondary antibody for 15 minutes on ice. Cells were analyzed by flow cytometer, and A488 fluorescence intensity was shown by histograms (left) and number (right table). As illustrated in FIG. 16 the blockade of CD73 enzymatic activity by anti-CD73 antibody leads was assessed. The enzymatic activity of CD73 expressed by MDA-MB-231 on the cell surface was blocked by anti-CD73 antibody leads.

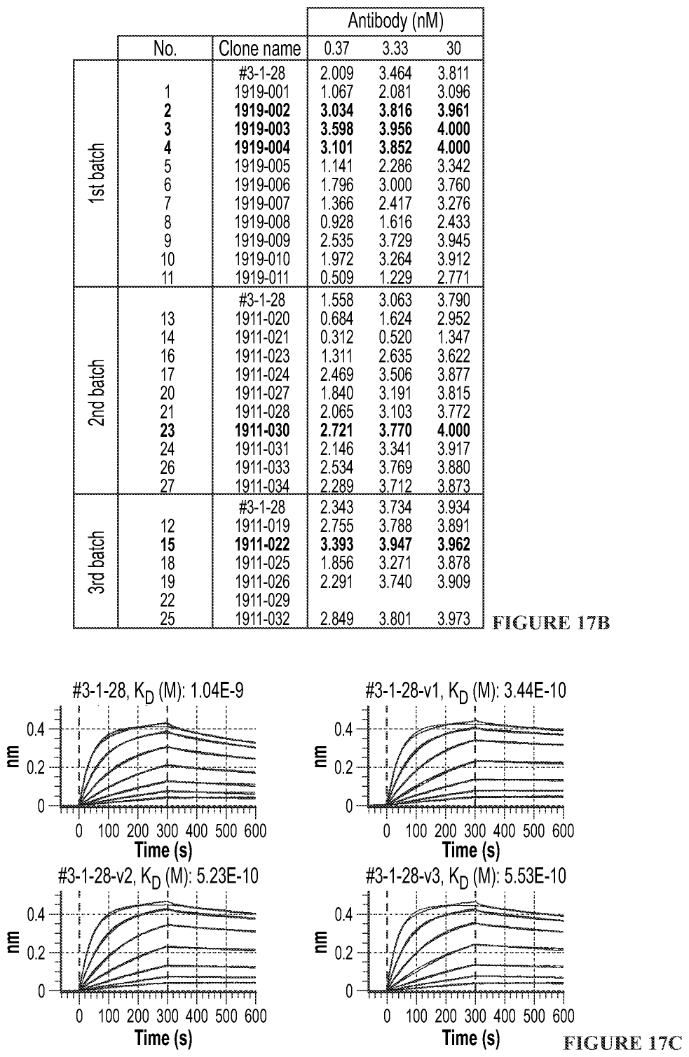

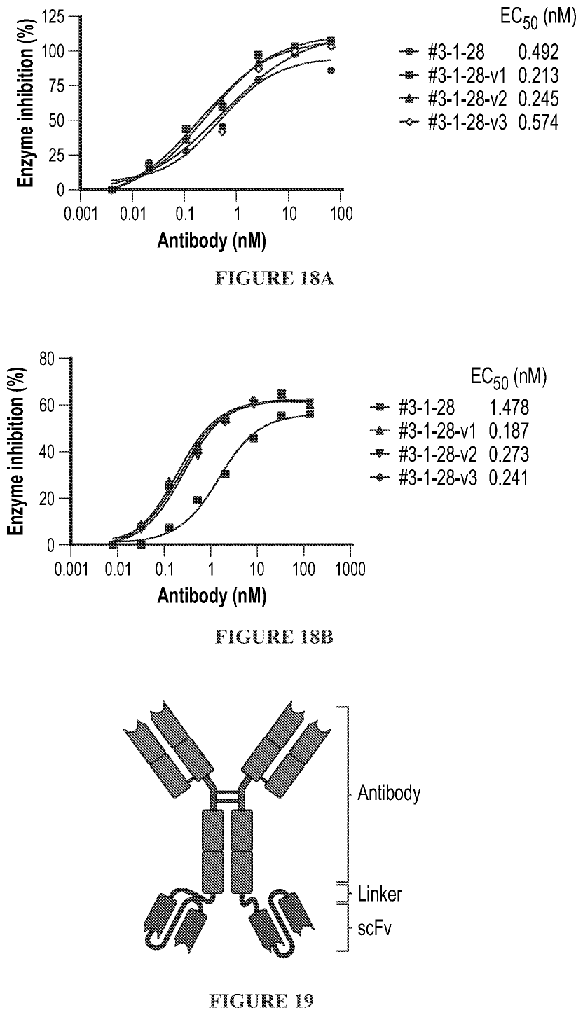

Corresponding to the improved binding affinity (FIGS. 17A-17C and EXAMPLE 15), the CD73 enzyme block activity of anti-CD73 clone #3-1-28-v1, #3-1-28-v2, and #3-1-28-v3 was better than their parental clone #3-1-28 (FIG. 18). In the inhibition of cell membrane-bound CD73 enzyme, the IC₅₀ of anti-CD73 #3-1-28-v1 (0.213 nM) and anti-CD73 #3-1-28-v2 (0.245 nM) was about 2 folds lower than that of parental #3-1-28 (0.49 nM) (FIG. 18A). Interestingly, in the inhibition of soluble CD73 enzyme, the IC₅₀ of anti-CD73 #3-1-28-v1 (0.187 nM), anti-CD73 #3-1-28-v2 (0.273 nM), and anti-CD73 #3-1-28-v3 (0.241 nM) was even more than 5 folds lower than that of parental #3-1-28 (1.478 nM) (FIG. 18B).

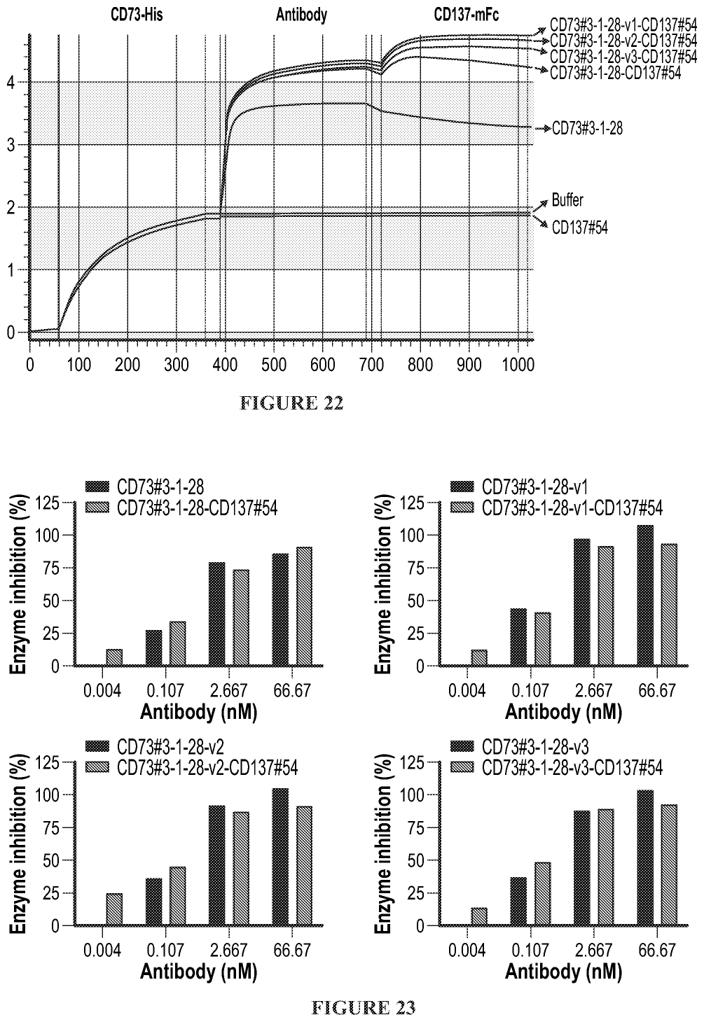

Furthermore, conversion of affinity-maturated anti-CD73 #3-1-28-v1, #3-1-28-v2, and #3-1-28-v3 to the bispecific antibody by linking CD137 scFv to their Fc C-terminal did not change their enzymatic blockade activities (FIG. 23).

### Example 15

### Affinity Maturation of Anti-CD73 #3-1-28

Affinity-matured antibodies can exhibit increased biological efficacy. Regardless of whether an antibody is isolated from a hybridoma or a human Fv phage library. An increased affinity may also allow for a reduced dosage of a therapeutic antibody and toxic side effects may be reduced. Germline hotspot residues are most likely to have a major impact on affinity based on statistical analysis of independent monoclonal antibodies derived from the same germline genes recognizing the same epitopes. Germline hotspots in the antibody complementarity determining regions (CDR) are naturally prone to hypermutations (Neuberger and Milstein, 1995). To improve #3-1-28 binding activity, we introduced random mutations into LCDR2/HCDR3 and made phage-display libraries with a diversity around 10\hat{ }2 and 10\hat{ }8 in these two CDR regions, respectively. Panning of these hotspot libraries has yielded mutant antibodies with increased affinity. CD73-expressing MDA-MB-231 breast cancer cells were used for three rounds cell-based panning enrichment. Binders with better binding activity than parental phage #3-1-28 were screened by FACS (FIG. 17A), and 27 binders were sequenced to confirm the diversity of the engineered CDR region. The heavy chains and light chains of positive CD73 binders identified by FACS were amplified, digested and subcloned into an IgG expression vector carrying the engineered IgG1 constant region. Vectors were transfected into the mammalian cell expression system for IgG production.

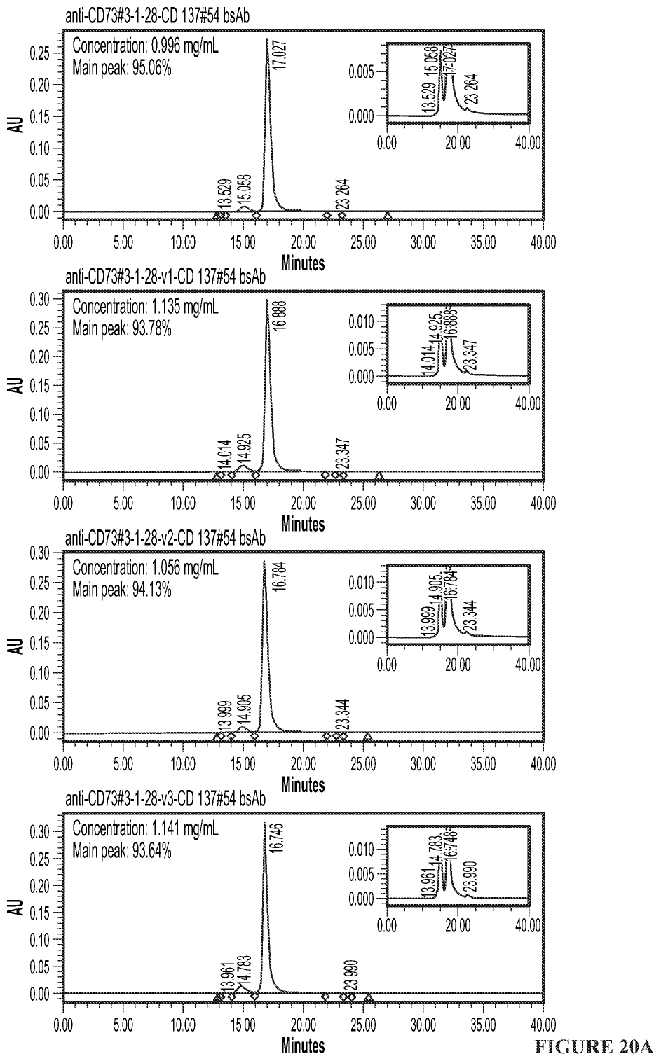

Based on the improved binding measured by ELISA and biolayer interferometry (FIGS. 17B and 17C), clone #1919-003, #1911-022, and #1911-030 were selected for further development of bispecific antibodies (having a general structure as illustrated in FIG. 19). To emphasize the derivation from parental #3-1-28, clone #1919-003, #1911-022, and #1911-030 were re-named hereafter as #3-1-28-v1, #3-1-28-v2, and #3-1-28-v3, respectively. As illustrated in FIG. 17A, CD73 binding activities of phage clones from small-scale affinity maturation library were determined by staining of CD73-expressing MDA-MB-231 cells and analyzed by flow cytometer. Clones with the better binding activity compared to parental phage clone #3-1-28 were numbered. As shown in FIG. 17B, the binding activities of antibodies converted from phage clones numbered in FIG. 17A were measured by CD73 ELISA. The clones in bold #1919-002, #1919-003, #1919-004, #1911-022, and #1911-030 showed the better binding activity than parental #3-1-28 antibody. As illustrated in FIG. 17C, binding kinetics of antibodies selected in FIG. 17B were measured by biolayer interferometry. To emphasize the derivation from clone #3-1-28, #1919-003, #1911-022, and #1911-030 were named hereafter as #3-1-28-v1, #3-1-28-v2, and #3-1-28-v3, respectively. Based on the detected Kp value, the affinities of #3-1-28-v1, #3-1-28-v2, and #Mar. 1, 2028-v3 were improved about 2 folds compared to parental #3-1-28.

### Example 16

### Binding Affinity of Anti-CD73 Monoclonal Antibody and Anti-CD73-CD137 Bispecific Antibody by Biolayer Interferometry

The affinity of anti-CD73 mAbs and bsAbs were determined by biolayer interferometry analysis (ForteBIO, USA). In this method, anti-CD73 antibodies were loaded at 5 μg/mL on AHC (Anti-Human IgG Fc Capture, ForteBIO, Cat. No. 18-5060) for signal reach to approximate 0.5 nm. Sensors then were exposed to his-tagged human CD73 (AcroBiosystems, Cat. No. CD3-H52H7)-containing solutions that 2-fold serial diluted from the top concentration 30 nM for total 7 points. Measurements were performed as follows: 1 minute baseline in kinetic buffer, 5 minutes association in hCD73-His-containing solutions, and 5 minutes dissociation into kinetic buffer. The kinetic parameters were calculated and produced by Octet Data Acquisition and Analysis Software.

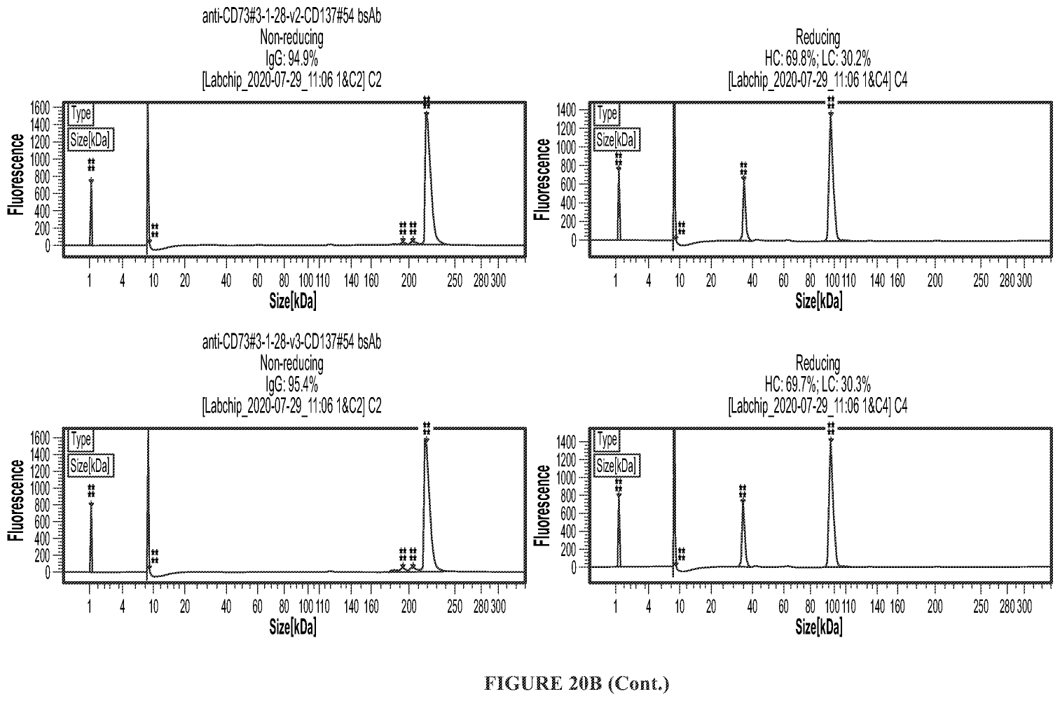

In summary, binding affinity of anti-CD73 #3-1-28-v1, #3-1-28-v2, and #3-1-28-v3 show about 2-fold improvement after affinity maturation compared to parental #3-1-28 (FIG. 17C). The improved CD73 binding affinities of #3-1-28-v1, #3-1-28-v2, and #Mar. 1, 2028-v3 remains after conversion to anti-CD73-CD137 bispecific antibodies (FIGS. 21A-21B).

### Example 17

### Evaluation of Protein Aggregation of Anti-CD73-CD137 SCFV Bispecific Antibody by SEC-HPLC

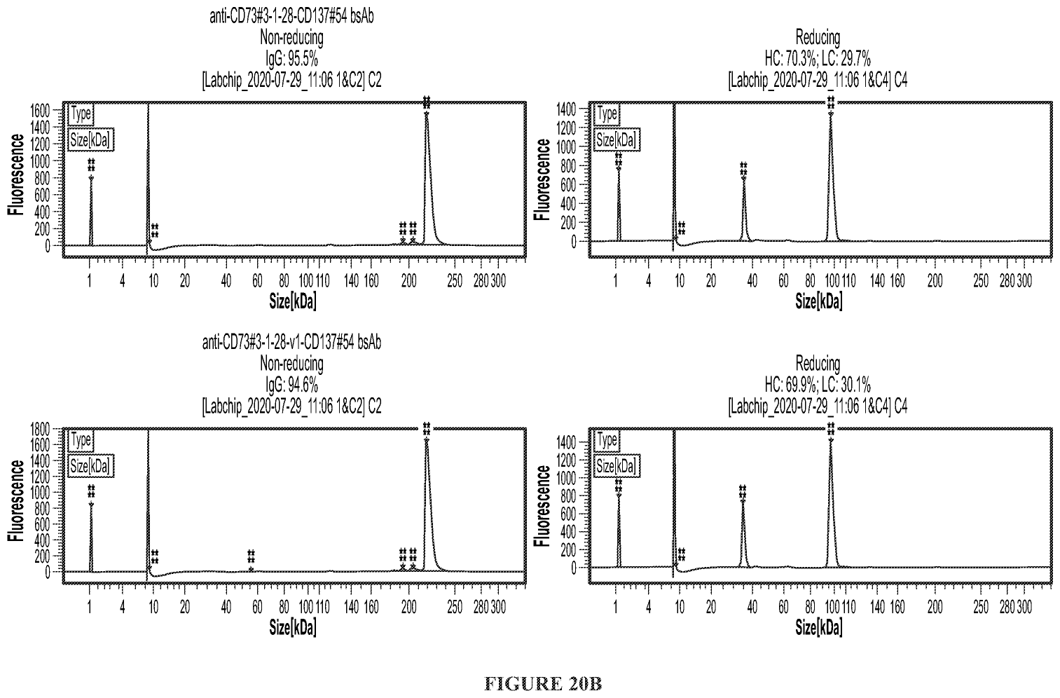

Purified and concentrated antibody (>1 mg/ml) was applied to determine variants and aggregations by SEC-HPLC analysis. SEC-HPLC was performed using a Waters ACQUITY Arc system with Waters 2489 UV/Vis detector. Samples were load into XBridge Protein BEH SEC Column (Waters, Cat #186007640) with isocratic 25 mM sodium phosphate, 200 mM NaCl, pH 6.8 as mobile phase buffer for SEC separation. The flow rate was 0.4 mL/min and the sample injection amount was 40 μg. Peaks were detected by absorbance at 280 nm. Before injection onto the SEC column, all samples were filtrated with 0.22 μm filter (Millipore, Cat #SLGP003RB) to remove any precipitated protein material. Data were analyzed by Empower 3 software. In summary, the purity of one-column Protein-A purified anti-CD73-CD137 antibodies are greater than 90% (FIGS. 20A-20B).

### Example 18

### Evaluation of Protein Integrity of Anti-CD73-CD137 Bispecific Antibodies by MCE-SDS-PAGE

All size variants analyses of purified antibody were performed on a LabChip GXII instrument (Perkin Elmer, Inc.). The assay was carried out with “Protein Clear HR Assay” as manufacture's protocol. Briefly, 2.5 μl of protein sample at 1 mg/mL was mixed with 18 μl of sample buffer. The sample buffer was prepared by mixing 700 μl of Protein Clear HR sample buffer with either 24.5 μl of 1M DTT (for the reducing assay) or 24.5 μl of 0.25 M NEM (for the non-reducing assay). The samples were incubated at 70° C. for 10 min. After cooling to room temperature, 35 μl of water was added to each sample before loading onto the instrument. The samples were then analyzed using the Protein Clear HR Assay script. Meanwhile, the chip was prepared as instructed by the manufacturer and was maintained at 30° C. throughout the analysis. The excitation and detection wavelengths are 630 nm and 700 nm, respectively. In summary, the purity and integrity of one-column Protein-A purified anti-CD73-CD137 antibodies reach about 95% (FIGS. 20A-20B).

### Example 19

### Antigen Recognition by Anti-CD73-CD137 Bispecific Antibodies

The binding activities of the AP601 was determined by ForteBIO® (Menlo Park, Calif.) biosensor analysis. In this method, the His tagged human CD73 protein (AcroBiosystems, Cat. No. CD3-H52H7) were loaded onto HIS1K (Anti-Penta-HIS) biosensor (Cat. No. 18-5120) at 5 μg/mL for 5 minutes. Sensors then were exposed to antibody samples at 100 nM for 5 minutes follow by associating with the mouse Fc tagged human CD137 protein (AcroBiosystems, Cat. No. 41B-H5256) at 100 nM for 5 minutes. The binding chart were produced by Octet Data Acquisition and Analysis Software.

In summary, these results show that anti-CD73-CD137 bispecific antibodies simultaneously recognize CD73 and CD137, as determined by ForteBIO® biosensor analysis (FIG. 22).

### Example 20

### Rescue of Amp-Suppressed T-Cell Proliferation by Anti-CD73-CD137 Bispecific Antibody

Human T cells were isolated using a RosetteSep™ Human T Cell Enrichment Cocktail (STEMCELL Cat. No. 15061). Purified T cells were labeled with CFSE (ThermoFisher, Cat. No. C34554) and stimulated with human T-Activator CD3/CD28 dynabeads (ThermoFisher, Cat. No. 11131D) at T-cell:bead ratio 1:0.5 in the presence of AMP (400 μM, Sigma, Cat. No. A1752). Three days later, T-cell activation was determined by IFN-γ production measured by ELISA and cell proliferation analyzed by flow cytometry.

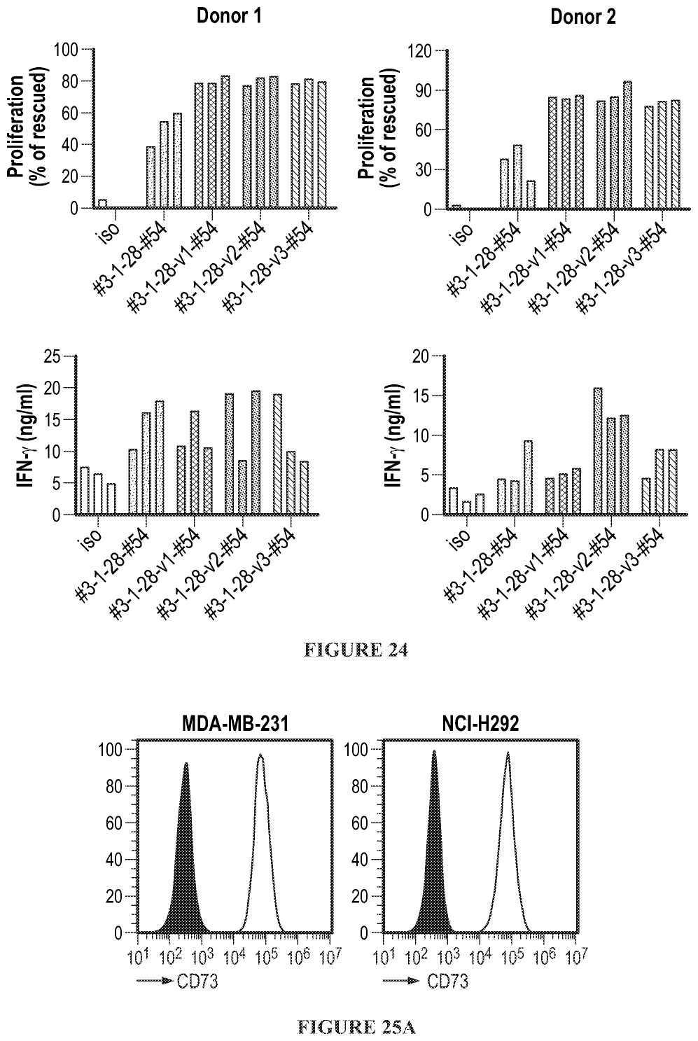

In summary, corresponding to the binding affinity, affinity-improved anti-CD73-CD173 bispecific antibodies showed the better rescue of AMP-suppressed T-cell activation (FIG. 24).

### Example 21

### CD73-Dependent T Cell Activation Induced by Anti-CD73-CD137 Bispecific Antibody

Human T cells were isolated using a RosetteSep™ Human T Cell Enrichment Cocktail (STEMCELL Cat. No. 15061). Purified T cells (1×10⁵ cells) were cocultured with CD73-expressing MDA-MB-231 breast and NCI-H292 lung cancer cells (1×10⁴ cells) in the presence of anti-CD3 (OKT3)-coated polybeads at T-cell:bead ratio 1:1. Three days later, T cell activation was measured by IFN-γ production as measured by ELISA.

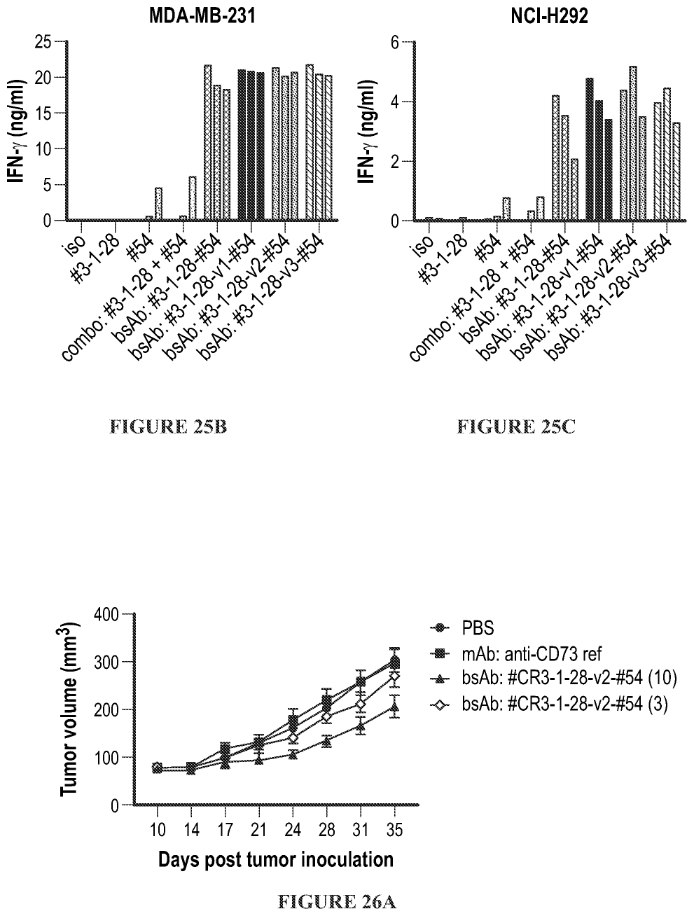

CD73 expression level on MDA-MB-231 breast cancer cells and NCI-H292 lung cancer cells were checked by flow cytometry (FIG. 25A). More robust IFN-γ production was induced by anti-CD73-CD137 bsAb compared to the mono- or combination therapy with anti-CD73 and anti-CD137 #54 antibodies (FIGS. 25B-25C).

### Example 22

### Inhibition of Tumor Growth by Anti-CD73 #3-1-28-V2-CD137 #54 Bispecific Antibody In Vivo

To validate the anti-tumor activity of anti-CD73 #3-1-28-v2-CD137 #54 bsAb, which does not cross-react with mouse CD73 and mouse CD137, human tumor cells (MDA-MB-231) were premixed with human PBMC and xenografted orthotopically to forth mammary fat pad of female SCID-beige mice to evaluate anti-cancer activity in vivo. Ten days post tumor inoculation, equal moles of mAb (M.W. 150 kDa, 10 mg/kg) and bsAb (M.W. 195 kDa, 13 mg/kg) were intraperitoneally injected twice per week. Tumor sizes (mm3) were measured twice per week and calculated as (length×width×width)/2. Tumor inhibition ratio was calculated by the following equations based on the tumor volume (TGIᵥ) or tumor weight (TGI_{w}). TGIᵥ (%)=[1−(Vₜ−V₀ of treatment group)/(Vₜ−V₀ of control group)]×100%, which Vₜ: mean tumor volume after treatment; V₀: mean tumor volume before treatment. TGI_{w} (%)=(mean tumor weight of control group−mean tumor weight of treatment group)/mean tumor weight of control group×100%. For isolation of tumor-infiltrating lymphocytes (TILs), dissected tumors were minced and digested with RPMI-1640 medium containing collagenase IV (Worthington, Cat. No. LS004188), hyaluronidase (Sigma, Cat. No. H6254), and DNase I (Sigma, Cat. No. D5025) by gentleMACS (Miltenyi Biotec Inc.), followed by density gradient separation with Lymphoprep (STEMCELL, Cat. No. 07811). Isolated TILs were stained with cell-type specific markers and analyzed by flow cytometer.

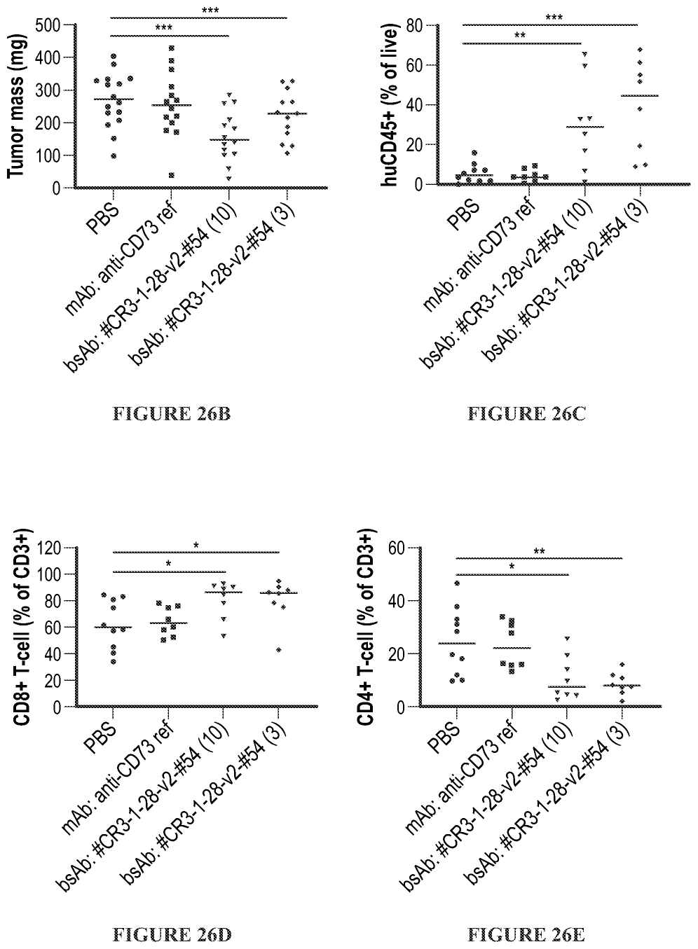

In summary, these results show significant inhibition of tumor growth upon treatment with 10 mg/kg anti-CD73 #3-1-28-v2-CD137 #54 bsAb compared to the treatment with anti-CD73 ref mAb (FIG. 26). The greater tumor inhibition was shown by TGI_{w} as 40.9% and 17.5%, while TGIᵥ was 27% and 10%, at 10 and 3 mg/kg, respectively. Corresponding to the antitumor activity, anti-CD73 #3-1-28-v2-CD137 #54 bsAb specifically induced expansion of CD8+, while decreased CD4+, T-cells in tumors (FIGS. 26A-26E).

### Example 23

### Pharmacokinetic Profile of Anti-CD73-CD137 Bispecific Antibody in Mice

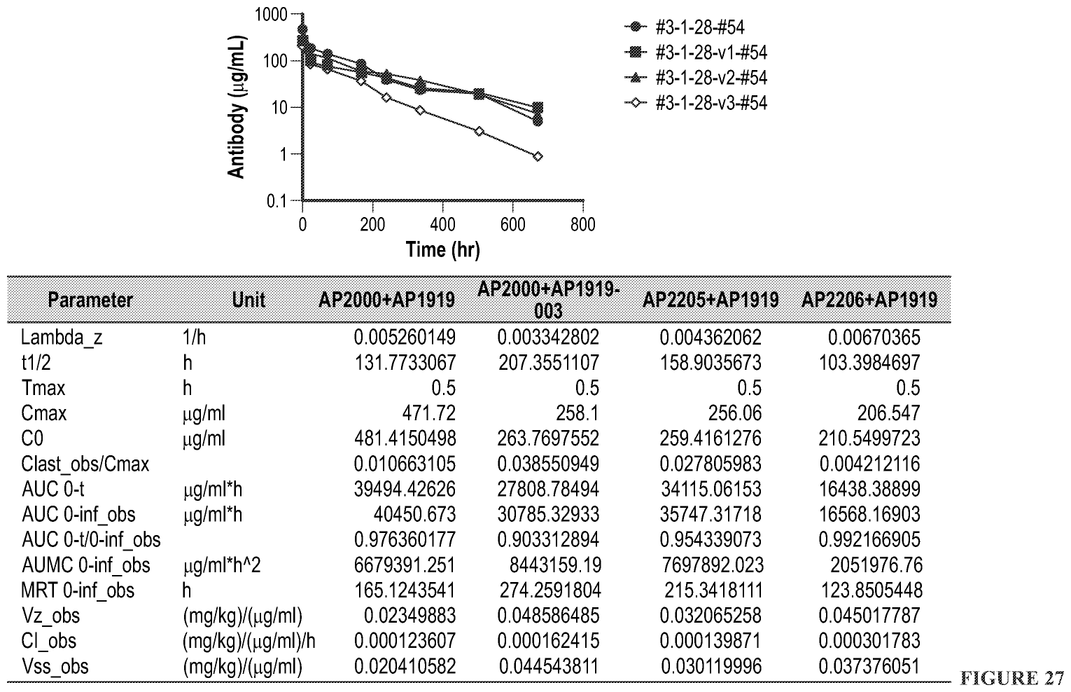

The bispecific anti-CD73 #3-1-28-v2-CD137 #54 antibody (5 mg per kg body weight) was administered via an intravenous bolus injection to SCI-beige mice. Peripheral blood was collected at 0.5, 24, 72, 168, 240, 336, 504, and 672 hours post injection. Plasma concentrations of the antibody were determined by ELISA. MaxiSorp plates (Invitrogen) were coated with CD137-Fc fusion protein (AP Biosciences, 0.5 μg/mL), followed by application of serially diluted plasma samples and anti-CD73-CD137 #54 bsAb as the standard curve. Bound antibodies were detected by CD73-Flag-His antigen (AP Biosciences) and HRP-conjugated anti-Flag antibody using TMB substrate. Plasma antibody concentrations were calculated by the interpolation method. PK parameters were calculated using PKSolver software. Anti-CD73 #3-1-28-v3 showed a worse PK profile with t_{1/2} 103 hours and AUC 16438 μg/ml*h, while anti-CD73 #3-1-28, #3-1-28-v1, #3-1-28-v2-CD137 #54 bsAbs showed a comparable t1/2 and AUC (FIG. 27).

Sequences

| | | |
|---|---|---|
| SEQ ID NO | Name | SEQ (CDR defined by Kabat) |

Anti-p95HER2 monoclonal antibody

| | | |
|---|---|---|
| SEQ ID NO: 1 | Anti-p95HER2 clone R4- | QITLKESGPTLVKPTQTLTLTCTFSGFSLSSGGVGVGWIRQPPGKALE |
| | 3 heavy chain | WLALIYWDDDKRYSPSLKSRLTITKDTSKKQVVLTMTNMDPVDTAT |
| | | YYCAHLDYPENYFDQWGKGTLVTVSS |
| SEQ ID NO: 2 | Anti-p95HER2 clone R4- | DIQMTQSPSSLSASVGDRVTITCRASQSISSYLNWYQQKPGKAPKLLI |
| | 3 light chain | YAASSLQSGVPSRFSGSGSGTDFTLTISSLQPEDFATYYCQQSYSTPTF |
| | | GGGTKVEIKRTVAAPSVFIFPPSDEQLKSGTASVVCLLNNFYPREAKV |
| | | QWKVDNALQSGNSQESVTEQDSKDSTYSLSSTLTLSKADYEKHKLYA |
| | | CEVTHQGLSSPVTKSFNRGEC |
| SEQ ID NO: 3 | CDR-H1 of anti- | SGGVGVG |
| | p95HER2 clone R4-3 | |
| SEQ ID NO: 4 | CDR-H2 of anti- | LIYWDDDKRYSPSLKS |
| | p95HER2 clone R4-3 | |
| SEQ ID NO: 5 | CDR-H3 of anti- | LDYPENYFDQ |
| | p95HER2 clone R4-3 | |
| SEQ ID NO: 6 | CDR-L1 of anti- | RASQSISSYLN |
| | p95HER2 clone R4-3 | |
| SEQ ID NO: 7 | CDR-L2 of anti- | AASSLQS |
| | p95HER2 clone R4-3 | |
| SEQ ID NO: 8 | CDR-L3 of anti- | QQSYSTPT |
| | p95HER2 clone R4-3 | |
| SEQ ID NO: 9 | Anti-p95HER2 clone R4- | QITLKESGPALVKPTQTLTLTCTFSGFSLSTSGVGVGWIRQPPGKALE |
| | 5 heavy chain | WLALIYWDDDKRYSPSLKSRLTITKDTSKNQVVLTMTNMDPVDTAT |
| | | YYCAHSRLPYNSHGYWGQGTTVTVSS |
| SEQ ID NO: 10 | Anti-p95HER2 clone R4- | DIQMTQSPSSLSASVGDRVTITCQASQAIGNYLSWYQQKPGRAPKLLI |
| | 5 light chain | YAASTLHSGVPSRFSGSGSGTDFTLTITSLQPDDFATYYCQQSYSVPV |
| | | TFGLGTKLEIKRTVAAPSVFIFPPSDEQLKSGTASVVCLLNNFYPREAK |
| | | VQWKVDNALQSGNSQESVTEQDSKDSTYSLSSTLTLSKADYEKHKV |
| | | YACEVTHQGLSSPVTKSFNRGEC |
| SEQ ID NO: 11 | CDR-H1 of anti- | TSGVGVG |
| | p95HER2 clone R4-5 | |
| SEQ ID NO: 12 | CDR-H2 of anti- | LIYWDDDKRYSPSLKS |
| | p95HER2 clone R4-5 | |
| SEQ ID NO: 13 | CDR-H3 of an anti- | SRLPYNSHGY |
| | p95HER2 clone R4-5 | |
| SEQ ID NO: 14 | CDR-L1 of anti- | QASQAIGNYLS |
| | p95HER2 clone R4-5 | |
| SEQ ID NO: 15 | CDR-L2 of anti- | AASTLHS |
| | p95HER2 clone R4-5 | |
| SEQ ID NO: 16 | CDR-L3 of anti- | QQSYSVPVT |
| | p95HER2 clone R4-5 | |
| SEQ ID NO: 17 | Anti-p95HER2 clone R4- | QITLKESGPTLVKPTQTLTLTCTFSGFSLSTSGVGVGWIRQPPGKALE |
| | 11 heavy chain | WLALIYWDDDKRYSPSLKSRLTITKDTSKNQVVLTMTNMDPVDTAT |
| | | YYCAHAHPFSGSYNFWGQGTLVTVSS |
| SEQ ID NO: 18 | Anti-p95HER2 clone R4- | DIVMTQSPDSLAVSLGERATINCKSSQSVLESSNNKDYLAWYQQRPG |
| | 11 light chain | QPPKLLIHWASTREPGVPDRFSGSGSGTDFTLTISSLQAEDVAVYYCL |
| | | QSYSNPPTFGQGTKVEIKRTVAAPSVFIFPPSDEQLKSGTASVVCLLNN |
| | | FYPREAKVQWKVDNALQSGNSQESVTEQDSKDSTYSLSSTLTLSKAD |
| | | YEKHKVYACEVTHQGLSSPVTKSFNRGEC |
| SEQ ID NO: 19 | CDR-H1 of anti- | TSGVGVG |
| | p95HER2 clone R4-11 | |
| SEQ ID NO: 20 | CDR-H2 of anti- | LIYWDDDKRYSPSLKS |
| | p95HER2 clone R4-11 | |
| SEQ ID NO: 21 | CDR-H3 of anti- | AHPFSGSYNF |
| | p95HER2 clone R4-11 | |
| SEQ ID NO: 22 | CDR-L1 of anti- | KSSQSVLFSSNNKDYLA |
| | p95HER2 clone R4-11 | |
| SEQ ID NO: 23 | CDR-L2 of anti- | WASTREP |
| | p95HER2 clone R4-11 | |
| SEQ ID NO: 24 | CDR-L3 of anti- | LQSYSNPPT |
| | p95HER2 clone R4-11 | |
| SEQ ID NO: 25 | Anti-p95HER2 clone R4- | QITLKESGPTLVKPTQTLTLTCTFSGFSLSSGGVGVGWIRQPPGKALE |
| | 15 heavy chain | WLALIYWDDDKRYSPSLKSRLTITKDTSKKQVVLTMTNMDPVDTAT |
| | | YYCAHLDYPENYFDQWGQGTLVTVSS |
| SEQ ID NO: 26 | Anti-p95HER2 clone R4- | DIQMTQSPSSLSASVGDRVTFTCRASQSISTYLNWYQQKPGKAPKLLI |
| | 15 light chain | YAASTLQSGVPSRFSGSGSGTDFTLAISSLQPEDFATYFCQQSYSFPTF |
| | | GQGTRLEIKRTMAAPSVFIFPPSDEQLKSGTASVVCLLNNFYPREAKV |
| | | QWKVDNALQSGNSQESVTEQDSKDSTYSLSSTLTLSKADYEKHKVY |
| | | ACEVTHQGLSSPVTKSFNRGEC |
| SEQ ID NO: 27 | CDR-H1 of anti- | SGGVGVG |
| | p95HER2 clone R4-15 | |
| SEQ ID NO: 28 | CDR-H2 of anti- | LIYWDDDKRYSPSLKS |
| | p95HER2 clone R4-15 | |
| SEQ ID NO: 29 | CDR-H3 of anti- | LDYPENYFDQ |
| | p95HER2 clone R4-15 | |
| SEQ ID NO: 30 | CDR-L1 of anti- | RASQSISTYLN |
| | p95HER2 clone R4-15 | |
| SEQ ID NO: 31 | CDR-L2 of anti- | AASTLOS |
| | p95HER2 clone R4-15 | |
| SEQ ID NO: 32 | CDR-L3 of anti- | QQSYSFPT |
| | p95HER2 clone R4-15 | |
| SEQ ID NO: 33 | Anti-p95HER2 clone R4- | QITLKESGPTLVKPTQTLTLTCTFSGFSLSTSGVGVGWIRQHPGKALE |
| | 38 heavy chain | WLALIYWDDDKRYSPSLKSRLTITKDTSKNQVVLTMTNMDPVDTAT |
| | | YYCAHSTVDVHYFDYWGQGTLVTVSS |
| SEQ ID NO: 34 | Anti-p95HER2 clone R4- | DIQMTQSPSSLSASVGDRVTITCRASQSISSYLNWYQQKPGKAPKLLI |
| | 38 light chain | YAASSLQSGVPSRFSGSGSGTDFTLTISSLQPEDFATYYCQQSYSTPTF |
| | | GQGTKVEIKRTVAAPSVFIFPPSDEQLKSGTASVVCLLNNFYPREAKV |
| | | QWKVDNALQSGNSQESVTEQDSKDSTYSLSSTLTLSKADYEKHKVY |
| | | ACEVTHQGLSSPVTKSFNRGEC |
| SEQ ID NO: 35 | CDR-H1 of anti- | TSGVGVG |
| | p95HER2 clone R4-38 | |
| SEQ ID NO: 36 | CDR-H2 of anti- | LIYWDDDKRYSPSLKS |
| | p95HER2 clone R4-38 | |
| SEQ ID NO: 37 | CDR-H3 of anti- | STVDVHYFDY |
| | p95HER2 clone R4-38 | |
| SEQ ID NO: 38 | CDR-L1 of anti- | RASQSISSYLN |
| | p95HER2 clone R4-38 | |
| SEQ ID NO: 39 | CDR-L2 of anti- | AASSLQS |
| | p95HER2 clone R4-38 | |
| SEQ ID NO: 40 | CDR-L3 of anti- | QQSYSTPT |
| | p95HER2 clone R4-38 | |
| SEQ ID NO: 41 | constant domain in heavy | ASTKGPSVFPLAPSSKSTSGGTAALGCLVKDYFPEPVTVSWNSGALTS |
| | chain (IgG1) | GVHTFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDG |
| | | SFFLYSKLTVDKSRWQQGNVFSCSVMHEALHNHYTQKSLSLSPG |
| SEQ ID NO: 42 | constant domain in heavy | ASTKGPSVFPLAPSSKSTSGGTAALGCLVKDYFPEPVTVSWNSGALTS |
| | chain e5 (hIgGle5) | GVHTFPAVLQSSGLYSLSSVVTVPSSSLGTQTYICNVNHKPSNTKVDK |
| | | KVEPKSCDKTHTCPPCPAPELLGGPDVFLFPPKPKDTLMISRTPEVTCV |
| | | VVDVSHEDPEVKFNWYVDGVEVHNAKTKPREEQYNSTYRVVSVLTV |
| | | LHQDWLNGKEYKCKVSNKALPLPEEKTISKAKGQPREPQVYTLPPSR |
| | | DELTKNQVSLTCLVKGFYPSDIAVEWESNGQPENNYKTTPPVLDSDG |
| | | SFFLYSKLTVDKSRWQQGNVFSCSVMHEALHNHYTQKSLSLSPG |
| SEQ ID NO: 43 | SP1 | METDTLLLWVLLLWVPGSTG |
| SEQ ID NO: 44 | GS linker | GGGGS |
| SEQ ID NO: 45 | (G4S)4 linker | GGGGSGGGGSGGGGSGGGGS |

Anti-p95HER2-CD137 bispecific antibody

| | | |
|---|---|---|
| SEQ ID NO: 46 | Anti-p95HER2#4-11- | QITLKESGPTLVKPTQTLTLTCTFSGFSLSTSGVGVGWIRQPPGKALEW |
| | CD137#54 bsAb heavy | LALIYWDDDKRYSPSLKSRLTITKDTSKNQVVLTMTNMDPVDTATYY |
| | chain | CAHAHPFSGSYNFWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPA |
| | | PIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCS |
| | | VMHEALHNHYTQKSLSLSPGGGGGSGGGGSQVQLVQSGAEVKKPGS |
| | | TVKVSCKASGGTFSSYAISWVRQAPGQGLEWMGRIIPILGIANYAQKF |
| | | QGRVTITADKSTSTAYMELSSLRSEDTAVYYCASPPYYDSSGYYPLGA |
| | | FDIWGQGTMVTVSSAGGGGSGGGGSGGGGSGGGGSSYELTQPPSVSV |
| | | SPGQTASITCSGDKLGEKYASWYQQKAGQSPILVIYQDSKRPSGIPERF |
| | | SGSNSGNTATLTISGLQAGDEADYYCQAWDGSSTYVFGTGTKVTVLG |
| SEQ ID NO: 47 | Anti-p95HER2#4-11- | QITLKESGPTLVKPTQTLTLTCTFSGFSLSTSGVGVGWIRQPPGKALEW |
| | CD137#54 bsAb heavy | LALIYWDDDKRYSPSLKSRLTITKDTSKNQVVLTMTNMDPVDTATYY |
| | chain (hIgGle5) | CAHAHPFSGSYNFWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGG |
| | | PDVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPL |
| | | PEEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCS |
| | | VMHEALHNHYTQKSLSLSPGGGGGSGGGGSQVQLVQSGAEVKKPGS |
| | | TVKVSCKASGGTFSSYAISWVRQAPGQGLEWMGRIIPILGIANYAQKF |
| | | QGRVTITADKSTSTAYMELSSLRSEDTAVYYCASPPYYDSSGYYPLGA |
| | | FDIWGQGTMVTVSSAGGGGSGGGGSGGGGSGGGGSSYELTQPPSVSV |
| | | SPGQTASITCSGDKLGEKYASWYQQKAGQSPILVIYQDSKRPSGIPERF |
| | | SGSNSGNTATLTISGLQAGDEADYYCQAWDGSSTYVFGTGTKVTVLG |
| SEQ ID NO: 48 | CD137#54-scFv | QVQLVQSGAEVKKPGSTVKVSCKASGGTFSSYAISWVRQAPGQGLE |
| | | WMGRIIPILGIANYAQKFQGRVTITADKSTSTAYMELSSLRSEDTAVY |
| | | YCASPPYYDSSGYYPLGAFDIWGQGTMVTVSSAGGGGSGGGGSGGG |
| | | GSGGGGSSYELTQPPSVSVSPGQTASITCSGDKLGEKYASWYQQKAG |
| | | QSPIL VIYQDSKRPSGIPERFSGSNSGNTATLTISGLQAGDEADYYCQA |
| | | WDGSSTYVFGTGTKVTVLG |

Anti-CD73 monoclonal antibody

| | | |
|---|---|---|
| SEQ ID NO: 49 | Anti-CD73 clone CR3-4 | QVQLVQSGAEVKKPGSSVKVSCKASGYTFTGYYMHWVRQAPGQGL |
| | heavy chain | EWMGWINPNSGGTNYAQEFQGRVTMTRDTSISTAYMELSRLRSDDT |
| | | AVYYCARESMVRGVSGAFDIWGQGTMVTVSSA |
| SEQ ID NO: 50 | Anti-CD73 clone CR3-4 | QSALTQPASVSGSPGQSITISCTGTSSDVGSYNLVSWYQQHPGKAPKL |
| | light chain | MIYEGSKRPSGVSNRFSGSKSGNTASLTISGLQAEDEADYYCCSYAG |
| | | SSGWDIGTGTKVTVLGQPKANPTVTLFPPSSEELQANKATLVCLISDF |
| | | YPGAVTVAWKADGSPVKAGVETTKPSKQSNNKYAASSYLSLTPEQW |
| | | KSHRSYSCQVTHEGSTVEKTVAPTECS |
| SEQ ID NO: 51 | CDR-H1 of anti-CD73 | GYYMH |
| | clone CR3-4 | |
| SEQ ID NO: 52 | CDR-H2 of anti-CD73 | WINPNSGGTNYAQEFQG |
| | clone CR3-4 | |
| SEQ ID NO: 53 | CDR-H3 of anti-CD73 | ESMVRGVSGAFDI |
| | clone CR3-4 | |
| SEQ ID NO: 54 | CDR-L1 of anti-CD73 | TGTSSDVGSYNLVS |
| | clone CR3-4 | |
| SEQ ID NO: 55 | CDR-L2 of anti-CD73 | EGSKRPS |
| | clone CR3-4 | |
| SEQ ID NO: 56 | CDR-L3 of anti-CD73 | CSYAGSSGWD |
| | clone CR3-4 | |
| SEQ ID NO: 57 | Anti-CD73 clone CR3- | QVQLVQSGAEVKKPGASVKVSCKASGYTFTDFYIHWVRQAPGQGLE |
| | 16 heavy chain | WMGWINPYSGSTNYPQKFQGRVTMTRDTSISTAYMDLSSLTSDDTA |
| | | VYLCVRGRWFDFDYWGQGTTVTVSSA |
| SEQ ID NO: 58 | Anti-CD73 clone CR3- | QSALTQPPSVSGSPGQSITISCTGTSSDVGTYNFVSWYQQHPGKVPKL |
| | 16 light chain | IIYDVSNRPSGVSIRFSGSKSGNTASLTISGLQAEDEADYYCSSFRSGST |
| | | YVFGTGTRVTVLGQPKANPTVTLFPPSSEELQANKATLVCLISDFYPG |
| | | AVTVAWKADGSPVKAGVETTKPSKQSNNKYAASSYLSLTPEQWKSH |
| | | RSYSCQVTHEGSTVEKTVAPAECS |
| SEQ ID NO: 59 | CDR-H1 of anti-CD73 | DFYIH |
| | clone CR3-16 | |
| SEQ ID NO: 60 | CDR-H2 of anti-CD73 | WINPYSGSTNYPQKFQG |
| | clone CR3-16 | |
| SEQ ID NO: 61 | CDR-H3 of anti-CD73 | GRWFDFDY |
| | clone CR3-16 | |
| SEQ ID NO: 62 | CDR-L1 of anti-CD73 | TGTSSDVGTYNFVS |
| | clone CR3-16 | |
| SEQ ID NO: 63 | CDR-L2 of anti-CD73 | DVSNRPS |
| | clone CR3-16 | |
| SEQ ID NO: 64 | CDR-L3 of anti-CD73 | SSFRSGSTYV |
| | clone CR3-16 | |
| SEQ ID NO: 65 | Anti-CD73 clone CR3-1- | QVQLVQSGAEVKKPGSSVKVSCKASGGTFSSYAISWVRQAPGQGLE |
| | 28 heavy chain | WMGRIIPILGIANYAQKFQGRVTITADKSTSTAYMELSSLRSEDTAV |
| | | YYCARDSPVHYYGSGSYYRDAFDIWGQGTMVTVSS |
| SEQ ID NO: 66 | Anti-CD73 clone CR3-1- | QSVLTQPPSASGTPGQRVIISCSGSSSNIGSNSVYWYQQLPGTAPKLLI |
| | 28 light chain | YSNNQRPSGVPGRFSGSKSGTSASLAISGLRSEDEADYYCAAWDDSL |
| | | RGPVFGGGTKLTVLGQPKAAPSVTLFPPSSEELQANKATLVCLISDFY |
| | | PGAVTVAWKADSSPVKAGVETTTPSKQSNNKYAASSYLSLTPEQWKS |
| | | HRSYSCQVTHEGSTVEKTVAPTECS |
| SEQ ID NO: 67 | CDR-H1 of anti-CD73 | SYAIS |
| | clone CR3-1-28 | |
| SEQ ID NO: 68 | CDR-H2 of anti-CD73 | RIIPILGIANYAQKFQG |
| | clone CR3-1-28 | |
| SEQ ID NO: 69 | CDR-H3 of anti-CD73 | DSPVHYYGSGSYYRDAFDI |
| | clone CR3-1-28 | |
| SEQ ID NO: 70 | CDR-L1 of anti-CD73 | SGSSSNIGSNSVY |
| | clone CR3-1-28 | |
| SEQ ID NO: 71 | CDR-L2 of anti-CD73 | SNNQRPS |
| | clone CR3-1-28 | |
| SEQ ID NO: 72 | CDR-L3 of anti-CD73 | AAWDDSLRGPV |
| | clone CR3-1-28 | |
| SEQ ID NO: 73 | Anti-CD73 clone CR3-1- | QVQLVQSGAEVKRPGSSVKVSCKASGGTFSSYAISWVRQAPGQGLE |
| | 37 heavy chain | WMGGIIPIFGTANYAQKFQGRVTITADESTSTAYMELSSLRSEDTAV |
| | | YYCARGVSYYYGMHVWGQGTTVTVSS |
| SEQ ID NO: 74 | Anti-CD73 clone CR3-1- | SYVLTQPPSVSVTPGKTARIACGGNNLESKIVSWYQQKPGQAPVLVIF |
| | 37 light chain | YDTDRPSGISERFSGSNSGNTATLTISRVEAGDEADYYCQVWDSNSD |
| | | HVIFGGGTKLTVLGQPKAAPSVTLFPPSSEELQANKATLVCLISDFYPG |
| | | AVTVAWKADSSPVKAGVETTTPSKQSNNKYAASSYLSLTPEQWKSH |
| | | RSYSCQVTHEGSTVEKTVAPAECS |
| SEQ ID NO: 75 | CDR-H1 of anti-CD73 | SYAIS |
| | clone CR3-1-37 | |
| SEQ ID NO: 76 | CDR-H2 of anti-CD73 | GIIPIFGTANYAQKFQG |
| | clone CR3-1-37 | |
| SEQ ID NO: 77 | CDR-H3 of anti-CD73 | GVSYYYGMHV |
| | clone CR3-1-37 | |
| SEQ ID NO: 78 | CDR-L1 of anti-CD73 | GGNNLESKIVS |
| | clone CR3-1-37 | |
| SEQ ID NO: 79 | CDR-L2 of anti-CD73 | YDTDRPS |
| | clone CR3-1-37 | |
| SEQ ID NO: 80 | CDR-L3 of anti-CD73 | QVWDSNSDHVI |
| | clone CR3-1-37 | |
| SEQ ID NO: 81 | Anti-CD73 clone CR3-1- 28-v1 light chain | 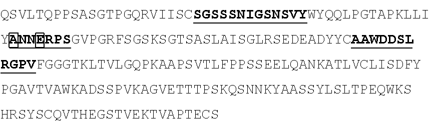 |
| SEQ ID NO: 82 | CDR-L2 of anti-CD73 | ANNERPS |
| | clone CR3-1-28-v1 | |
| SEQ ID NO: 83 | Anti-CD73 clone CR3-1- 28-v2 heavy chain | 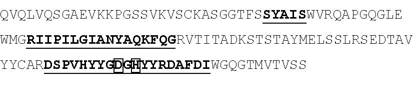 |
| SEQ ID NO: 84 | CDR-H3 of anti-CD73 | DSPVHYYGDGHYYRDAFDI |
| | clone CR3-1-28-v2 | |
| SEQ ID NO: 85 | Anti-CD73 clone CR3-1- 28-v3 heavy chain | 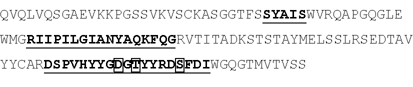 |
| SEQ ID NO: 86 | CDR-H3 of anti-CD73 | DSPVHYYGDGTYYRDSFDI |
| | clone CR3-1-28-v3 | |
| SEQ ID NO: 87 | constant domain in heavy | ASTKGPSVFPLAPSSKSTSGGTAALGCLVKDYFPEPVTVSWNSGALTS |
| | chain (engineered IgG1) | GVHTFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEVTCV |
| | | VVDVSHEDPEVKFNWYVDGVEVHNAKTKPREEQYASTYRVVSVLTV |
| | | LHQDWLNGKEYKCAVSNKALPAPIEKTISKAKGQPREPQVYTLPPSRE |
| | | EMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| SEQ ID NO: 43 | SP1 | METDTLLLWVLLLWVPGSTG |
| SEQ ID NO: 44 | GS linker | GGGGS |
| SEQ ID NO: 160 | (G4S)2 linker | GGGGSGGGGS |

Anti-CD73-CD137 bispecific antibody

| | | |
|---|---|---|
| SEQ ID NO: 88 | Anti-CD73#CR3-1-28- | QVQLVQSGAEVKKPGSSVKVSCKASGGTFSSYAISWVRQAPGQGLE |
| | CD137#54 bsAb heavy | WMGRIIPILGIANYAQKFQGRVTITADKSTSTAYMELSSLRSEDTAVY |
| | chain | YCARDSPVHYYGSGSYYRDAFDIWGQGTMVTVSSASTKGPSVFPLAP |
| | | SSKSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTHTC |
| | | PPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYK |
| | | CAVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDKSR |
| | | WQQGNVFSCSVMHEALHNHYTQKSLSLSPGGGGGSGGGGSQVQLVQ |
| | | SGAEVKKPGSTVKVSCKASGGTFSSYAISWVRQAPGQGLEWMGRIIPI |
| | | LGIANYAQKFQGRVTITADKSTSTAYMELSSLRSEDTAVYYCASPPYY |
| | | DSSGYYPLGAFDIWGQGTMVTVSSAGGGGSGGGGSGGGGSGGGGSS |
| | | YELTQPPSVSVSPGQTASITCSGDKLGEKYASWYQQKAGQSPILVIYQ |
| | | DSKRPSGIPERFSGSNSGNTATLTISGLQAGDEADYYCQAWDGSSTYV |
| | | FGTGTKVTVLG |
| SEQ ID NO: 89 | Anti-CD73 #CR3-1-28- | QVQLVQSGAEVKKPGSSVKVSCKASGGTFSSYAISWVRQAPGQGLE |
| | v2-CD137#54 heavy | WMGRIIPILGIANYAQKFQGRVTITADKSTSTAYMELSSLRSEDTAVY |
| | chain | YCARDSPVHYYGDGHYYRDAFDIWGQGTMVTVSSASTKGPSVFPLA |
| | | PSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHT |
| | | CPPCPAPELLGGPSVFLFPPKPKDTLMISRTPEVTCVVVDVSHEDPEVK |
| | | FNWYVDGVEVHNAKTKPREEQYASTYRVVSVLTVLHQDWLNGKEY |
| | | KCAVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKS |
| | | RWQQGNVFSCSVMHEALHNHYTQKSLSLSPGGGGGSGGGGSQVQLV |
| | | QSGAEVKKPGSTVKVSCKASGGTFSSYAISWVRQAPGQGLEWMGRII |
| | | PILGIANYAQKFQGRVTITADKSTSTAYMELSSLRSEDTAVYYCASPP |
| | | YYDSSGYYPLGAFDIWGQGTMVTVSSAGGGGSGGGGSGGGGSGGGG |
| | | SSYELTQPPSVSVSPGQTASITCSGDKLGEKYASWYQQKAGQSPILVIY |
| | | QDSKRPSGIPERFSGSNSGNTATLTISGLQAGDEADYYCQAWDGSSTY |
| | | VFGTGTKVTVLG |
| SEQ ID NO:90 | Anti-CD73 #CR3-1-28- | QVQLVQSGAEVKKPGSSVKVSCKASGGTFSSYAISWVRQAPGQGLE |
| | v3-CD137#54 heavy | WMGRIIPILGIANYAQKFQGRVTITADKSTSTAYMELSSLRSEDTAVY |
| | chain | YCARDSPVHYYGDGTYYRDSFDIWGQGTMVTVSSASTKGPSVFPLAP |
| | | SSKSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTHTC |
| | | PPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYK |
| | | CAVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDKSR |
| | | WQQGNVFSCSVMHEALHNHYTQKSLSLSPGGGGGSGGGGSQVQLVQ |
| | | SGAEVKKPGSTVKVSCKASGGTFSSYAISWVRQAPGQGLEWMGRIIPI |
| | | LGIANYAQKFQGRVTITADKSTSTAYMELSSLRSEDTAVYYCASPPYY |
| | | DSSGYYPLGAFDIWGQGTMVTVSSAGGGGSGGGGSGGGGSGGGGSS |
| | | YELTQPPSVSVSPGQTASITCSGDKLGEKYASWYQQKAGQSPILVIYQ |
| | | DSKRPSGIPERFSGSNSGNTATLTISGLQAGDEADYYCQAWDGSSTYV |
| | | FGTGTKVTVLG |
| SEQ ID NO:91 | CD137#54-scFv | QVQLVQSGAEVKKPGSTVKVSCKASGGTFSSYAISWVRQAPGQGLE |
| | | WMGRIIPILGIANYAQKFQGRVTITADKSTSTAYMELSSLRSEDTAVY |
| | | YCASPPYYDSSGYYPLGAFDIWGQGTMVTVSSAGGGGSGGGGSGGG |
| | | GSGGGGSSYELTQPPSVSVSPGQTASITCSGDKLGEKYASWYQQKAG |
| | | QSPILVIYQDSKRPSGIPERFSGSNSGNTATLTISGLQAGDEADYYCQA |
| | | WDGSSTYVFGTGTKVTVLG |

### REFERENCES

Allard, B., Allard, D., Buisseret, L., and Stagg, J. (2020). The adenosine pathway in immuno-oncology. Nat Rev Clin Oncol 17, 611-629.
Allard, B., Pommey, S., Smyth, M. J., and Stagg, J. (2013). Targeting CD73 enhances the antitumor activity of anti-PD-1 and anti-CTLA-4 mAbs. Clin Cancer Res 19, 5626-5635.
Anido, J., Scaltriti, M., Bech Serra, J. J., Santiago Josefat, B., Todo, F. R., Baselga, J., and Arribas, J. (2006). Biosynthesis of tumorigenic HER2 C-terminal fragments by alternative initiation of translation. Embo j 25, 3234-3244.
Bacac, M., Colombetti, S., Herter, S., Sam, J., Perro, M., Chen, S., Bianchi, R., Richard, M., Schoenle, A., Nicolini, V., et al. (2018). CD20-TCB with Obinutuzumab Pretreatment as Next-Generation Treatment of Hematologic Malignancies. Clin Cancer Res 24, 4785-4797.
Baselga, J., Carbonell, X., Castañeda-Soto, N.J., Clemens, M., Green, M., Harvey, V., Morales, S., Barton, C., and Ghahramani, P. (2005). Phase II study of efficacy, safety, and pharmacokinetics of trastuzumab monotherapy administered on a 3-weekly schedule. J Clin Oncol 23, 2162-2171.
Baselga, J., Tripathy, D., Mendelsohn, J., Baughman, S., Benz, C. C., Dantis, L., Sklarin, N. T., Seidman, A. D., Hudis, C. A., Moore, J., et al. (1996). Phase II study of weekly intravenous recombinant humanized anti-p185HER2 monoclonal antibody in patients with HER2/neu-overexpressing metastatic breast cancer. J Clin Oncol 14, 737-744.
Burgess, A. W., Cho, H. S., Eigenbrot, C., Ferguson, K. M., Garrett, T. P., Leahy, D. J., Lemmon, M. A., Sliwkowski, M. X., Ward, C. W., and Yokoyama, S. (2003). An open-and-shut case? Recent insights into the activation of EGF/ErbB receptors. Mol Cell 12, 541-552.
Carter, P., Presta, L., Gorman, C. M., Ridgway, J. B., Henner, D., Wong, W. L., Rowland, A. M., Kotts, C., Carver, M. E., and Shepard, H. M. (1992). Humanization of an anti-p185HER2 antibody for human cancer therapy. Proc Natl Acad Sci USA 89, 4285-4289.
Chen, S., Fan, J., Zhang, M., Qin, L., Dominguez, D., Long, A., Wang, G., Ma, R., Li, H., Zhang, Y., et al. (2019). CD73 expression on effector T cells sustained by TGF-beta facilitates tumor resistance to anti-4-1BB/CD137 therapy. Nat Commun 10, 150.
Christianson, T. A., Doherty, J. K., Lin, Y. J., Ramsey, E. E., Holmes, R., Keenan, E. J., and Clinton, G. M. (1998). NH2-terminally truncated HER-2/neu protein: relationship with shedding of the extracellular domain and with prognostic factors in breast cancer. Cancer Res 58, 5123-5129.
Cobleigh, M. A., Vogel, C. L., Tripathy, D., Robert, N.J., Scholl, S., Fehrenbacher, L., Wolter, J. M., Paton, V., Shak, S., Lieberman, G., et al. (1999). Multinational study of the efficacy and safety of humanized anti-HER2 monoclonal antibody in women who have HER2-overexpressing metastatic breast cancer that has progressed after chemotherapy for metastatic disease. J Clin Oncol 17, 2639-2648.
Codony-Servat, J., Albanell, J., Lopez-Talavera, J. C., Arribas, J., and Baselga, J. (1999). Cleavage of the HER2 ectodomain is a pervanadate-activable process that is inhibited by the tissue inhibitor of metalloproteases-1 in breast cancer cells. Cancer Res 59, 1196-1201.
Forte, G., Sorrentino, R., Montinaro, A., Luciano, A., Adcock, I. M., Maiolino, P., Arra, C., Cicala, C., Pinto, A., and Morello, S. (2012). Inhibition of CD73 improves B cell-mediated anti-tumor immunity in a mouse model of melanoma. J Immunol 189, 2226-2233.
Ho M, Kreitman R J, Onda M, Pastan I. (2005) In vitro antibody evolution targeting germline hot spots to increase activity of an anti-CD22 immunotoxin. J Biol Chem 280, 607
Hudziak, R. M., Lewis, G. D., Wingct, M., Fendly, B. M., Shepard, H. M., and Ullrich, A. (1989). p185HER2 monoclonal antibody has antiproliferative effects in vitro and sensitizes human breast tumor cells to tumor necrosis factor. Mol Cell Biol 9, 1165-1172.
Hynes, N. E., and Lanc, H. A. (2005). ERBB receptors and cancer: the complexity of targeted inhibitors. Nat Rev Cancer 5, 341-354.
Jiang, T., Xu, X., Qiao, M., Li, X., Zhao, C., Zhou, F., Gao, G., Wu, F., Chen, X., Su, C., et al. (2018). Comprehensive evaluation of NT5E/CD73 expression and its prognostic significance in distinct types of cancers. BMC Cancer 18, 267.
Lu, Y., Zi, X., Zhao, Y., Mascarenhas, D., and Pollak, M. (2001). Insulin-like growth factor-I receptor signaling and resistance to trastuzumab (Herceptin). J Natl Cancer Inst 93, 1852-1857.
Marty, M., Cognetti, F., Maraninchi, D., Snyder, R., Mauriac, L., Tubiana-Hulin, M., Chan, S., Grimes, D., Antón, A., Lluch, A., et al. (2005). Randomized phase II trial of the efficacy and safety of trastuzumab combined with docetaxel in patients with human epidermal growth factor receptor 2-positive metastatic breast cancer administered as first-line treatment: the M77001 study group. J Clin Oncol 23, 4265-4274.
Molina, M. A., Sácz, R., Ramscy, E. E., Garcia-Barchino, M. J., Rojo, F., Evans, A. J., Albanell, J., Keenan, E. J., Lluch, A., García-Conde, J., et al. (2002). NH (2)-terminal truncated HER-2 protein but not full-length receptor is associated with nodal metastasis in human breast cancer. Clin Cancer Res 8, 347-353.
Nagata, Y., Lan, K. H., Zhou, X., Tan, M., Esteva, F. J., Sahin, A. A., Klos, K. S., Li, P., Monia, B. P., Nguyen, N. T., et al. (2004). PTEN activation contributes to tumor inhibition by trastuzumab, and loss of PTEN predicts trastuzumab resistance in patients. Cancer Cell 6, 117-127.
Neuberger M S, Milstein C. (1995) Somatic hypermutation. Curr Opin Immunol 7, 248
Ohta, A., Kini, R., Ohta, A., Subramanian, M., Madasu, M., and Sitkovsky, M. (2012). The development and immunosuppressive functions of CD4 (+) CD25 (+) FoxP3 (+) regulatory T cells are under influence of the adenosine-A2A adenosine receptor pathway. Front Immunol 3, 190.
Press, M. F., Cordon-Cardo, C., and Slamon, D. J. (1990). Expression of the HER-2/neu proto-oncogene in normal human adult and fetal tissues. Oncogene 5, 953-962.
Qi, X., Li, F., Wu, Y., Cheng, C., Han, P., Wang, J., and Yang, X. (2019). Optimization of 4-1BB antibody for cancer immunotherapy by balancing agonistic strength with FcγR affinity. Nat Commun 10, 2141.
Rius Ruiz, I., Vicario, R., Morancho, B., Morales, C. B., Arenas, E. J., Herter, S., Freimoser-Grundschober, A., Somandin, J., Sam, J., Ast, O., et al. (2018). p95HER2-T cell bispecific antibody for breast cancer treatment. Sci Transl Med 10.
Román, M., Rius-Ruiz, I., Grinyó-Escuer, A., Duro-Sánchez, S., Escorihuela, M., Moessner, E., Klein, C., and Arribas, J. (2022). Humanized CAR T cells targeting p95HER2. bioRxiv, 2022.2005.2020.492812.
Slamon, D. J., Clark, G. M., Wong, S. G., Levin, W. J., Ullrich, A., and McGuire, W. L. (1987). Human breast cancer: correlation of relapse and survival with amplification of the HER-2/neu oncogene. Science 235, 177-182.
Slamon, D. J., Leyland-Jones, B., Shak, S., Fuchs, H., Paton, V., Bajamonde, A., Fleming, T., Eiermann, W., Wolter, J., Pegram, M., et al. (2001). Use of chemotherapy plus a monoclonal antibody against HER2 for metastatic breast cancer that overexpresses HER2. N Engl J Med 344, 783-792.
Stagg, J., Beavis, P. A., Divisckera, U., Liu, M. C., Moller, A., Darcy, P. K., and Smyth, M. J. (2012). CD73-deficient mice are resistant to carcinogenesis. Cancer Res 72, 2190-2196.
Stagg, J., Divisekera, U., Duret, H., Sparwasser, T., Teng, M.W., Darcy, P. K., and Smyth, M. J. (2011). CD73-deficient mice have increased antitumor immunity and are resistant to experimental metastasis. Cancer Res 71, 2892-2900.
Stagg, J., Divisckera, U., Mclaughlin, N., Sharkey, J., Pommey, S., Denoyer, D., Dwyer, K. M., and Smyth, M. J. (2010). Anti-CD73 antibody therapy inhibits breast tumor growth and metastasis. Proc Natl Acad Sci USA 107, 1547-1552.
Tolcher, A. W., Sznol, M., Hu-Licskovan, S., Papadopoulos, K. P., Patnaik, A., Rasco, D. W., Di Gravio, D., Huang, B., Gambhirc, D., Chen, Y., et al. (2017). Phase Ib Study of Utomilumab (PF-05082566), a 4-1BB/CD137 Agonist, in Combination with Pembrolizumab (MK-3475) in Patients with Advanced Solid Tumors. Clin Cancer Res 23, 5349-5357.
Turiello, R., Capone, M., Giannarelli, D., Morretta, E., Monti, M. C., Madonna, G., Mallardo, D., Festino, L., Azzaro, R., Levesque, M. P., et al. (2020). Serum CD73 is a prognostic factor in patients with metastatic melanoma and is associated with response to anti-PD-1 therapy. J Immunother Cancer 8.
Vijayan, D., Young, A., Teng, M. W. L., and Smyth, M. J. (2017). Targeting immunosuppressive adenosine in cancer. Nat Rev Cancer 17, 709-724.
Vogel, C. L., Cobleigh, M. A., Tripathy, D., Gutheil, J. C., Harris, L. N., Fehrenbacher, L., Slamon, D. J., Murphy, M., Novotny, W. F., Burchmore, M., et al. (2002). Efficacy and safety of trastuzumab as a single agent in first-line treatment of HER2-overexpressing metastatic breast cancer. J Clin Oncol 20, 719-726.
Yakes, F. M., Chinratanalab, W., Ritter, C. A., King, W., Seelig, S., and Arteaga, C. L. (2002). Herceptin-induced inhibition of phosphatidylinositol-3 kinase and Akt Is required for antibody-mediated effects on p27, cyclin D1, and antitumor action. Cancer Res 62, 4132-4141.
Yarden, Y., Baselga, J., and Miles, D. (2004). Molecular approach to breast cancer treatment. Semin Oncol 31, 6-13.
Young, A., Ngiow, S. F., Gao, Y., Patch, A. M., Barkauskas, D. S., Messaoudene, M., Lin, G., Coudert, J. D., Stannard, K. A., Zitvogel, L., et al. (2018). A2AR Adenosine Signaling Suppresses Natural Killer Cell Maturation in the Tumor Microenvironment. Cancer Res 78, 1003-1016.
Zhang, Y., Huo, M., Zhou, J., and Xie, S. (2010). PKSolver: An add-in program for pharmacokinetic and pharmacodynamic data analysis in Microsoft Excel. Comput Methods Programs Biomed 99, 306-314.

Although the invention has been described with reference to the above examples, it will be understood that modifications and variations are encompassed within the spirit and scope of the invention. Accordingly, the invention is limited only by the following claims.

Upon entry of the present amendment, the claims will stand as follows in the present application:

1. An antibody or antigen binding fragment thereof comprising:
(i) a V_{H} region comprising complementarity-determining region (CDR)-H1, CDR-H2, and CDR-H3, wherein CDR-H1 is set forth in SEQ ID NO:3, CDR-H2 is set forth in SEQ ID NO:4, and CDR-H3 is set forth in SEQ ID NO:5 or sequences having 90% identity to SEQ ID NO:3, 4 or 5 and the antigen binding specificity thereof; and a V_{L} region comprising CDR-L1, CDR-L2, and CDR-L3, wherein CDR-L1 is set forth in SEQ ID NO:6, CDR-L2 is set forth in SEQ ID NO:7, and CDR-L3 is set forth in SEQ ID NO: 8 or sequences having 90% identity to SEQ ID NO:6, 7 or 8 and the antigen binding specificity thereof;
(ii) a V_{H} region comprising CDR-H1, CDR-H2, and CDR-H3, wherein CDR-H1 is set forth in SEQ ID NO:11, CDR-H2 is set forth in SEQ ID NO:12, and CDR-H3 is set forth in SEQ ID NO:13 or sequences having 90% identity to SEQ ID NO:11, 12 or 13 and the antigen binding specificity thereof; and a V_{L} region comprising CDR-L1, CDR-L2, and CDR-L3, wherein CDR-L1 is set forth in SEQ ID NO:14, CDR-L2 is set forth in SEQ ID NO: 15, and CDR-L3 is set forth in SEQ ID NO:16 or sequences having 90% identity to SEQ ID NO:14, 15 or 16 and the antigen binding specificity thereof;
(iii) a V_{H} region comprising CDR-H1, CDR-H2, and CDR-H3, wherein CDR-H1 is set forth in SEQ ID NO:19, CDR-H2 is set forth in SEQ ID NO:20, and CDR-H3 is set forth in SEQ ID NO:21 or sequences having 90% identity to SEQ ID NO:19, 20 or 21 and the antigen binding specificity thereof; and a V_{L} region comprising CDR-L1, CDR-L2, and CDR-L3, wherein CDR-L1 is set forth in SEQ ID NO:22, CDR-L2 is set forth in SEQ ID NO:23, and CDR-L3 is set forth in SEQ ID NO:24 or sequences having 90% identity to SEQ ID NO:22, 23 or 24 and the antigen binding specificity thereof;
(iv) a V_{H} region comprising CDR-H1, CDR-H2, and CDR-H3, wherein CDR-H1 is set forth in SEQ ID NO:27, CDR-H2 is set forth in SEQ ID NO:28, and CDR-H3 is set forth in SEQ ID NO:29 or sequences having 90% identity to SEQ ID NO:27, 28 or 29 and the antigen binding specificity thereof; and a V_{L} region comprising CDR-L1, CDR-L2, and CDR-L3, wherein CDR-L1 is set forth in SEQ ID NO:30, CDR-L2 is set forth in SEQ ID NO:31, and CDR-L3 is set forth in SEQ ID NO:32 or sequences having 90% identity to SEQ ID NO:30, 31 or 32 and the antigen binding specificity thereof, or
(v) a V_{H} region comprising CDR-H1, CDR-H2, and CDR-H3, wherein CDR-H1 is set forth in SEQ ID NO:35, CDR-H2 is set forth in SEQ ID NO:36, and CDR-H3 is set forth in SEQ ID NO:37 or sequences having 90% identity to SEQ ID NO:35, 36 or 37 and the antigen binding specificity thereof; and a V_{L} region comprising CDR-L1, CDR-L2, and CDR-L3, wherein CDR-L1 is set forth in SEQ ID NO:38, CDR-L2 is set forth in SEQ ID NO:39, and CDR-L3 is set forth in SEQ ID NO:40 or sequences having 90% identity to SEQ ID NO:38, 39 or 40 and the antigen binding specificity thereof, wherein the antibody or antigen binding fragment binds to p95HER2.

2. The antibody or antigen binding fragment of claim 1, wherein:
(i) the VH region has an amino acid sequence having at least 80% identity to SEQ ID NO: 1 and an antigen binding specificity of SEQ ID NOs: 3-5 and the VL region has an amino acid sequence having at least 80% identity to SEQ ID NO:2 and an antigen binding specificity of SEQ ID NOs: 6-8;
(ii) the VH region has an amino acid sequence having at least 80% identity to SEQ ID NO:9 and an antigen binding specificity of SEQ ID NOs: 11-13 and the VL region has an amino acid sequence having at least 80% identity to SEQ ID NO: 10 and an antigen binding specificity of SEQ ID NOs: 14-16;
(iii) the VH region has an amino acid sequence having at least 80% identity to SEQ ID NO: 17 and an antigen binding specificity of SEQ ID NOs: 19-21 and the VL region has an amino acid sequence having at least 80% identity to SEQ ID NO:18 and an antigen binding specificity of SEQ ID NOs: 22-24;
(iv) the VH region has an amino acid sequence having at least 80% identity to SEQ ID NO:25 and an antigen binding specificity of SEQ ID NOs: 27-29 and the VL region has an amino acid sequence having at least 80% identity to SEQ ID NO:26 and an antigen binding specificity of SEQ ID NOs: 30-32; or
(v) the VH region has an amino acid sequence having at least 80% identity to SEQ ID NO:33 and an antigen binding specificity of SEQ ID NOs: 35-37 and the VL region has an amino acid sequence having at least 80% identity to SEQ ID NO:34 and an antigen binding specificity of SEQ ID NOs: 38-40.

3. The antibody or antigen binding fragment of claim 1, wherein:
(i) the VH region is set forth in SEQ ID NO: 1 and the VL region is set forth in SEQ ID NO:2 or a sequence having 90% identity thereto and the binding specificity thereof;
(ii) the VH region is set forth in SEQ ID NO:9 and the VL region is set forth in SEQ ID NO:10 or a sequence having 90% identity thereto and the binding specificity thereof;
(iii) the VH region is set forth in SEQ ID NO:17 and the VL region is set forth in SEQ ID NO:18 or a sequence having 90% identity thereto and the binding specificity thereof;
(iv) the VH region is set forth in SEQ ID NO:25 and the VL region is set forth in SEQ ID NO:26 or a sequence having 90% identity thereto and the binding specificity thereof; or
(v) the VH region is set forth in SEQ ID NO:33 and the VL region is set forth in SEQ ID NO:34 or a sequence having 90% identity thereto and the binding specificity thereof.

4. The antibody or antigen binding fragment of claim 1, wherein the antibody comprises an Fc domain.

5. The antibody or antigen binding fragment of claim 4, wherein the Fc domain is an IgG domain, an IgE domain, an IgM domain, and IgD domain, an IgA domain, or an IgY domain.

6. The antibody or antigen binding fragment of claim 5, wherein the IgG domain is an IgG1 domain, an IgG2 domain, an IgG3 domain, or an IgG4 domain.

7. The antibody or antigen binding fragment of claim 6, wherein the IgG1 domain comprises an amino acid sequence of SEQ ID NO:41.

8. The antibody or antigen binding fragment of claim 6, wherein the IgG1 domain comprises point mutations compared to a wild-type IgG1 domain that modify or enhance antibody-dependent cellular cytotoxicity (ADCC) and/or complement-dependent cytotoxicity (CDC).

9. The antibody or antigen binding fragment of claim 8, wherein the point mutation is S241D, A332L, or I334E.

10. A bispecific antibody comprising a first antigen binding region and a second antigen binding region, wherein the first antigen binding region binds to p95HER2, wherein the antibody comprises a VH region comprising complementarity-determining region (CDR)-H1, CDR-H2, and CDR-H3, wherein CDR-H1 is set forth in SEQ ID NO: 19, CDR-H2 is set forth in SEQ ID NO:20, and CDR-H3 is set forth in SEQ ID NO:21 or sequences having 90% identity to SEQ ID NO:19, 20 or 21 and the antigen binding specificity thereof; and a VL region comprising CDR-L1, CDR-L2, and CDR-L3, wherein CDR-L1 is set forth in SEQ ID NO:22, CDR-L2 is set forth in SEQ ID NO:23, and CDR-L3 is set forth in SEQ ID NO: 24 or sequences having 90% identity to SEQ ID NO:19, 20 or 21 and the antigen binding specificity thereof.

11. (canceled)

12. The bispecific antibody of claim 10, wherein the VH region has an amino acid sequence having at least 80% identity to SEQ ID NO: 17 and an antigen binding specificity of SEQ ID NOs: 19-21 and the VL region has an amino acid sequence having at least 80% identity to SEQ ID NO: 18 and an antigen binding specificity of SEQ ID NOs: 22-24.

13. The bispecific antibody of claim 10, wherein the second antigen binding region binds to an immune checkpoint molecule, or an immune stimulatory molecule.

14. The bispecific antibody of claim 10, wherein the antibody comprises a heavy chain sequence having the amino acid sequence of SEQ ID NO:46 or 47 or a sequence having 90% identity thereto and the binding specificity thereof.

15. The bispecific antibody of claim 14, further comprising a light chain having the amino acid sequence of SEQ ID NO:18.

16-23. (canceled)

24. An antibody-drug conjugate comprising: a therapeutic agent, and the antibody or an antigen binding fragment thereof of claim 1.

25-32. (canceled)

33. An antibody or antigen binding fragment thereof comprising:
(i) a VH region comprising complementarity-determining region (CDR)-H1, CDR-H2, and CDR-H3, wherein CDR-H1 is set forth in SEQ ID NO:51, CDR-H2 is set forth in SEQ ID NO:52, and CDR-H3 is set forth in SEQ ID NO:53 or sequences having 90% identity to SEQ ID NO:51, 52 or 53 and the antigen binding specificity thereof; and a VL region comprising CDR-L1, CDR-L2, and CDR-L3, wherein CDR-L1 is set forth in SEQ ID NO:54, CDR-L2 is set forth in SEQ ID NO:55, and CDR-L3 is set forth in SEQ ID NO: 56 or sequences having 90% identity to SEQ ID NO:55, 56 or 57 and the antigen binding specificity thereof;
(ii) a VH region comprising CDR-H1, CDR-H2, and CDR-H3, wherein CDR-H1 is set forth in SEQ ID NO:59, CDR-H2 is set forth in SEQ ID NO:60, and CDR-H3 is set forth in SEQ ID NO:61 or sequences having 90% identity to SEQ ID NO:59, 60 or 61 and the antigen binding specificity thereof; and a VL region comprising CDR-L1, CDR-L2, and CDR-L3, wherein CDR-L1 is set forth in SEQ ID NO:62, CDR-L2 is set forth in SEQ ID NO:63, and CDR-L3 is set forth in SEQ ID NO:64 or sequences having 90% identity to SEQ ID NO:62, 63 or 64 and the antigen binding specificity thereof;
(iii) a VH region comprising CDR-H1, CDR-H2, and CDR-H3, wherein CDR-H1 is set forth in SEQ ID NO:67, CDR-H2 is set forth in SEQ ID NO:68, and CDR-H3 is set forth in SEQ ID NO:69 or sequences having 90% identity to SEQ ID NO:67, 68 or 69 and the antigen binding specificity thereof; and a VL region comprising CDR-L1, CDR-L2, and CDR-L3, wherein CDR-L1 is set forth in SEQ ID NO:70, CDR-L2 is set forth in SEQ ID NO:71, and CDR-L3 is set forth in SEQ ID NO: 72 or sequences having 90% identity to SEQ ID NO:70, 71 or 72 and the antigen binding specificity thereof;
(iv) a VH region comprising CDR-H1, CDR-H2, and CDR-H3, wherein CDR-H1 is set forth in SEQ ID NO:75, CDR-H2 is set forth in SEQ ID NO:76, and CDR-H3 is set forth in SEQ ID NO:77 or sequences having 90% identity to SEQ ID NO:75, 76 or 77 and the antigen binding specificity thereof; and a VL region comprising CDR-L1, CDR-L2, and CDR-L3, wherein CDR-L1 is set forth in SEQ ID NO:78, CDR-L2 is set forth in SEQ ID NO: 79, and CDR-L3 is set forth in SEQ ID NO:80 or sequences having 90% identity to SEQ ID NO:78, 79 or 80 and the antigen binding specificity thereof;
(v) a VH region comprising CDR-H1, CDR-H2, and CDR-H3, wherein CDR-H1 is set forth in SEQ ID NO:67, CDR-H2 is set forth in SEQ ID NO:68, and CDR-H3 is set forth in SEQ ID NO:69 or sequences having 90% identity to SEQ ID NO:67, 68 or 69 and the antigen binding specificity thereof; and a VL region comprising CDR-L1, CDR-L2, and CDR-L3, wherein CDR-L1 is set forth in SEQ ID NO:70, CDR-L2 is set forth in SEQ ID NO:82, and CDR-L3 is set forth in SEQ ID NO:72 or sequences having 90% identity to SEQ ID NO:70, 82 or 72 and the antigen binding specificity thereof;
(vi) a VH region comprising CDR-H1, CDR-H2, and CDR-H3, wherein CDR-H1 is set forth in SEQ ID NO:67, CDR-H2 is set forth in SEQ ID NO:68, and CDR-H3 is set forth in SEQ ID NO:69 or sequences having 90% identity to SEQ ID NO:67, 68 or 69 and the antigen binding specificity thereof; and a VL region comprising CDR-L1, CDR-L2, and CDR-L3, wherein CDR-L1 is set forth in SEQ ID NO:70, CDR-L2 is set forth in SEQ ID NO:84, and CDR-L3 is set forth in SEQ ID NO: 72 or sequences having 90% identity to SEQ ID NO:70, 84 or 72 and the antigen binding specificity thereof; or
(vii) a VH region comprising CDR-H1, CDR-H2, and CDR-H3, wherein CDR-H1 is set forth in SEQ ID NO:67, CDR-H2 is set forth in SEQ ID NO:68, and CDR-H3 is set forth in SEQ ID NO:69 or sequences having 90% identity to SEQ ID NO:67, 68 or 69 and the antigen binding specificity thereof; and a VL region comprising CDR-L1, CDR-L2, and CDR-L3, wherein CDR-L1 is set forth in SEQ ID NO:70, CDR-L2 is set forth in SEQ ID NO:86, and CDR-L3 is set forth in SEQ ID NO: 72 or sequences having 90% identity to SEQ ID NO:70, 86 or 72 and the antigen binding specificity thereof;
wherein the antibody or antigen binding fragment binds to CD73.

34. The antibody or antigen binding fragment of claim 33, wherein:
(i) the VH region has an amino acid sequence having at least 80% identity to SEQ ID NO: 49 and an antigen binding specificity of SEQ ID NOs: 51-53 and the VL region has an amino acid sequence having at least 80% identity to SEQ ID NO:50 and an antigen binding specificity of SEQ ID NOs: 54-56;
(ii) the VH region has an amino acid sequence having at least 80% identity to SEQ ID NO:57 and an antigen binding specificity of SEQ ID NOs: 59-61 and the VL region has an amino acid sequence having at least 80% identity to SEQ ID NO:58 and an antigen binding specificity of SEQ ID NOs: 62-64;
(iii) the VH region has an amino acid sequence having at least 80% identity to SEQ ID NO:65 and an antigen binding specificity of SEQ ID NOs: 67-69 and the VL region has an amino acid sequence having at least 80% identity to SEQ ID NO:66 and an antigen binding specificity of SEQ ID NOs: 70-72;
(iv) the VH region has an amino acid sequence having at least 80% identity to SEQ ID NO:73 and an antigen binding specificity of SEQ ID NOs: 75-77 and the VL region has an amino acid sequence having at least 80% identity to SEQ ID NO:74 and an antigen binding specificity of SEQ ID NOs: 78-80;
(v) the VH region has an amino acid sequence having at least 80% identity to SEQ ID NO:65 and an antigen binding specificity of SEQ ID NOs: 67-69 and the VL region has an amino acid sequence having at least 80% identity to SEQ ID NO:81 and an antigen binding specificity of SEQ ID NOs: 70, 82 and 72;
(vi) the VH region has an amino acid sequence having at least 80% identity to SEQ ID NO:65 and an antigen binding specificity of SEQ ID NOs: 67-69 and the VL region has an amino acid sequence having at least 80% identity to SEQ ID NO:83 and an antigen binding specificity of SEQ ID NOs: 70, 84 and 72; or
(vii) the VH region has an amino acid sequence having at least 80% identity to SEQ ID NO:65 and an antigen binding specificity of SEQ ID NOs: 67-69 and the VL region has an amino acid sequence having at least 80% identity to SEQ ID NO:85 and an antigen binding specificity of SEQ ID NOs: 70, 86 and 72.

35. The antibody or antigen binding fragment of claim 33, wherein:
(i) the VH region is set forth in SEQ ID NO:49 and the VL region is set forth in SEQ ID NO:50 or a sequence having 90% identity thereto and the binding specificity thereof;
(ii) the VH region is set forth in SEQ ID NO:57 and the VL region is set forth in SEQ ID NO:58 or a sequence having 90% identity thereto and the binding specificity thereof;
(iii) the VH region is set forth in SEQ ID NO:65 and the VL region is set forth in SEQ ID NO:66 or a sequence having 90% identity thereto and the binding specificity thereof;
(iv) the VH region is set forth in SEQ ID NO:73 and the VL region is set forth in SEQ ID NO:74 or a sequence having 90% identity thereto and the binding specificity thereof;
(v) the VH region is set forth in SEQ ID NO:65 and the VL region is set forth in SEQ ID NO:81 or a sequence having 90% identity thereto and the binding specificity thereof;
(vi) the VH region is set forth in SEQ ID NO:65 and the VL region is set forth in SEQ ID NO:83 or a sequence having 90% identity thereto and the binding specificity thereof; or
(vii) the VH region is set forth in SEQ ID NO:65 and the VL region is set forth in SEQ ID NO:85 or a sequence having 90% identity thereto and the binding specificity thereof.

36-41. (canceled)

42. A bispecific antibody comprising a first antigen binding region and a second antigen binding region, wherein the first antigen binding region binds to CD73, wherein the antibody comprises:
(i) a VH region comprising complementarity-determining region (CDR)-H1, CDR-H2, and CDR-H3, wherein CDR-H1 is set forth in SEQ ID NO:67, CDR-H2 is set forth in SEQ ID NO: 68, and CDR-H3 is set forth in SEQ ID NO:69 or sequences having 90% identity to SEQ ID NO: 67, 68 or 69 and the antigen binding specificity thereof; and a VL region comprising CDR-L1, CDR-L2, and CDR-L3, wherein CDR-L1 is set forth in SEQ ID NO:70, CDR-L2 is set forth in SEQ ID NO:71, and CDR-L3 is set forth in SEQ ID NO:72 or sequences having 90% identity to SEQ ID NO:70, 71 or 72 and the antigen binding specificity thereof;
(ii) a VH region comprising complementarity-determining region (CDR)-H1, CDR-H2, and CDR-H3, wherein CDR-H1 is set forth in SEQ ID NO:67, CDR-H2 is set forth in SEQ ID NO: 68, and CDR-H3 is set forth in SEQ ID NO:69 or sequences having 90% identity to SEQ ID NO: 67, 68 or 69 and the antigen binding specificity thereof; and a VL region comprising CDR-L1, CDR-L2, and CDR-L3, wherein CDR-L1 is set forth in SEQ ID NO:70, CDR-L2 is set forth in SEQ ID NO:82, and CDR-L3 is set forth in SEQ ID NO:72 or sequences having 90% identity to SEQ ID NO: 70, 82 or 72 and the antigen binding specificity thereof;
(iii) a VH region comprising complementarity-determining region (CDR)-H1, CDR-H2, and CDR-H3, wherein CDR-H1 is set forth in SEQ ID NO:67, CDR-H2 is set forth in SEQ ID NO: 68, and CDR-H3 is set forth in SEQ ID NO:69 or sequences having 90% identity to SEQ ID NO: 67, 68 or 69 and the antigen binding specificity thereof; and a VL region comprising CDR-L1, CDR-L2, and CDR-L3, wherein CDR-L1 is set forth in SEQ ID NO:70, CDR-L2 is set forth in SEQ ID NO:84, and CDR-L3 is set forth in SEQ ID NO: 72 or sequences having 90% identity to SEQ ID NO: 70, 84 or 72 and the antigen binding specificity thereof; or
(iv) a VH region comprising complementarity-determining region (CDR)-H1, CDR-H2, and CDR-H3, wherein CDR-H1 is set forth in SEQ ID NO:67, CDR-H2 is set forth in SEQ ID NO: 68, and CDR-H3 is set forth in SEQ ID NO:69 or sequences having 90% identity to SEQ ID NO: 67, 68 or 69 and the antigen binding specificity thereof; and a VL region comprising CDR-L1, CDR-L2, and CDR-L3, wherein CDR-L1 is set forth in SEQ ID NO:70, CDR-L2 is set forth in SEQ ID NO:86, and CDR-L3 is set forth in SEQ ID NO:72 or sequences having 90% identity to SEQ ID NO:70, 86 or 72 and the antigen binding specificity thereof.

43. (canceled)

44. The bispecific antibody of claim 42, wherein:
(i) the VH region has an amino acid sequence having at least 80% identity to SEQ ID NO: 65 and an antigen binding specificity of SEQ ID NOs: 67-69 and the VL region has an amino acid sequence having at least 80% identity to SEQ ID NO:66 and an antigen binding specificity of SEQ ID NOs: 70-72;
(ii) the VH region has an amino acid sequence having at least 80% identity to SEQ ID NO:65 and an antigen binding specificity of SEQ ID NOs: 67-69 and the VL region has an amino acid sequence having at least 80% identity to SEQ ID NO:81 and an antigen binding specificity of SEQ ID NOs: 70, 82 and 72;
(iii) the VH region has an amino acid sequence having at least 80% identity to SEQ ID NO:65 and an antigen binding specificity of SEQ ID NOs: 67-69 and the VL region has an amino acid sequence having at least 80% identity to SEQ ID NO:82 and an antigen binding specificity of SEQ ID NOs: 70, 84 and 72; or
(iv) the VH region has an amino acid sequence having at least 80% identity to SEQ ID NO:65 and an antigen binding specificity of SEQ ID NOs: 67-69 and the VL region has an amino acid sequence having at least 80% identity to SEQ ID NO:85 and an antigen binding specificity of SEQ ID NOs: 70, 86 and 72.

45. The bispecific antibody of claim 42, wherein the second antigen binding region binds to an immune checkpoint molecule, or an immune stimulatory molecule.

46. The bispecific antibody of claim 42, wherein the antibody comprises a heavy chain sequence having the amino acid sequence of SEQ ID NO:88, 89, 90 or 91 or a sequence having 90% identity thereto and the binding specificity thereof.

47. The bispecific antibody of claim 42, further comprising a light chain having the amino acid sequence of SEQ ID NO:66.

48-55. (canceled)

56. An antibody-drug conjugate comprising: a therapeutic agent, and the antibody or an antigen binding fragment thereof of claim 33.

57-64. (canceled)

65. An antibody-drug conjugate comprising: a therapeutic agent, and the bispecific antibody of claim 10.

66. An antibody-drug conjugate comprising: a therapeutic agent, and the bispecific antibody of claim 42.


SEQUENCE LISTING (160 sequences)

SEQ ID NO: 1 - protein, 120 aa, synthetic construct
  REGION 1..120: note = Anti-p95HER2 clone R4-3 heavy chain
QITLKESGPTLVKPTQTLTLTCTFSGFSLSSGGVGVGWIRQPPGKALEWLALIYWDDDKRYSPSLKSRLTITKDTSKKQVVLTMTNMDPVDTATYYCAHLDYPENYFDQWGKGTLVTVSS

SEQ ID NO: 2 - protein, 213 aa, synthetic construct
  REGION 1..213: note = Anti-p95HER2 clone R4-3 light chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 3 - protein, 7 aa, synthetic construct
  REGION 1..7: note = CDR-H1 of anti-p95HER2 clone R4-3
SGGVGVG

SEQ ID NO: 4 - protein, 16 aa, synthetic construct
  REGION 1..16: note = CDR-H2 of anti-p95HER2 clone R4-3
LIYWDDDKRYSPSLKS

SEQ ID NO: 5 - protein, 10 aa, synthetic construct
  REGION 1..10: note = CDR-H3 of anti-p95HER2 clone R4-3
LDYPENYFDQ

SEQ ID NO: 6 - protein, 11 aa, synthetic construct
  REGION 1..11: note = CDR-L1 of anti-p95HER2 clone R4-3
RASQSISSYLN

SEQ ID NO: 7 - protein, 7 aa, synthetic construct
  REGION 1..7: note = CDR-L2 of anti-p95HER2 clone R4-3
AASSLQS

SEQ ID NO: 8 - protein, 8 aa, synthetic construct
  REGION 1..8: note = CDR-L3 of anti-p95HER2 clone R4-3
QQSYSTPT

SEQ ID NO: 9 - protein, 120 aa, synthetic construct
  REGION 1..120: note = Anti-p95HER2 clone R4-5 heavy chain
QITLKESGPALVKPTQTLTLTCTFSGFSLSTSGVGVGWIRQPPGKALEWLALIYWDDDKRYSPSLKSRLTITKDTSKNQVVLTMTNMDPVDTATYYCAHSRLPYNSHGYWGQGTTVTVSS

SEQ ID NO: 10 - protein, 214 aa, synthetic construct
  REGION 1..214: note = Anti-p95HER2 clone R4-5 light chain
DIQMTQSPSSLSASVGDRVTITCQASQAIGNYLSWYQQKPGRAPKLLIYAASTLHSGVPSRFSGSGSGTDFTLTITSLQPDDFATYYCQQSYSVPVTFGLGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 7 aa, synthetic construct
  REGION 1..7: note = CDR-H1 of anti-p95HER2 clone R4-5
TSGVGVG

SEQ ID NO: 12 - protein, 16 aa, synthetic construct
  REGION 1..16: note = CDR-H2 of anti-p95HER2 clone R4-5
LIYWDDDKRYSPSLKS

SEQ ID NO: 13 - protein, 10 aa, synthetic construct
  REGION 1..10: note = CDR-H3 of an anti-p95HER2 clone R4-5
SRLPYNSHGY

SEQ ID NO: 14 - protein, 11 aa, synthetic construct
  REGION 1..11: note = CDR-L1 of anti-p95HER2 clone R4-5
QASQAIGNYLS

SEQ ID NO: 15 - protein, 7 aa, synthetic construct
  REGION 1..7: note = CDR-L2 of anti-p95HER2 clone R4-5
AASTLHS

SEQ ID NO: 16 - protein, 9 aa, synthetic construct
  REGION 1..9: note = CDR-L3 of anti-p95HER2 clone R4-5
QQSYSVPVT

SEQ ID NO: 17 - protein, 120 aa, synthetic construct
  REGION 1..120: note = Anti-p95HER2 clone R4-11 heavy chain
QITLKESGPTLVKPTQTLTLTCTFSGFSLSTSGVGVGWIRQPPGKALEWLALIYWDDDKRYSPSLKSRLTITKDTSKNQVVLTMTNMDPVDTATYYCAHAHPFSGSYNFWGQGTLVTVSS

SEQ ID NO: 18 - protein, 220 aa, synthetic construct
  REGION 1..220: note = Anti-p95HER2 clone R4-11 light chain
DIVMTQSPDSLAVSLGERATINCKSSQSVLFSSNNKDYLAWYQQRPGQPPKLLIHWASTREPGVPDRFSGSGSGTDFTLTISSLQAEDVAVYYCLQSYSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 7 aa, synthetic construct
  REGION 1..7: note = CDR-H1 of anti-p95HER2 clone R4-11
TSGVGVG

SEQ ID NO: 20 - protein, 16 aa, synthetic construct
  REGION 1..16: note = CDR-H2 of anti-p95HER2 clone R4-11
LIYWDDDKRYSPSLKS

SEQ ID NO: 21 - protein, 10 aa, synthetic construct
  REGION 1..10: note = CDR-H3 of anti-p95HER2 clone R4-11
AHPFSGSYNF

SEQ ID NO: 22 - protein, 17 aa, synthetic construct
  REGION 1..17: note = CDR-L1 of anti-p95HER2 clone R4-11
KSSQSVLFSSNNKDYLA

SEQ ID NO: 23 - protein, 7 aa, synthetic construct
  REGION 1..7: note = CDR-L2 of anti-p95HER2 clone R4-11
WASTREP

SEQ ID NO: 24 - protein, 9 aa, synthetic construct
  REGION 1..9: note = CDR-L3 of anti-p95HER2 clone R4-11
LQSYSNPPT

SEQ ID NO: 25 - protein, 120 aa, synthetic construct
  REGION 1..120: note = Anti-p95HER2 clone R4-15 heavy chain
QITLKESGPTLVKPTQTLTLTCTFSGFSLSSGGVGVGWIRQPPGKALEWLALIYWDDDKRYSPSLKSRLTITKDTSKKQVVLTMTNMDPVDTATYYCAHLDYPENYFDQWGQGTLVTVSS

SEQ ID NO: 26 - protein, 213 aa, synthetic construct
  REGION 1..213: note = Anti-p95HER2 clone R4-15 light chain
DIQMTQSPSSLSASVGDRVTFTCRASQSISTYLNWYQQKPGKAPKLLIYAASTLQSGVPSRFSGSGSGTDFTLAISSLQPEDFATYFCQQSYSFPTFGQGTRLEIKRTMAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 7 aa, synthetic construct
  REGION 1..7: note = CDR-H1 of anti-p95HER2 clone R4-15
SGGVGVG

SEQ ID NO: 28 - protein, 16 aa, synthetic construct
  REGION 1..16: note = CDR-H2 of anti-p95HER2 clone R4-15
LIYWDDDKRYSPSLKS

SEQ ID NO: 29 - protein, 10 aa, synthetic construct
  REGION 1..10: note = CDR-H3 of anti-p95HER2 clone R4-15
LDYPENYFDQ

SEQ ID NO: 30 - protein, 11 aa, synthetic construct
  REGION 1..11: note = CDR-L1 of anti-p95HER2 clone R4-15
RASQSISTYLN

SEQ ID NO: 31 - protein, 7 aa, synthetic construct
  REGION 1..7: note = CDR-L2 of anti-p95HER2 clone R4-15
AASTLQS

SEQ ID NO: 32 - protein, 8 aa, synthetic construct
  REGION 1..8: note = CDR-L3 of anti-p95HER2 clone R4-15
QQSYSFPT

SEQ ID NO: 33 - protein, 120 aa, synthetic construct
  REGION 1..120: note = Anti-p95HER2 clone R4-38 heavy chain
QITLKESGPTLVKPTQTLTLTCTFSGFSLSTSGVGVGWIRQHPGKALEWLALIYWDDDKRYSPSLKSRLTITKDTSKNQVVLTMTNMDPVDTATYYCAHSTVDVHYFDYWGQGTLVTVSS

SEQ ID NO: 34 - protein, 213 aa, synthetic construct
  REGION 1..213: note = Anti-p95HER2 clone R4-38 light chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 7 aa, synthetic construct
  REGION 1..7: note = CDR-H1 of anti-p95HER2 clone R4-38
TSGVGVG

SEQ ID NO: 36 - protein, 16 aa, synthetic construct
  REGION 1..16: note = CDR-H2 of anti-p95HER2 clone R4-38
LIYWDDDKRYSPSLKS

SEQ ID NO: 37 - protein, 10 aa, synthetic construct
  REGION 1..10: note = CDR-H3 of anti-p95HER2 clone R4-38
STVDVHYFDY

SEQ ID NO: 38 - protein, 11 aa, synthetic construct
  REGION 1..11: note = CDR-L1 of anti-p95HER2 clone R4-38
RASQSISSYLN

SEQ ID NO: 39 - protein, 7 aa, synthetic construct
  REGION 1..7: note = CDR-L2 of anti-p95HER2 clone R4-38
AASSLQS

SEQ ID NO: 40 - protein, 8 aa, synthetic construct
  REGION 1..8: note = CDR-L3 of anti-p95HER2 clone R4-38
QQSYSTPT

SEQ ID NO: 41 - protein, 329 aa, synthetic construct
  REGION 1..329: note = constant domain in heavy chain (IgG1)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 329 aa, synthetic construct
  REGION 1..329: note = constant domain in heavy chain e5 (hIgG1e5)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 20 aa, synthetic construct
  REGION 1..20: note = SP1
METDTLLLWVLLLWVPGSTG

SEQ ID NO: 44 - protein, 5 aa, synthetic construct
  REGION 1..5: note = GS linker
GGGGS

SEQ ID NO: 45 - protein, 20 aa, synthetic construct
  REGION 1..20: note = (G4S)4 linker
GGGGSGGGGSGGGGSGGGGS

SEQ ID NO: 46 - protein, 714 aa, synthetic construct
  REGION 1..714: note = Anti-p95HER2#4-11-CD137#54 bsAb heavy chain
QITLKESGPTLVKPTQTLTLTCTFSGFSLSTSGVGVGWIRQPPGKALEWLALIYWDDDKRYSPSLKSRLTITKDTSKNQVVLTMTNMDPVDTATYYCAHAHPFSGSYNFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVQSGAEVKKPGSTVKVSCKASGGTFSSYAISWVRQAPGQGLEWMGRIIPILGIANYAQKFQGRVTITADKSTSTAYMELSSLRSEDTAVYYCASPPYYDSSGYYPLGAFDIWGQGTMVTVSSAGGGGSGGGGSGGGGSGGGGSSYELTQPPSVSVSPGQTASITCSGDKLGEKYASWYQQKAGQSPILVIYQDSKRPSGIPERFSGSNSGNTATLTISGLQAGDEADYYCQAWDGSSTYVFGTGTKVTVLG

SEQ ID NO: 47 - protein, 714 aa, synthetic construct
  REGION 1..714: note = Anti-p95HER2#4-11-CD137#54 bsAb heavy chain (hIgG1e5)
QITLKESGPTLVKPTQTLTLTCTFSGFSLSTSGVGVGWIRQPPGKALEWLALIYWDDDKRYSPSLKSRLTITKDTSKNQVVLTMTNMDPVDTATYYCAHAHPFSGSYNFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVQSGAEVKKPGSTVKVSCKASGGTFSSYAISWVRQAPGQGLEWMGRIIPILGIANYAQKFQGRVTITADKSTSTAYMELSSLRSEDTAVYYCASPPYYDSSGYYPLGAFDIWGQGTMVTVSSAGGGGSGGGGSGGGGSGGGGSSYELTQPPSVSVSPGQTASITCSGDKLGEKYASWYQQKAGQSPILVIYQDSKRPSGIPERFSGSNSGNTATLTISGLQAGDEADYYCQAWDGSSTYVFGTGTKVTVLG

SEQ ID NO: 48 - protein, 255 aa, synthetic construct
  REGION 1..255: note = CD137#54-scFv
QVQLVQSGAEVKKPGSTVKVSCKASGGTFSSYAISWVRQAPGQGLEWMGRIIPILGIANYAQKFQGRVTITADKSTSTAYMELSSLRSEDTAVYYCASPPYYDSSGYYPLGAFDIWGQGTMVTVSSAGGGGSGGGGSGGGGSGGGGSSYELTQPPSVSVSPGQTASITCSGDKLGEKYASWYQQKAGQSPILVIYQDSKRPSGIPERFSGSNSGNTATLTISGLQAGDEADYYCQAWDGSSTYVFGTGTKVTVLG

SEQ ID NO: 49 - protein, 123 aa, synthetic construct
  REGION 1..123: note = Anti-CD73 clone CR3-4 heavy chain
QVQLVQSGAEVKKPGSSVKVSCKASGYTFTGYYMHWVRQAPGQGLEWMGWINPNSGGTNYAQEFQGRVTMTRDTSISTAYMELSRLRSDDTAVYYCARESMVRGVSGAFDIWGQGTMVTVSSA

SEQ ID NO: 50 - protein, 216 aa, synthetic construct
  REGION 1..216: note = Anti-CD73 clone CR3-4 light chain
QSALTQPASVSGSPGQSITISCTGTSSDVGSYNLVSWYQQHPGKAPKLMIYEGSKRPSGVSNRFSGSKSGNTASLTISGLQAEDEADYYCCSYAGSSGWDIGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 51 - protein, 5 aa, synthetic construct
  REGION 1..5: note = CDR-H1 of anti-CD73 clone CR3-4
GYYMH

SEQ ID NO: 52 - protein, 17 aa, synthetic construct
  REGION 1..17: note = CDR-H2 of anti-CD73 clone CR3-4
WINPNSGGTNYAQEFQG

SEQ ID NO: 53 - protein, 13 aa, synthetic construct
  REGION 1..13: note = CDR-H3 of anti-CD73 clone CR3-4
ESMVRGVSGAFDI

SEQ ID NO: 54 - protein, 14 aa, synthetic construct
  REGION 1..14: note = CDR-L1 of anti-CD73 clone CR3-4
TGTSSDVGSYNLVS

SEQ ID NO: 55 - protein, 7 aa, synthetic construct
  REGION 1..7: note = CDR-L2 of anti-CD73 clone CR3-4
EGSKRPS

SEQ ID NO: 56 - protein, 10 aa, synthetic construct
  REGION 1..10: note = CDR-L3 of anti-CD73 clone CR3-4
CSYAGSSGWD

SEQ ID NO: 57 - protein, 118 aa, synthetic construct
  REGION 1..118: note = Anti-CD73 clone CR3-16 heavy chain
QVQLVQSGAEVKKPGASVKVSCKASGYTFTDFYIHWVRQAPGQGLEWMGWINPYSGSTNYPQKFQGRVTMTRDTSISTAYMDLSSLTSDDTAVYLCVRGRWFDFDYWGQGTTVTVSSA

SEQ ID NO: 58 - protein, 216 aa, synthetic construct
  REGION 1..216: note = Anti-CD73 clone CR3-16 light chain
QSALTQPPSVSGSPGQSITISCTGTSSDVGTYNFVSWYQQHPGKVPKLIIYDVSNRPSGVSIRFSGSKSGNTASLTISGLQAEDEADYYCSSFRSGSTYVFGTGTRVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPAECS

SEQ ID NO: 59 - protein, 5 aa, synthetic construct
  REGION 1..5: note = CDR-H1 of anti-CD73 clone CR3-16
DFYIH

SEQ ID NO: 60 - protein, 17 aa, synthetic construct
  REGION 1..17: note = CDR-H2 of anti-CD73 clone CR3-16
WINPYSGSTNYPQKFQG

SEQ ID NO: 61 - protein, 8 aa, synthetic construct
  REGION 1..8: note = CDR-H3 of anti-CD73 clone CR3-16
GRWFDFDY

SEQ ID NO: 62 - protein, 14 aa, synthetic construct
  REGION 1..14: note = CDR-L1 of anti-CD73 clone CR3-16
TGTSSDVGTYNFVS

SEQ ID NO: 63 - protein, 7 aa, synthetic construct
  REGION 1..7: note = CDR-L2 of anti-CD73 clone CR3-16
DVSNRPS

SEQ ID NO: 64 - protein, 10 aa, synthetic construct
  REGION 1..10: note = CDR-L3 of anti-CD73 clone CR3-16
SSFRSGSTYV

SEQ ID NO: 65 - protein, 128 aa, synthetic construct
  REGION 1..128: note = Anti-CD73 clone CR3-1-28 heavy chain
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARDSPVHYYGSGSYYRDAFDIWGQGTMVTVSS

SEQ ID NO: 66 - protein, 216 aa, synthetic construct
  REGION 1..216: note = Anti-CD73 clone CR3-1-28 light chain
QSVLTQPPSASGTPGQRVIISCSGSSSNIGSNSVYWYQQLPGTAPKLLIYSNNQRPSGVPGRFSGSKSGTSASLAISGLRSEDEADYYCAAWDDSLRGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 67 - protein, 5 aa, synthetic construct
  REGION 1..5: note = CDR-H1 of anti-CD73 clone CR3-1-28
SYAIS

SEQ ID NO: 68 - protein, 17 aa, synthetic construct
  REGION 1..17: note = CDR-H2 of anti-CD73 clone CR3-1-28
RIIPILGIANYAQKFQG

SEQ ID NO: 69 - protein, 19 aa, synthetic construct
  REGION 1..19: note = CDR-H3 of anti-CD73 clone CR3-1-28
DSPVHYYGSGSYYRDAFDI

SEQ ID NO: 70 - protein, 13 aa, synthetic construct
  REGION 1..13: note = CDR-L1 of anti-CD73 clone CR3-1-28
SGSSSNIGSNSVY

SEQ ID NO: 71 - protein, 7 aa, synthetic construct
  REGION 1..7: note = CDR-L2 of anti-CD73 clone CR3-1-28
SNNQRPS

SEQ ID NO: 72 - protein, 11 aa, synthetic construct
  REGION 1..11: note = CDR-L3 of anti-CD73 clone CR3-1-28
AAWDDSLRGPV

SEQ ID NO: 73 - protein, 119 aa, synthetic construct
  REGION 1..119: note = Anti-CD73 clone CR3-1-37 heavy chain
QVQLVQSGAEVKRPGSSVKVSCKASGGTFSSYAISWVRQAPGQGLEWMGGIIPIFGTANYAQKFQGRVTITADESTSTAYMELSSLRSEDTAVYYCARGVSYYYGMHVWGQGTTVTVSS

SEQ ID NO: 74 - protein, 214 aa, synthetic construct
  REGION 1..214: note = Anti-CD73 clone CR3-1-37 light chain
SYVLTQPPSVSVTPGKTARIACGGNNLESKIVSWYQQKPGQAPVLVIFYDTDRPSGISERFSGSNSGNTATLTISRVEAGDEADYYCQVWDSNSDHVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 75 - protein, 5 aa, synthetic construct
  REGION 1..5: note = CDR-H1 of anti-CD73 clone CR3-1-37
SYAIS

SEQ ID NO: 76 - protein, 17 aa, synthetic construct
  REGION 1..17: note = CDR-H2 of anti-CD73 clone CR3-1-37
GIIPIFGTANYAQKFQG

SEQ ID NO: 77 - protein, 10 aa, synthetic construct
  REGION 1..10: note = CDR-H3 of anti-CD73 clone CR3-1-37
GVSYYYGMHV

SEQ ID NO: 78 - protein, 11 aa, synthetic construct
  REGION 1..11: note = CDR-L1 of anti-CD73 clone CR3-1-37
GGNNLESKIVS

SEQ ID NO: 79 - protein, 7 aa, synthetic construct
  REGION 1..7: note = CDR-L2 of anti-CD73 clone CR3-1-37
YDTDRPS

SEQ ID NO: 80 - protein, 11 aa, synthetic construct
  REGION 1..11: note = CDR-L3 of anti-CD73 clone CR3-1-37
QVWDSNSDHVI

SEQ ID NO: 81 - protein, 216 aa, synthetic construct
  REGION 1..216: note = Anti-CD73 clone CR3-1-28-v1 light chain
QSVLTQPPSASGTPGQRVIISCSGSSSNIGSNSVYWYQQLPGTAPKLLIYANNERPSGVPGRFSGSKSGTSASLAISGLRSEDEADYYCAAWDDSLRGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 82 - protein, 7 aa, synthetic construct
  REGION 1..7: note = CDR-L2 of anti-CD73 clone CR3-1-28-v1
ANNERPS

SEQ ID NO: 83 - protein, 128 aa, synthetic construct
  REGION 1..128: note = Anti-CD73 clone CR3-1-28-v2 heavy chain
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARDSPVHYYGDGHYYRDAFDIWGQGTMVTVSS

SEQ ID NO: 84 - protein, 19 aa, synthetic construct
  REGION 1..19: note = CDR-H3 of anti-CD73 clone CR3-1-28-v2
DSPVHYYGDGHYYRDAFDI

SEQ ID NO: 85 - protein, 128 aa, synthetic construct
  REGION 1..128: note = Anti-CD73 clone CR3-1-28-v3 heavy chain
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARDSPVHYYGDGTYYRDSFDIWGQGTMVTVSS

SEQ ID NO: 86 - protein, 19 aa, synthetic construct
  REGION 1..19: note = CDR-H3 of anti-CD73 clone CR3-1-28-v3
DSPVHYYGDGTYYRDSFDI

SEQ ID NO: 87 - protein, 329 aa, synthetic construct
  REGION 1..329: note = constant domain in heavy chain (engineered IgG1)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 722 aa, synthetic construct
  REGION 1..722: note = Anti-CD73#CR3-1-28-CD137#54 bsAb heavy chain
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARDSPVHYYGSGSYYRDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVQSGAEVKKPGSTVKVSCKASGGTFSSYAISWVRQAPGQGLEWMGRIIPILGIANYAQKFQGRVTITADKSTSTAYMELSSLRSEDTAVYYCASPPYYDSSGYYPLGAFDIWGQGTMVTVSSAGGGGSGGGGSGGGGSGGGGSSYELTQPPSVSVSPGQTASITCSGDKLGEKYASWYQQKAGQSPILVIYQDSKRPSGIPERFSGSNSGNTATLTISGLQAGDEADYYCQAWDGSSTYVFGTGTKVTVLG

SEQ ID NO: 89 - protein, 722 aa, synthetic construct
  REGION 1..722: note = Anti-CD73 #CR3-1-28- v2-CD137#54 heavy chain
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARDSPVHYYGDGHYYRDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVQSGAEVKKPGSTVKVSCKASGGTFSSYAISWVRQAPGQGLEWMGRIIPILGIANYAQKFQGRVTITADKSTSTAYMELSSLRSEDTAVYYCASPPYYDSSGYYPLGAFDIWGQGTMVTVSSAGGGGSGGGGSGGGGSGGGGSSYELTQPPSVSVSPGQTASITCSGDKLGEKYASWYQQKAGQSPILVIYQDSKRPSGIPERFSGSNSGNTATLTISGLQAGDEADYYCQAWDGSSTYVFGTGTKVTVLG

SEQ ID NO: 90 - protein, 722 aa, synthetic construct
  REGION 1..722: note = Anti-CD73 #CR3-1-28- v3-CD137#54 heavy chain
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARDSPVHYYGDGTYYRDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVQSGAEVKKPGSTVKVSCKASGGTFSSYAISWVRQAPGQGLEWMGRIIPILGIANYAQKFQGRVTITADKSTSTAYMELSSLRSEDTAVYYCASPPYYDSSGYYPLGAFDIWGQGTMVTVSSAGGGGSGGGGSGGGGSGGGGSSYELTQPPSVSVSPGQTASITCSGDKLGEKYASWYQQKAGQSPILVIYQDSKRPSGIPERFSGSNSGNTATLTISGLQAGDEADYYCQAWDGSSTYVFGTGTKVTVLG

SEQ ID NO: 91 - protein, 255 aa, synthetic construct; identical to SEQ ID NO: 48
  REGION 1..255: note = CD137#54-scFv

SEQ ID NO: 92 - protein, 5 aa, synthetic construct
  REGION 1..5: note = linear 5 amino acid peptides with peptide-peptide overlaps
MPIWK

SEQ ID NO: 93 - protein, 5 aa, synthetic construct
  REGION 1..5: note = linear 5 amino acid peptides with peptide-peptide overlaps
PIWKF

SEQ ID NO: 94 - protein, 5 aa, synthetic construct
  REGION 1..5: note = linear 5 amino acid peptides with peptide-peptide overlaps
IWKFP

SEQ ID NO: 95 - protein, 5 aa, synthetic construct
  REGION 1..5: note = linear 5 amino acid peptides with peptide-peptide overlaps
WKFPD

SEQ ID NO: 96 - protein, 5 aa, synthetic construct
  REGION 1..5: note = linear 5 amino acid peptides with peptide-peptide overlaps
KFPDE

SEQ ID NO: 97 - protein, 5 aa, synthetic construct
  REGION 1..5: note = linear 5 amino acid peptides with peptide-peptide overlaps
FPDEE

SEQ ID NO: 98 - protein, 5 aa, synthetic construct
  REGION 1..5: note = linear 5 amino acid peptides with peptide-peptide overlaps
PDEEG

SEQ ID NO: 99 - protein, 5 aa, synthetic construct
  REGION 1..5: note = linear 5 amino acid peptides with peptide-peptide overlaps
DEEGA

SEQ ID NO: 100 - protein, 10 aa, synthetic construct
  REGION 1..10: note = linear 10 amino acid peptides with peptide-peptide overlaps
GSGSGMPIWK

SEQ ID NO: 101 - protein, 10 aa, synthetic construct
  REGION 1..10: note = linear 10 amino acid peptides with peptide-peptide overlaps
SGSGMPIWKF

SEQ ID NO: 102 - protein, 10 aa, synthetic construct
  REGION 1..10: note = linear 10 amino acid peptides with peptide-peptide overlaps
GSGMPIWKFP

SEQ ID NO: 103 - protein, 10 aa, synthetic construct
  REGION 1..10: note = linear 10 amino acid peptides with peptide-peptide overlaps
SGMPIWKFPD

SEQ ID NO: 104 - protein, 10 aa, synthetic construct
  REGION 1..10: note = linear 10 amino acid peptides with peptide-peptide overlaps
GMPIWKFPDE

SEQ ID NO: 105 - protein, 10 aa, synthetic construct
  REGION 1..10: note = linear 10 amino acid peptides with peptide-peptide overlaps
MPIWKFPDEE

SEQ ID NO: 106 - protein, 10 aa, synthetic construct
  REGION 1..10: note = linear 10 amino acid peptides with peptide-peptide overlaps
PIWKFPDEEG

SEQ ID NO: 107 - protein, 10 aa, synthetic construct
  REGION 1..10: note = linear 10 amino acid peptides with peptide-peptide overlaps
IWKFPDEEGA

SEQ ID NO: 108 - protein, 10 aa, synthetic construct
  REGION 1..10: note = linear 10 amino acid peptides with peptide-peptide overlaps
WKFPDEEGAC

SEQ ID NO: 109 - protein, 10 aa, synthetic construct
  REGION 1..10: note = linear 10 amino acid peptides with peptide-peptide overlaps
KFPDEEGACQ

SEQ ID NO: 110 - protein, 10 aa, synthetic construct
  REGION 1..10: note = linear 10 amino acid peptides with peptide-peptide overlaps
FPDEEGACQP

SEQ ID NO: 111 - protein, 10 aa, synthetic construct
  REGION 1..10: note = linear 10 amino acid peptides with peptide-peptide overlaps
PDEEGACQPC

SEQ ID NO: 112 - protein, 15 aa, synthetic construct
  REGION 1..15: note = linear 15 amino acid peptides with peptide-peptide overlaps
GSGSGSGMPIWKFPD

SEQ ID NO: 113 - protein, 15 aa, synthetic construct
  REGION 1..15: note = linear 15 amino acid peptides with peptide-peptide overlaps
SGSGSGMPIWKFPDE

SEQ ID NO: 114 - protein, 15 aa, synthetic construct
  REGION 1..15: note = linear 15 amino acid peptides with peptide-peptide overlaps
GSGSGMPIWKFPDEE

SEQ ID NO: 115 - protein, 15 aa, synthetic construct
  REGION 1..15: note = linear 15 amino acid peptides with peptide-peptide overlaps
SGSGMPIWKFPDEEG

SEQ ID NO: 116 - protein, 15 aa, synthetic construct
  REGION 1..15: note = linear 15 amino acid peptides with peptide-peptide overlaps
GSGMPIWKFPDEEGA

SEQ ID NO: 117 - protein, 15 aa, synthetic construct
  REGION 1..15: note = linear 15 amino acid peptides with peptide-peptide overlaps
SGMPIWKFPDEEGAC

SEQ ID NO: 118 - protein, 15 aa, synthetic construct
  REGION 1..15: note = linear 15 amino acid peptides with peptide-peptide overlaps
GMPIWKFPDEEGACQ

SEQ ID NO: 119 - protein, 15 aa, synthetic construct
  REGION 1..15: note = linear 15 amino acid peptides with peptide-peptide overlaps
MPIWKFPDEEGACQP

SEQ ID NO: 120 - protein, 15 aa, synthetic construct
  REGION 1..15: note = linear 15 amino acid peptides with peptide-peptide overlaps
PIWKFPDEEGACQPC

SEQ ID NO: 121 - protein, 15 aa, synthetic construct
  REGION 1..15: note = linear 15 amino acid peptides with peptide-peptide overlaps
IWKFPDEEGACQPCP

SEQ ID NO: 122 - protein, 15 aa, synthetic construct
  REGION 1..15: note = linear 15 amino acid peptides with peptide-peptide overlaps
WKFPDEEGACQPCPI

SEQ ID NO: 123 - protein, 15 aa, synthetic construct
  REGION 1..15: note = linear 15 amino acid peptides with peptide-peptide overlaps
KFPDEEGACQPCPIN

SEQ ID NO: 124 - protein, 15 aa, synthetic construct
  REGION 1..15: note = linear 15 amino acid peptides with peptide-peptide overlaps
FPDEEGACQPCPINC

SEQ ID NO: 125 - protein, 5 aa, synthetic construct
  REGION 1..5: note = peptide with the consensus motif
GSGSG

SEQ ID NO: 126 - protein, 5 aa, synthetic construct
  REGION 1..5: note = peptide with the consensus motif
SGSGM

SEQ ID NO: 127 - protein, 5 aa, synthetic construct
  REGION 1..5: note = peptide with the consensus motif
GMPIW

SEQ ID NO: 128 - protein, 5 aa, synthetic construct
  REGION 1..5: note = peptide with the consensus motif
EEGAC

SEQ ID NO: 129 - protein, 5 aa, synthetic construct
  REGION 1..5: note = peptide with the consensus motif
ACQPC

SEQ ID NO: 130 - protein, 5 aa, synthetic construct
  REGION 1..5: note = peptide with the consensus motif
PCPIN

SEQ ID NO: 131 - protein, 5 aa, synthetic construct
  REGION 1..5: note = peptide with the consensus motif
INCTH

SEQ ID NO: 132 - protein, 5 aa, synthetic construct
  REGION 1..5: note = peptide with the consensus motif
THSCV

SEQ ID NO: 133 - protein, 5 aa, synthetic construct
  REGION 1..5: note = peptide with the consensus motif
CVDLD

SEQ ID NO: 134 - protein, 5 aa, synthetic construct
  REGION 1..5: note = peptide with the consensus motif
LDDKG

SEQ ID NO: 135 - protein, 5 aa, synthetic construct
  REGION 1..5: note = peptide with the consensus motif
KGCPA

SEQ ID NO: 136 - protein, 5 aa, synthetic construct
  REGION 1..5: note = peptide with the consensus motif
PAEQR

SEQ ID NO: 137 - protein, 5 aa, synthetic construct
  REGION 1..5: note = peptide with the consensus motif
QRASP

SEQ ID NO: 138 - protein, 5 aa, synthetic construct
  REGION 1..5: note = peptide with the consensus motif
SPLTG

SEQ ID NO: 139 - protein, 5 aa, synthetic construct
  REGION 1..5: note = peptide with the consensus motif
TGSGS

SEQ ID NO: 140 - protein, 10 aa, synthetic construct
  REGION 1..10: note = peptide with the consensus motif
DEEGACQPCP

SEQ ID NO: 141 - protein, 10 aa, synthetic construct
  REGION 1..10: note = peptide with the consensus motif
GACQPCPINC

SEQ ID NO: 142 - protein, 10 aa, synthetic construct
  REGION 1..10: note = peptide with the consensus motif
QPCPINCTHS

SEQ ID NO: 143 - protein, 10 aa, synthetic construct
  REGION 1..10: note = peptide with the consensus motif
PINCTHSCVD

SEQ ID NO: 144 - protein, 10 aa, synthetic construct
  REGION 1..10: note = peptide with the consensus motif
CTHSCVDLDD

SEQ ID NO: 145 - protein, 10 aa, synthetic construct
  REGION 1..10: note = peptide with the consensus motif
SCVDLDDKGC

SEQ ID NO: 146 - protein, 10 aa, synthetic construct
  REGION 1..10: note = peptide with the consensus motif
DLDDKGCPAE

SEQ ID NO: 147 - protein, 10 aa, synthetic construct
  REGION 1..10: note = peptide with the consensus motif
DKGCPAEQRA

SEQ ID NO: 148 - protein, 10 aa, synthetic construct
  REGION 1..10: note = peptide with the consensus motif
CPAEQRASPL

SEQ ID NO: 149 - protein, 10 aa, synthetic construct
  REGION 1..10: note = peptide with the consensus motif
EQRASPLTGS

SEQ ID NO: 150 - protein, 10 aa, synthetic construct
  REGION 1..10: note = peptide with the consensus motif
ASPLTGSGSG

SEQ ID NO: 151 - protein, 15 aa, synthetic construct
  REGION 1..15: note = peptide with the consensus motif
EEGACQPCPINCTHS

SEQ ID NO: 152 - protein, 15 aa, synthetic construct
  REGION 1..15: note = peptide with the consensus motif
ACQPCPINCTHSCVD

SEQ ID NO: 153 - protein, 15 aa, synthetic construct
  REGION 1..15: note = peptide with the consensus motif
PCPINCTHSCVDLDD

SEQ ID NO: 154 - protein, 15 aa, synthetic construct
  REGION 1..15: note = peptide with the consensus motif
INCTHSCVDLDDKGC

SEQ ID NO: 155 - protein, 15 aa, synthetic construct
  REGION 1..15: note = peptide with the consensus motif
THSCVDLDDKGCPAE

SEQ ID NO: 156 - protein, 15 aa, synthetic construct
  REGION 1..15: note = peptide with the consensus motif
CVDLDDKGCPAEQRA

SEQ ID NO: 157 - protein, 15 aa, synthetic construct
  REGION 1..15: note = peptide with the consensus motif
LDDKGCPAEQRASPL

SEQ ID NO: 158 - protein, 15 aa, synthetic construct
  REGION 1..15: note = peptide with the consensus motif
KGCPAEQRASPLTGS

SEQ ID NO: 159 - protein, 15 aa, synthetic construct
  REGION 1..15: note = peptide with the consensus motif
PAEQRASPLTGSGSG

SEQ ID NO: 160 - protein, 10 aa, synthetic construct
  REGION 1..10: note = (G4S)2 linker
GGGGSGGGGS